(19) United States
(12) Patent Application Publication
(10) Pub. No.: US 2023/0149390 A1
(43) Pub. Date: May 18, 2023

(54) COMPOSITIONS FOR OPIATE AND OPIOID PREVENTION AND REVERSAL, AND METHODS OF THEIR USE

(71) Applicant: Torralva Medical Therapeutics LLC, Portland, OR (US)

(72) Inventor: Phillip R. Torralva, Portland, OR (US)

(73) Assignee: Torralva Medical Therapeutics LLC, Portland, OR (US)

(21) Appl. No.: 18/154,752

(22) Filed: Jan. 13, 2023

Related U.S. Application Data
(63) Continuation of application No. 17/266,960, filed on Feb. 8, 2021.
(60) Provisional application No. 62/716,291, filed on Aug. 8, 2018.
(60) Provisional application No. 62/828,914, filed on Apr. 3, 2019.

(51) Int. Cl. A61K 31/485; A61P 25/36; A61K 31/137; A61K 31/18; A61K 31/225; A61K 31/40; A61K 31/4166; A61K 31/4178; A61K 31/454; A61K 31/46; A61K 31/517; A61K 31/5377; A61K 31/5517

(52) U.S. Cl. CPC A61K 31/485; A61P 25/36; A61K 31/137; A61K 31/18; A61K 31/225; A61K 31/40; A61K 31/4166; A61K 31/4178; A61K 31/454; A61K 31/46; A61K 31/517; A61K 31/5377; A61K 31/5517

(57) ABSTRACT

Compositions are provided including a Mu opioid receptor antagonist, and an α2-adrenergic receptor agonist; or an α1 adrenergic receptor antagonist, together with one or more of a mu (or opioid receptor subtype) antagonist or agonist, (2) a vasopressor, (3) an anticholinergic agent and/or cholinergic agents, (4) a combined alpha-1 adrenergic antagonist and anticholinergic, (5) a paralytic or muscle relaxant, (6) a respiratory accelerant, (7) a GABA complex antagonist, (8) an anti-seizure/membrane stabilizer agent, (9) an α1 adrenergic receptor agonist, and/or (10) an α2 adrenergic receptor agonist; and a pharmaceutically acceptable carrier. Also provided are methods of preventing or reversing effects in a subject (including muscle and chest wall rigidity, laryngospasm, WCS, and/or respiratory depression) arising from intentional or accidental opioid or opiate exposure.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE DISCLOSURE

This disclosure relates to compositions and methods to treat (e.g., reverse and/or prevent) opiate and opioid effects in a subject. It further relates to preventing or reversing opioid/opiate overdose.

### BACKGROUND OF THE DISCLOSURE

In October of 2017, the opioid abuse epidemic was declared a national “public health emergency” in the United States of America. This declaration was based on the findings of a study by the Opioid and Drug Abuse Commission (ODAC), including that opioid-related deaths had risen from 4,000 in 1999 to over 64,000 in 2016. Opioid overdose the leading cause of death for Americans under the age of 50 (Rudd et al., Morb Mortal Wkly Rep 65:1445-1452, 2016). The Commission's report included a finding that the opioid epidemic had cost the U.S. an estimated $504 billion in 2015 alone, and is expected to cost over $1 trillion for 2018-2020.

The Center for Disease Control (CDC) reported that the highly potent synthetic opioid Fentanyl (Sublimaze™) and its analogues were the cause of death in >50% of U.S. deaths related to opioids in 2016 and estimated to be >70% for 2017 and 2018 (O'Donnell & Halpin, Synthetic Fentanyl deaths rise in American opioid epidemic. U.S. CDC, Oct. 27, 2017).

In 2018, the World Health Organization predicted a rapid global rise of these numbers as fentanyl and its analogues are increasingly available and as it becomes weaponized (e.g. aerosolization, environmental/water contamination) more frequently by various military, government and criminal organizations. The risk of significant harm to large urban populations is of significant concern due to the simple fact that fentanyl and its more potent analogues are several orders of magnitude more potent and rapid in causing death than either sarin gas or anthrax. and there are no effective treatments universally available to address its unique side effect profile (Riches et al., J Anal Toxicol. 36(9):647-656, 2012; Chemical weapon for sale: China's unregulated narcotic: FENTANYL. Oct. 7, 2016 Seattle Times).

Similarly the U.S. Center for Disease Control (CDC) has identified fentanyl and F/FAs as America's deadliest drug and fentanyl is currently being reviewed for classification as a “weapon of mass destruction” (WMD) by the U.S. Dept. of Homeland Security. Thus, the development of more effective treatments for overdose from accidental or weaponized exposure to fentanyl and fentanyl analogues (F/FA) by civilian populations is critical. F/FAs, in fact, have already been weaponized. In the “Moscow Theater incident” of 2002, mass civilian casualties occurred when carfentanil was used to manage a hostage crisis and caused mass civilian casualties. In spite of the administration of the mu opioid receptor (MOR) antagonist, naloxone, to treat the toxic exposure of these civilians to carfentanil, acute symptoms continued to develop, resulting in many fatalities (Riches et al., J Anal Toxicol 36(9):647-656, 2012; Pilch & Dolnik, The Moscow Theater Hostage Crisis: The Perpetrators, their Tactics, and the Russian Response. 2003. 8(3): p. 577) indicating that F/FA-induced rapid death is likely not mediated by MORs. Similarly, numerous public health and first responder reports indicate the failure of high dose naloxone to resuscitate overdose from illicit F/FA use, making F/FAs the number one cause of death in U.S. adults ages 18-50. With public health data increasingly indicating that naloxone is ineffective at decreasing F/FA-induced rapid fatality in the current U.S. opioid crisis, the development of reversal and prophylaxis drugs for chemical weapon defense is critical. (Current publicly funded research efforts for this development is of low priority and) Currently there are no Food and Drug Administration (FDA)-approved pharmacotherapies specifically for rescue treatment of F/FA-induced WCS or that can treat the unique side effect profile of F/FAs.

Like all narcotic opiates when given in sufficient quantity, fentanyl can induce significant, dose-dependent respiratory depression (RD) and apnea. Left untreated or treated inadequately, opioid-induced respiratory depression leads to hypoxia and death.

F/FAs are unique in that they can also rapidly induce severe muscle rigidity in the chest wall, diaphragm (Fentanyl or F/FA induced respiratory muscle rigidity—FIRMR), and spasm of the larynx (laryngospasm) resulting in vocal cord closure well within the therapeutic ranges used for analgesia (Grell et al., Anesth Analg 49(4):523-532, 1970; Streisand et al., Anesthesiology 78(4):629-634, 1993; Bennet et al., Anesthesiology 8(5):1070-1074, 1997; Coruh et al., Chest. 143(4):1145-1146, 2013; Ackerman et al., Anesth Prog 37(1):46-48, 1990; McClain et al., Clin Pharmacol Ther. 28:106-114, 1980). This combination of (FIRMR) and laryngospasm are also clinically known as “wooden chest syndrome” (WCS), usually occurs within 1-2 minutes after rapid injection and lasts ˜8-15 minutes. Rapidity of injection is the key determinant of the severity and duration of the FIMR (Grell et al., supra). The resulting rigidity reduces chest wall compliance and makes rescue-assisted ventilation extremely difficult outside of a critical care setting or operating room. Intervention for WCS must be immediate and aggressive to avoid death and usually includes treatment with a muscle paralytic and endotracheal intubation to secure the airway. This has been the method of choice since the underlying mechanism in humans remains unknown outside of this disclosure.

The need to combat opiate and opioid overdose is urgent, immediate, and rapidly increasing. There are currently no molecules or compounds that have been designed or exist for this specific purpose.

### SUMMARY OF THE DISCLOSURE

This disclosure examines and describes mechanisms developed through the inventor's clinical observation and experience with WCS in the fields of anesthesiology and addiction medicine, and the inventor's clinical observations demonstrating that WCS is the key cause of rapid death and escalating numbers of death in the current F/FA driven opioid crisis.

Provided herein is a description of the systematic development of a new generation of opioid reversal drugs and treatments designed to simultaneously and effectively antagonize both mu opioid receptor and other opioid receptor subtypes (kappa and delta) and the receptor(s) involved with fentanyl induced muscle rigidity (FIMR), fentanyl induced respiratory muscle rigidity (FIRMR), vocal cord closure (laryngospasm), and/or wooden chest syndrome (WCS=FIRMR+laryngospasm). It has surprisingly been discovered (based on animal models) that in addition to mu opioid receptors, F/FAs binding of alpha adrenergic and cholinergic receptors (e.g. muscarinic and nicotinic) contributes to and may be the most significant underlying cause of WCS. Based in part on these discoveries, this disclosure provides a clear methodology for the development of effective treatment compounds for prophylaxis and reversal of overdose and toxicity from F/FAs.

Conventional opiate reversal technology (e.g. naloxone, naltrexone) exclusively targets the mu-opioid receptor, and to a lesser extent the opioid receptor subtypes (kappa and delta), and uses these mu-opioid receptor antagonists for pharmacologic reversal of opioid-induced respiratory depression and over-sedation from both morphine alkaloid derived and synthetic opioids (e.g. F/FAs, meperidine, methadone). As described herein, respiratory depression can occur with all opioids, but WCS syndrome appears to be a unique and lethal side effect of F/FAs that is clinically and neuropharmacologically distinct from morphine derived alkaloids and the effects of opioids at opioid receptors. It becomes more readily understood by the teachings herein that, due to the unique side effect profile of fentanyl and fentanyl analogs (F/FAs), conventional single therapy with naloxone is no longer adequate, safe or cost effective for treatment of overdose or toxic exposure related to F/FAs or “fentanyl-tainted heroin”.

The use of either pure fentanyl, fentanyl analogs (e.g. synthetic opioids) or the concurrent use of F/FAs with heroin or other morphine derived opiates (e.g. natural alkaloids), creates a unique problem in the conventional treatment of narcotic overdose (e.g. opiates and opioids). Solutions to this problem are by embodiments of the current disclosure. As demonstrated herein, F/FAs are mechanistically unique from morphine, particularly in their effects on the upper airway (larynx and vocal cords) and in WCS. This disclosure describes receptor populations that drive the clinical effects of WCS. These receptor populations in turn suggest a multi-site effect that requires multiple drugs in combination as a compound for optimal treatment (e.g., combinations of drugs that specific target mu opioid receptors, alpha-1 adrenergic receptors, and muscarinic cholinergic receptors). This disclosure teaches how to make these combination compounds and how to administer them for treatment and prevention (e.g. the conditions of administration).

Prior to the teachings herein, there was no focus on developing therapeutic agents specifically for the reversal or prophylaxis treatment of F/FA induced WCS This is due at least in part to a pervasive misunderstanding in the medical and research community of the basic mechanism of action (MOA) involved in F/FAs overdose, and a consistent misperception among health care professionals and researchers in addictionology that F/FAs are simply more potent versions of morphine and heroin. The misconception is that mu opioid receptor-mediated respiratory depression is the main cause of death after F/FA overdose, and therefore the administration of conventional medications (mu opioid receptor antagonists, such as naloxone) in larger amounts would seem to be the logical solution or treatment to compensate for the increased potency of F/FAs (Baumann et al., Trends Pharmacol Sci. 39(12):995-998, 2018). Similarly, the risk of negative complications due to high dose naloxone (e.g. pulmonary edema, cardiac arrhythmias) are not commonly known among these same practitioners. These two factors have contributed significantly to the ongoing morbidity and mortality from F/FAs related overdose, have limited the development of new drugs or compounds specifically for this purpose and the understanding of underlying mechanisms of F/FAs in WCS. The advances described herein address these problems directly.

This disclosure describes a re-conceptualization of the methodology for treating opioid overdose and F/FAs related overdose by using a “multi-systems treatment approach” through the use of compounds/combinations of molecules that concurrently target multiple physiologic systems and symptoms to optimize opioid overdose reversal involving F/FAs and combinations of F/FAs with heroin and other morphine derived alkaloids.

There is provided herein a platform of compounds that are all part of a single invention and singular outcome (overdose survival) that is adapted to variations in human physiology and adaptable to variations of opioid molecules overlapping in their mechanisms of overdose and death. These compounds share the same underlying mechanism and function of concurrently blocking or reversing the effects of natural opiate alkaloids, and/or the effects of synthetic opiate receptor agonists on opiate receptors and other receptor types, in the body and brain of mammalian system that contribute to the lethal effects of opiate and opioid overdose. For the purposes of this disclosure, opioid overdose with F/FAs includes WCS in addition to respiratory depression, and optimal treatment involves the concurrent treatment of both clinical presentations and their underlying mechanisms. The technology described here provides a series of compounds and composition using established recognized therapeutic compounds (drugs) and other molecules that selectively bind receptors and receptor subtypes in brain and body regions responsible for FIMR and F/FAs overdose-related physical sequelae (such as WCS/laryngospasm/FIRMR).

In specific embodiments, this disclosure offers a multimodal approach to concurrently affect central and peripheral effect sites of opiates and opioids, and favorably impact the physical symptoms of overdose such as vascular compromise; lowered hemodynamics, blood pressure, heart rate; increased vagal tone; chemoreceptor depression (carotid and aortic bodies); mu, delta, kappa opiate receptors agonism; α adrenergic receptors agonism/antagonism; and skeletal muscle-acetylcholine-(Ach) receptor activation; as may be needed to optimize rapidity and effectiveness of opioid reversal and to reduce mortality from F/FA related overdose, or as needed for prophylaxis against exposure. Specifically, the treatment for F/FA overdose and toxic exposure involves prevention of and/or reversal of laryngospasm and upper airway effects and chest wall and diaphragm rigidity that appear to be unique to F/FAs as mentioned previously.

Another aspect provided herein deals with overdose due to opiates/opioids and benzodiazepine sedative-hypnotic agents. In examples of this embodiment, a formulation combines a GABA receptor complex antagonist with one or more agents that antagonize respiratory depression and FIMR (e.g. flumazenil).

The current opioid crisis significantly increases the risk of direct toxin exposure to “first responders”, as well as the general public, by accidental, un-intentional, or intentional (e.g. malicious, terrorist activity, weaponization) environmental contamination. Prior to this disclosure, no prophylaxis agents existed outside of conventional treatment with a mu or opioid receptor antagonist. This disclosure addresses this problem by using similar conceptual technology as the provided immediate reversal agents, but utilizing a different mode of timing and duration to create long-acting or extended-release prophylaxis agents that address the F/FAs side effect profile.

Another aspect of the disclosure is in the provision of compounds or compositions that not only provide immediate reversal agents for F/FAs related overdose, but also provide prophylaxis formulations as part of the development “platform”, that are designed specifically to provide prophylactic receptor antagonism to minimize or prevent the effects of FIRMR/WCS from F/FAs overdose or that may occur from environmental exposure. Prophylaxis agents are ideal for “first responders” or individuals who are not habitual opiate or illicit opiate users that may be at risk for environmental exposure to F/FAs. Examples of such formulations include a minimum composition of (1) an extended release Mu opioid receptor or opioid receptor (mu, kappa, delta receptor subtypes) antagonist, (2) combined with an α adrenergic antagonist/agonist, and (3) either an anticholinergic agent such as atropine or an M3 muscarinic receptor agonist (such as pilocarpine) that can override the effects of fentanyl at muscarinic receptors (either by generalized antagonism of all M1-M5 receptors or targeted agonism at M3, which is unique to F and F/FAs), (4) and a cholinergic agent (muscarinic receptor antagonist/anticholinergic, M3 receptor agonist or a nicotinic receptor general or selective agonist).

The needs of first responders are different from those of individuals who are illicit and/or habitual opiate users, because the latter group would be unlikely to allow, consent to, or utilize prophylaxis treatment containing a Mu opioid receptor antagonist or opioid receptor (mu, kappa, and/or delta receptor subtypes) antagonist, as these would prevent them from feeling/]experiencing the euphoric effects of opioids/opiates and would likely precipitate significant and prolonged opiate withdrawal symptoms. In this particular case, a habitual opiate user may be willing to use a prophylaxis agent that protects against WCS/FIRMR from F/FAs such as one that simply contains a combination of α-adrenergic antagonists/agonists in a compound with a cholinergic agent (muscarinic receptor antagonist/anticholinergic, M3 receptor agonist or a nicotinic receptor general or selective agonist) designed to mitigate the side effect profile of the prophylaxis agents (e.g. α-adrenergic antagonists Tamsulosin and Prazosin in a 1:0.5 ratio with α-adrenergic agonist Phenylephrine—see Table 1 for representative dosing). This disclosure recognizes and addresses the necessity for formulations that specifically address the needs and characteristics of each treatment group or population.

Also provided are formulations that specifically address mitigation of the side effect profile of α-adrenergic antagonists, by using vasoactive agents (such as the α-adrenergic agonist phenylephrine) to stabilize blood pressure in the face of the significant hypotension that may occur with moderate to high dose α adrenergic antagonists or an anticholinergic agent (e.g. atropine) given for the dual effect of preventing bradycardia and to modify possible fentanyl M3 antagonist effects on vagal motor nuclei controlling laryngeal muscle patency. Thus, there are provided herein formulations that minimize or mitigate the side effect profiles of α-adrenergic antagonists either by creating synergy to reduce side effect profile, or through directly designed formulations that minimize or mitigate side effect profiles of the α adrenergic antagonist agents.

Also provided herein are formulations which specifically address the needs and characteristics of different physical and clinical presentation. Described herein is a detailed conceptual framework that involves using multiple agents with complementary supportive or opposing effects to modify side effect profiles of F/FAs and to optimize immediate treatment (reversal). The different compounds can be combined with a vital sign guideline or clinical presentation chart, offering specific hemodynamic parameters to determine the compound to be used, in other words, formulation selection specific to hemodynamic profile (e.g. ephedrine and phenylephrine can be used either singly or in combination for low blood pressure Systolic<90 mmHg or Diastolic<50 mmHg, atropine and glycopyrrolate can be used for bradycardia with HR<50 BPM).

This disclosure recognizes and addresses the necessity for formulation development that specifically addresses the needs, skill sets, and medical training level of different untrained users and a range of medical practitioners.

The immediate reversal formula for F/FA overdose or toxic exposure is formulated, in various embodiments, as either a non-prescription, minimal training-required version or a more sophisticated, prescription-only version for a provider who is medically trained in airway (AW) and hemodynamics management. Examples of the minimal or untrained user formulations contain a mu opioid receptor antagonist or another opioid receptor (mu, kappa, and/or delta receptor subtypes) antagonist, an anticholinergic agent (muscarinic antagonist e.g. atropine) or muscarinic agonist (e.g. pilocarpine M3) and an α-1A adrenergic antagonist, and optionally a vasoactive agent (such as phenylephrine) to maintain blood pressure. Examples of the formulations for a provider medically trained in AW and hemodynamics management may contain a combination of a mu, kappa and/or delta receptor antagonist, an α-1A or D adrenergic antagonist, a vasoactive agent (such as phenylephrine) to maintain blood pressure, an anti-cholinergic or M3 agonist to prevent bradycardia and/or upper airway effects, and a rapid acting muscle relaxant/paralytic (such as succinylcholine or rocuronium) in a dose range that relaxes skeletal muscle without causing airway (AW) compromise or to fully secure the AW. Optionally, if conventional naloxone fails and one of the multi-drug formulations in this disclosure fails to reverse WCS, a full dose of muscle paralytic can be administered along with endotracheal intubation to secure the airway; these actions are taken by personnel medically trained to do so. For instance, if the analogue is so potent that its effect is not overcome by the compounds listed here, a failsafe is to secure the airway with a full dose of a muscle paralytic, intubate the patient, and ventilate with 100% oxygen. It is anticipated that most of the current F/FAs will be treated with the compounds listed here, some of which are combined with muscle paralytics.

Provided herein are myriad compositions and methods for treating multiple levels of mechanism of action (MOA) of opiate receptor activation in different organ systems of the body, such as the vascular system, heart, different brain regions, receptor cells in aorta and carotids and pontine and medullary motor nuclei controlling the AW and respiratory muscles of the chest wall and abdomen.

Additional details regarding various embodiments are provided further below.

### BRIEF DESCRIPTION OF THE DRAWING

FIG. 1 Proposed Mode of Action (MOA) of Fentanyl at Locus Coeruleus and Medulla: Among humans, the clinical model of WCS demonstrates the significant involvement of the larynx and vocal cords and laryngospasm is the key feature of WCS. In summary, WCS is a syndrome that includes laryngospasm, respiratory muscle rigidity/contraction, cardiovascular compromise and a concurrent decline in hepatic metabolism. Each component can be explained as a consequence of alpha-1 adrenergic, noradrenergic and cholinergic receptor activity in addition to mu opioid receptor activation. The path begins with increased norepinephrine in the LC by fentanyl (indirectly), followed by activation and/or inhibition of noradrenergic and cholinergic signal paths through the CNS and terminates on laryngeal muscles resulting in laryngospasm. The mechanism has several pathways: A.1). F/FA activation of the LC by systemic administration in peripheral vein or lungs. F/FAs enter the CNS via the carotid and vertebral arteries, cross the blood brain barrier and bind mu opioid receptors on GABA interneurons in the LC, which inhibits GABA cell firing, prevents GABA release and results in increased release of norepinephrine from presynaptic terminals of the LC. As norepinephrine levels increase in the synapse from these combined mechanisms, norepinephrine binds alpha-1 adrenergic receptors on the post synaptic terminal and increases the noradrenergic outflow of the LC to several sites that ultimately impact muscle tone in the chest wall, abdomen, diaphragm and laryngeal muscles. Noradrenergic signals from the LC can increase muscle contractility via coerulospinal sympathetic fibers to spinal motor neurons, superior cervical and other sympathetic ganglia (T1-L2) that terminate on skeletal muscle (e.g. chest wall, diaphragm, laryngeal muscles), or via noradrenergic fibers to respiratory motor centers in the medulla (Dorsal respiratory group—DRG and Ventral respiratory group—VRG) and motor efferents in the nucleus ambiguus vagal nuclei controlling innervation of the intrinsic laryngeal muscles. A.2) Vagus nerve: The vagus nerve originating in the nucleus ambiguus of the medulla exits the brain to supply the intrinsic striated muscles of the larynx, providing bilateral motor innervation via the external branch of the superior laryngeal and the recurrent laryngeal nerves. Cricothyroid muscle is the sole tensor of vocal cords innervated via the superior laryngeal nerve. All other larynx muscles are innervated by the recurrent laryngeal nerve (e.g. lateral cricoarytenoid (LCA), posterior cricoarytenoid (PCA) and the thyroarytenoid (TA) muscles. Importantly, the PCA muscles are the sole abductors of the vocal cords and the LCA and TA muscles adduct the vocal cords. Dominant vagal parasympathetic tone to the intrinsic muscles of the larynx allows the abduction via the PCA and keeps glottis/airway open for relaxed inspiration and expiration. A.3) Sympathetic fibers in the vagus nerve originating from the LC contribute innervation directly and indirectly to the intrinsic muscles of the larynx through several pathways coerulospinal fibers terminate in spinal motor neurons that then relay noradrenergic efferent signals from the CNS to sympathetic ganglia and superior cervical sympathetic ganglion (SCG) that supplies the head and neck with sympathetic innervation. Specifically, the SCG provides sympathetic fibers to the terminal branches of the vagus nerve that serve as the sole innervation of the intrinsic muscles of the larynx. Increased sympathetic outflow from the LC and offers a plausible mechanism whereby norepinephrine can rapidly activate vocal cord adductors to cause severe laryngospasm, particularly in a system where cholinergic/parasympathetic tone is diminished or compromised (e.g. anticholinergic drug effects). A.4) Cholinergic innervation of the VC/larynx: The LC controls autonomic nuclei in the medulla, specifically the nucleus ambiguus, which gives rise to the efferent motor fibers of the vagus nerve. Alternatively, fentanyl can directly bind cholinergic nuclei in the nucleus ambiguus. acting as a selective M3 antagonist and could facilitate selective isolation of M1 and M2 receptors for ACh, binding, resulting in increased relaxation of laryngeal abductor muscles and diminished opposition to sympathetically mediated laryngeal adductor contraction. A.5) Diaphragm, Abdomen and Thoracic wall innervation: the main muscles of respiration include the diaphragm, intercostals and abdominal wall muscles. Diaphragm receives its motor nerve impulses from the medullary centers via the phrenic nerve and sympathetic nerve fibers from the cervical sympathetic chain. The intercostal muscles are stimulated by way of cervical, thoracic and lumbar motor nerves and spinal motor neurons that terminate on skeletal muscles of the thorax and abdomen. A.6) Medulla DRG VRG: Respiration increases or decreases via afferent signals from the vagus and glossopharyngeal nerves via peripheral chemo and mechanical receptors and mechanical changes in the lung and airway. These signals return to respiratory centers in the pons, medulla (i.e., dorsal respiratory group [DRG]) and ventral respiratory group (VRG). DRG controls initiation of inspiration via motor nerves to diaphragm and external intercostal muscles. VRG contains inspiratory and expiratory neurons and controls laryngeal/pharyngeal muscles, diaphragm, abdominal and intercostals. Additionally, vagus nerve fibers contain mu opioid receptors that innervate stretch receptors in lung and can send afferent signals to vagal nuclei when activated by opioid binding causing a cessation of inspiratory drive to prevent over-inflation of the lung via inhibitory signals to the DRG to cease further inspiration, and may include VRG-mediated closure of the vocal cords. A.7) Cardiac Function in WCS: Cardiac output can be inhibited by vagus nerve activation in the nucleus ambiguus. Fentanyl binds mu opioid receptors in vagal nuclei and GABA interneurons of the nucleus ambiguus, and can cause severe bradycardia and decreased cardiac output, with direct consequences for cerebral and hepatic perfusion pressures. As noted herein, fentanyl also antagonizes alpha 1 adrenergic receptor subtypes in a rank order of 1B>1A>>1D and may have significant consequences on cardiac function via selective distributions of these receptors in coronary arteries. These proposed cardiac mechanisms may explain the rapid onset of vascular, hepatic and CNS effects described in public health/autopsy data including: 1) rapid onset of cyanosis, 2) immediate loss of consciousness (central thalamocortical inhibition and/or decreased cerebral perfusion decrease) and 3) decreased hepatic metabolism. A.8) Hepatic Function in WCS: By decreasing cardiac output, high dose fentanyl can secondarily cause decreased hepatic artery perfusion and/or potentially decreased cerebral perfusion, resulting in a significant malfunction in fentanyl metabolism and a rapid loss of consciousness.

### DETAILED DESCRIPTION

The present disclosure takes advantage of combined and, in some cases, synergistic effect(s) between mu and/or opioid receptor antagonists, cholinergic agents and one or more of α-adrenergic agonists/antagonists, anticholinergics, respiratory accelerants, vasoactive agents and muscle relaxants/paralytics, to provide novel combinations having utility in the reversal of or prophylaxis against opioid/opiate effects (e.g. F/FAs and morphine derived alkaloids). These compounds are designed to treat respiratory depression from conventional morphine derived opiates and/or fentanyl and fentanyl analogue (F/FA) induced respiratory muscle rigidity (FIRMR), wooden chest syndrome (WCS). Different and specific formulations described here can be used as reversal drugs, prophylaxis against F/FA environmental exposure, and for polysubstance exposure reversal (e.g. F/FAs and/or morphine derivatives combined with benzodiazepines. Embodiments of the described methods involve identification of treatment individuals or groups, treatment by clinical presentation of individual subjects (for instance mammalian subjects, such as humans), and provision of treatment formulation(s) as per the expected or known skill set of the user. Overall, these are largely reiterations of how to use the focus of the herein described technology, which is the compositions and compounds described.

Formulations designed for “multi-systems treatment approach”: This disclosure describes a re-conceptualization of the methodology for treating opioid overdose and F/FA related overdose by using a “multi-systems treatment approach” through the use of compounds/combinations of molecules that concurrently target the multiple physiologic systems affected by F/FAs and the symptoms of these effects, to optimize opioid overdose reversal and/or provide prophylaxis against these effects.

In one embodiment, there is provided a platform of compounds that concurrently block or reverse the effects of natural opiate alkaloids, and/or the effects of synthetic opiate receptor agonists on opiate receptors and other receptor types, in the body and brain of mammalian systems that contribute to the lethal effects of opiate and opioid overdose. The technology described here provides a series of compounds and composition using established and recognized therapeutic compounds (drugs) and other molecules that selectively bind receptors and receptor subtypes (alpha-1 adrenergic and cholinergic receptors) in brain and body regions responsible for WCS/FIRMR and other F/FAs overdose-related physical sequelae.

Formulation that is Multimodal: In specific embodiments, this disclosure offers a multimodal approach to concurrently affect central and peripheral effect sites of opiates and opioids, and favorably impact the physical symptoms of overdose such as vascular compromise; lowered hemodynamics, blood pressure, heart rate; increased or decreased vagal tone; chemoreceptor depression (carotid and aortic bodies); mu, delta, kappa opiate receptors agonism; α-adrenergic receptors agonism/antagonism; and skeletal muscle-acetylcholine (Ach) receptor activation; as may be needed to optimize rapidity and effectiveness of opioid reversal and to reduce mortality from F/FAs related overdose, or as needed for prophylaxis against exposure.

Formulation for Broad-spectrum opiate reversal: Utilizing the system and compositions described herein does not require one to distinguish the type of opiate or opioid ingestion prior to treatment. It therefore can be used in all manner of opioid overdose situations, and offers the unique ability to treat overdoses due to either single opiates or opioid mixtures that, for instance, involve morphine derivatives and synthetic opioids (both piperidine and non-piperidine derived).

Formulation for Polysubstance: Another aspect provided herein deals with overdose due to opiates/opioids and benzodiazepine sedative-hypnotic agents. In examples of this embodiment, a formulation combines a GABA receptor complex antagonist with one or more agents (e.g. respiratory accelerants) that antagonize respiratory depression and FIRMR (e.g. flumazenil and doxapram).

Formulation for Prophylaxis: In the current opioid crisis, the sheer potency of the F/FAs being brought into urban and rural communities by the illicit drug trade—and by those who use these IV drugs illicitly—significantly increases the risk of direct toxin exposure to “first responders” (e.g. emergency medical technicians (EMTs), paramedics, firefighters, law enforcement personnel, emergency room medical providers, and military personnel), as well as the general public, by accidental, un-intentional, or intentional (e.g. malicious or terrorist activities etc.) environmental contamination. There have been a number of well-documented cases of opioid overdose from first responder F/FAs environmental exposures requiring treatment/hospitalization as well as civilian deaths from weaponization in pubic settings.

Prior to this disclosure, there are no prophylaxis or reversal agents for F/FAs other than conventional treatment with a mu receptor antagonist. This disclosure addresses this problem by using similar conceptual technology as the immediate reversal agents, but utilizes a different mode of timing and duration to create long-acting or extended-release prophylaxis agents that address the F/FAs side effect profile.

Formulations designed specific to “first responders”: In addition to immediate reversal agents for F/FAs related overdose, this disclosure provides prophylaxis formulations as part of the development “platform”, that are designed specifically to provide prophylactic receptor antagonism that minimizes or prevents the effects of FIRMR and/or WCS from F/FA overdose that may occur from environmental exposure. The prophylaxis agents are ideal for “first responders” or individuals who are not habitual opiate or illicit opiate users, that may be at risk for environmental exposure to F/FAs. Such formulations include a minimum composition of an extended release Mu and/or opioid receptor subtype antagonist combined with an α adrenergic antagonist/agonist and a cholinergic agent (muscarinic receptor antagonist/anticholinergic, M3 receptor agonist and possibly a nicotinic receptor general or selective agonist).

“First responders” are distinguishable from individuals who are illicit and/or habitual opiate users, because the latter group would be unlikely to consent to, or utilize prophylaxis treatment containing opioid antagonists, as these would prevent them from feeling/experiencing the euphoric effects of opioids/opiates and would likely precipitate significant and prolonged opiate withdrawal symptoms. This disclosure recognizes and addresses the necessity for formulations that specifically address the needs and characteristics of each treatment group or population.

Formulations for side-effect mitigation: Also provided are formulations that specifically addresses the mitigation of the side effect profile of α adrenergic antagonists, by using vasoactive agent(s) (such as phenylephrine) to stabilize blood pressure in the face of the significant hypotension that may occur with moderate to high dose α adrenergic antagonists. α-adrenergic antagonists are well known to cause severe hypotension particularly with the first dose or exposure and is well described in the medical literature as the “first dose effect or phenomenon” which can be associated with severe hypotension first exposure to an α-adrenergic antagonist. High doses of α adrenergic agents were used in animal experiments and shown to be effective for inhibiting the occurrence of FIMR. However, these doses are 2-5× higher (mg/kg) than doses used in human subjects to treat various medical conditions such as hypertension or benign prostatic hypertrophy, and in most instances may cause life-threatening hypotension. Thus, there are provided herein formulations that minimize or mitigate the side effect profiles of α-adrenergic antagonists either by creating synergy to reduce side effect profile, or through directly designed formulations that minimize or mitigate side effect profiles of the α adrenergic antagonist agents.

Formulations design and selection specific to hemodynamic profile: Also provided herein are formulations which specifically address the needs and characteristics of different physical and clinical presentation. Described herein is a clear, detailed conceptual framework that involves using multiple agents with complementary supportive or opposing effects to modify side effect profiles to optimize immediate treatment. The different compounds can be combined with a vital sign guideline chart, offering specific hemodynamic parameters to determine the compound to be used in other words, formulation selection specific to hemodynamic profile.

For instance, in representative embodiments (see also Table 1, provided in two parts), in case of a suspected F/FA, or F/FA with morphine derived alkaloid, overdose and the following clinical presentation:
1) suspected opioid overdose (OD), unresponsive patient with rapid, “thready” pulse indicating low blood pressure (BP): treat with a formulation or dosing regimen including Naloxone, Prazosin or Alpha-1A and/or 1D selective antagonist and Phenylephrine and/or a cholinergic agent (e.g. Pilocarpine—M3 agonist). (Rapid pulse would make an anticholinergic contraindicated because it would make pulse even faster or precipitate an arrhythmia.)
2) suspected opioid OD with slow, “thready” pulse and low BP: treat with a formulation or dosing regimen including formulation with Naloxone, Prazosin or Alpha-1A and/or 1D selective antagonist, Ephedrine and glycopyrrolate and/or atropine. (Slow pulse would make an anticholinergic a preferred agent because it would make pulse faster.)
3) suspected opioid OD with prominent rigidity: treat with a formulation or dosing regimen including formulation with Naloxone, Prazosin or Alpha-1A and/or 1D selective antagonist and a cholinergic agent (muscarinic receptor antagonist/anticholinergic, M3 receptor agonist) and glycopyrrolate and/or Succinylcholine or Rocuronium (though only if responder is airway trained, airway equipment is available, and patient is unresponsive to treatment of core compound).
4) suspected opioid OD is pulseless and low pressure: treat with a formulation or dosing regimen including formulation with Naloxone, Alpha-1A and/or 1D selective antagonist (e.g. tamsulosin, terazosin), ephedrine, glycopyrrolate/atropine, and/or epinephrine.
5) uncertain presentation: In the event that the medical provider is uncertain of physiologic or clinical presentation in “Suspected Opioid Overdose”, use the baseline “Non-Medical” dosing kit until vital signs are apparent and directional, then follow algorithm as above in clinical scenarios 1-4.

Formulations designed and selection specific to user experience/training level: This disclosure recognizes and addresses the necessity for formulation development that specifically addresses the needs, skill sets, and medical training level of different untrained users and a range of medical practitioners.

The immediate reversal formula is formulated, in various embodiments, as either a non-prescription, minimal training-required version or a version for a provider who is medically trained in airway (AW) and hemodynamics management. Examples of the minimal or untrained user formulations contain a mu or other opioid receptor (mu, kappa, delta and/or receptor subtypes) antagonist and an α-1D adrenergic antagonist, an α-1D adrenergic antagonist in a composition ratio with a non-selective α1-adrenergic antagonist (e.g., 1:1, 2:1, 3:1 ratio in favor of the selective α-1D agent) and optionally a vasoactive agent (such as phenylephrine) to maintain blood pressure and/or a cholinergic agent (muscarinic receptor antagonist/anticholinergic, M3 receptor agonist. Examples of the formulations for a provider medically trained in AW and hemodynamics management contain a mu or opioid receptor (mu, kappa, delta receptor subtypes) antagonist, an α-1A adrenergic antagonist, a vasoactive agent (such as phenylephrine) to maintain blood pressure, an anti-cholinergic to prevent bradycardia and antagonize laryngeal effects and/or a cholinergic agent (muscarinic receptor antagonist/anticholinergic, M3 receptor agonist, and a rapid acting muscle relaxant/paralytic (such as succinylcholine or rocuronium) in doses that can relax skeletal muscle without causing AW compromise.

TABLE 1
Base Dose Compounds (BDC) and Treatment (provided in four parts).
Table 1: BASE DOSE COMPOUNDS (BDC) and TREATMENT ALGORITHMS

| | | | | Optional | | | |
|---|---|---|---|---|---|---|---|
| | | | | | | | RESPI- |
| | | | | VASO- | ANTICHO- | | RATORY |
| | | | ALPHA-1 | PRESSOR | LINERGICS | PARA- | ACCELERANTS |
| DRUG CLASSES * | | MU | S/NS | (VP) | (AC) | LYTICS | (RA) |
| MODE | IMMEDIATE REVERSAL | | | | | | |
| | FENTANYL & FENTANYL ANALOGUES | | | | | | |
| | NON- | Naloxone | NS- Prazosin | N/A | N/A | N/A | N/A |
| | MEDICAL | (1-4 mg) | (0.2-0.5 mg) | | | | |
| | | 1-4 doses | and/or | | | | |
| | | of BDC | S- Tamsulosin | | | | |
| | | | (0.2-0.4 mg) | | | | |
| | | | 1-4 doses | | | | |
| | | | of BDC | | | | |
| | MEDICAL W/O | Naloxone | NS- Prazosin | Phenylephrine | Atropine | N/A | Doxapram |
| | AIRWAY W/ | (1-4 mg) | (0.2-0.5 mg) | (50-200 mcg) | (0.5 mg) | | (10 mg) and/or |
| | Hemodynamic | 1-4 doses | and/or | 1-4 doses | and/or | | (0.5 mg/kg- |
| | Monitoring | of BDC | S- Tamsulosin | of BDC | Glycopyrrolate | | 1 mg/kg) |
| | Available | | (0.2-0.4 mg) | | (0.2 mg) | | 1-4 doses |
| | | | 1-4 doses | | 1-4 doses | | of BDC |
| | | | of BDC | | of BDC | | Nicotine |
| | MEDICAL W | Naloxone | NS- Prazosin | Phenylephrine | Atropine | Succinylcholine | Doxapram |
| | AIRWAY W/ | (1-4 mg) | (0.2-0.5 mg) | (50-200 mcg) | (0.5 mg) | (1-3 mg) | (10 mg) and/or |
| | Airway | 1-4 doses | and/or | 1-4 doses | and/or | 1-4 doses | (0.5 mg/kg- |
| | Equipment | of BDC | S- Tamsulosin | of BDC | Glycopyrrolate | of BDC | 1 mg/kg) |
| | Available | | (0.2-0.4 mg) | | (0.2 mg) | | 1-4 doses |
| | | | 1-4 doses | | 1-4 doses | | of BDC |
| | | | of BDC | | of BDC | | |
| | CLINICAL PRESENTATION † | | | | | | |
| | 1) Suspected | Naloxone | NS- Prazosin | Phenylephrine | N/A | +/− | +/− |
| | Opioid OD, | (1-4 mg) | (0.2-0.5 mg) | (50-200 mcg) | | | |
| | unresponsive | 1-4 doses | and/or | 1-4 doses | | | |
| | patient with | of BDC | S- Tamsulosin | of BDC | | | |
| | RAPID, | | (0.2-0.4 mg) | | | | |
| | “thready” | | 1-4 doses | | | | |
| | pulse | | of BDC | | | | |
| | indicating | | | | | | |
| | LOW BP | | | | | | |
| | 2) Suspected | Naloxone | NS- Prazosin | Ephedrine | Atropine | +/− | +/− |
| | Opioid OD | (1-4 mg) | (0.2-0.5 mg) | (5-10 mg) | (0.5 mg) | | |
| | with SLOW, | 1-4 doses | and/or | 1-4 doses | and/or | | |
| | “thready” | of BDC | S- Tamsulosin | of BDC | Glycopyrrlate | | |
| | pulse and | | (0.2-0.4 mg) | | (0.2 mg) | | |
| | LOW BP | | 1-4 doses | | 1-4 doses | | |
| | | | of BDC | | of BDC | | |
| | 3) Suspected | Naloxone | NS- Prazosin | N/A to avoid | Atropine | Succinylcholine | +/− |
| | Opioid OD | (1-4 mg) | (0.2-0.5 mg) | increased NA | (0.5 mg) | (1-3 mg) | |
| | with | 1-4 doses | and/or | tone in LC | and/or | 1-4 doses | |
| | prominent | of BDC | S- Tamsulosin | | Glycopyrrlate | of BDC | |
| | RIGIDITY or | | (0.2-0.4 mg) | | (0.2 mg) | | |
| | severe | | 1-4 doses | | 1-4 doses | | |
| | laryngospasm | | of BDC | | of BDC | | |
| | | | | | To maximize | | |
| | | | | | inhibition | | |
| | | | | | of vagal | | |
| | | | | | tone in the | | |
| | | | | | CNS/LC | | |
| | 4) Suspected | Naloxone | NS- Prazosin | Phenylephrine | Atropine | +/− | +/− |
| | Opioid OD is | (1-4 mg) | (0.2-0.5 mg) | (50-200 mcg) | (0.5 mg) | | |
| | pulseless-NO | 1-4 doses | and/or | 1-4 doses | and/or | | |
| | PULSE and | of BDC | S- Tamsulosin | of BDC | Glycopyrrlate | | |
| | LOW BP | | (0.2-0.4 mg) | Ephedrine | (0.2 mg) | | |
| | pressure | | 1-4 doses | (5-10 mg) | 1-4 doses | | |
| | | | of BDC | 1-4 doses | of BDC | | |
| | | | | of BDC | | | |
| | | | | Epinephrine**** | | | |
| | POLYSUBSTANCE | | | | | | |
| | NON- | Naloxone | NS- Prazosin | N/A | N/A | N/A | N/A |
| | MEDICAL | (1-6 mg) | (0.2-0.5 mg) | | | | |
| | | 1-6 doses | and/or | | | | |
| | | of BDC | S- Tamsulosin | | | | |
| | | | (0.2-0.4 mg) | | | | |
| | | | 1-4 doses | | | | |
| | | | of BDC | | | | |
| | MEDICAL W/O | Naloxone | NS- Prazosin | N/A due to | N/A due to | N/A | N/A due to |
| | AIRWAY W/ | (1-6 mg) | (0.2-0.5 mg) | seizure risk. | seizure risk. | | seizure risk. |
| | Hemodynamic | 1-6 doses | and/or | | | | |
| | Monitoring | of BDC | S- Tamsulosin | | | | |
| | avail. | | (0.2-0.4 mg) | | | | |
| | | | 1-4 doses | | | | |
| | | | of BDC | | | | |
| | MEDICAL W | Naloxone | NS- Prazosin | N/A due to | N/A due to | Succinylcholine | N/A due to |
| | AIRWAY | (1-6 mg) | (0.2-0.5 mg) | seizure risk. | seizure risk. | (1-3 mg) | seizure risk. |
| | W/ AW EQUIP | 1-6 doses | and/or | | | 1-4 doses | |
| | AVAIL. | of BDC | S- Tamsulosin | | | of BDC | |
| | | | (0.2-0.4 mg) | | | | |
| | | | 1-4 doses | | | | |
| | | | of BDC | | | | |
| | PROPHYLAXIS | | | | | | |
| | IV USER | | NS- Prazosin | Phenylephrine | Atropine | N/A | N/A |
| | | | (0.2-0.5 mg) | (25-200 | (0.5 mg) | | |
| | | | and/or | mcg/hour) in | and/or | | |
| | | | S- Tamsulosin | time release | Glycopyrrolate | | |
| | | | (0.2-0.4 mg) | PO tab OR IM | (0.1-0.2 | | |
| | | | | injection) | mg/hour in a | | |
| | | | | | time release | | |
| | | | | | PO tab) | | |
| | | | | | Scopolamine | | |
| | 1^{st} | Naltrexone | NS- Prazosin | Phenylephrine | N/A | N/A | N/A |
| | RESPONDER | (25-50 mg) | (0.2-0.5 mg) | (25-200 | | | |
| | | | and/or | mcg/hour, in | | | |
| | | | S- Tamsulosin | time release | | | |
| | | | (0.2-0.4 mg) | PO tab OR IM | | | |
| | | | | injection) | | | |

| | | Optional | | |
|---|---|---|---|---|
| | | GABA | COMBO | |
| | | COMPLEX | AGENTS | MUSCA- |
| | | ANTAG- | (alpha- | RINIC |
| DRUG CLASSES * | | ONISTS | 1 + AC) | AGENTS |
| MODE | IMMEDIATE REVERSAL | IMMEDIATE REVERSAL | | |
| | FENTANYL & FENTANYL ANALOGUES | FENTANYL & FENTANYL ANALOGUES | | |
| | NON- | N/A | | |
| | MEDICAL | | | |
| | MEDICAL W/O | N/A | Droperidol § | e.g. |
| | AIRWAY W/ | | (0.5 mg) | Pilocarpine♦ |
| | Hemodynamic | | 1-4 doses | (1-2.5 mg) |
| | Monitoring | | of BDC | 1-4 doses |
| | Available | | | of BDC |
| | | | | (SEE NOTE) |
| | MEDICAL W | N/A | Droperidol § | (SEE NOTE) |
| | AIRWAY W/ | | (0.5 mg) | |
| | Airway | | 1-4 doses | |
| | Equipment | | of BDC | |
| | Available | | | |
| | CLINICAL PRESENTATION † | CLINICAL PRESENTATION † | | |
| | 1) Suspected | N/A | | |
| | Opioid OD, | | | |
| | unresponsive | | | |
| | patient with | | | |
| | RAPID, | | | |
| | “thready” | | | |
| | pulse | | | |
| | indicating | | | |
| | LOW BP | | | |
| | 2) Suspected | N/A | | |
| | Opioid OD | | | |
| | with SLOW, | | | |
| | “thready” | | | |
| | pulse and | | | |
| | LOW BP | | | |
| | 3) Suspected | N/A | Droperidol § | |
| | Opioid OD | | (0.5 mg) | |
| | with | | 1-4 doses | |
| | prominent | | of BDC | |
| | RIGIDITY or | | | |
| | severe | | | |
| | laryngospasm | | | |
| | 4) Suspected | N/A | | |
| | Opioid OD is | | | |
| | pulseless-NO | | | |
| | PULSE and | | | |
| | LOW BP | | | |
| | pressure | | | |
| | POLYSUBSTANCE | POLYSUBSTANCE | | |
| | NON- | N/A | | |
| | MEDICAL | | | |
| | MEDICAL W/O | Flumazenil | | |
| | AIRWAY W/ | (0.2 mg) | | |
| | Hemodynamic | ** | | |
| | Monitoring | Dilantin | | |
| | avail. | (50 mg) | | |
| | | *** | | |
| | MEDICAL W | Flumazenil | | |
| | AIRWAY | (0.2 mg) | | |
| | W/ AW EQUIP | ** | | |
| | AVAIL. | Dilantin | | |
| | | (50 mg) | | |
| | | *** | | |
| | PROPHYLAXIS | PROPHYLAXIS | | |
| | IV USER | N/A | Droperidol § | |
| | | | (0.5 mg) | |
| | | | 1-4 doses | |
| | | | of BDC | |
| | 1^{st} | N/A | Droperidol § | |
| | RESPONDER | | (0.5 mg) | |
| | | | 1-4 doses | |
| | | | of BDC | |

With regard to Table 1:
† Uncertain Presentation: In the event that the medical provider is uncertain of physiologic or clinical presentation in “Suspected Opioid Overdose”, use the baseline “Non-Medical” dosing kit until vital signs are apparent and directional, then follow algorithm as above in clinical scenarios 1-4.
* All of these drug classes, with the exception of the A1ARAs prazosin and tamsulosin (see “A1ARA IV/IN/IM formulation protocol”), are available as IV formulations and therefore can be easily converted to nasal dosing regimens, which are similar in potency and concentration, if not the same, and will be concentratable in a nasal (e.g., insufflated), INH, IV, IM, IO and intraocular (IOC) formulation.
** Dilantin and Flumazenil are given in a ratio of 50 mg/0.2 mg as a prophylaxis against the risk or occurrence or seizures due to rapid benzodiazepine reversal in drug overdoses involving individuals with regular or habitual use of benzodiazepines.
*** In the event of “status epilepticus” induced by rapid reversal of benzodiazepine overdose, a conversion to use of separate baseline reversal drug (e.g. MU + NS-A1ARA + S-A1ARA) with IV Dilantin (5-15 mg/kg) with infusion rate NTE 50 mg/min due to risk of cardiac arrhythmia.
****Epinephrine is to be used with caution in individuals with F/FAs overdose due to the direct and potent activity of Epinephrine and Noradrenaline at the LC and FIMR related circuitry. However, should this be the initial presentation in “Suspected Opioid Overdose”, the medical practitioner should use their discretion to follow best practices and go directly to the most current ACLS treatment algorithms with the possible addition of the “Baseline formulation” for FIMR reversal.
§ Droperidol (combined alpha-1 adrenergic antagonist and anticholinergic-AARA-AC) can be administered in a dose range of 0.01-0.25 mg/kg IV, IM or IN. Dosing at higher ranges is known to be associated with increased risk of cardiac arrhythmias and is contraindicated in prolonged QTc intervals(SEE black box warning label), however is rare in occurrence. Initial doses of up to 2.5 mg are well tolerated with additional doses of 0.5-1.25 mg may be administered if benefit of F/FA overdose or toxic exposure reversal outweighs potential risk of upper dose range. Similarly, scopolamine may be used in a dose range of.
♦Pilocarpine (M3/muscarinic agonist) is an example of a muscarinic agonist recommended for use in events where it may be of benefit in F/FA overdose or toxic exposure and reversal of F/FA outweighs potential risk of upper dose range.
(NOTE:
Broad-acting muscarinic agents may in some cases provoke laryngospasm; development of M3-specific agonist may overcome this).

The following sections describe information and steps to support therapeutically effective treatments for preventing or reversing one or more effect(s) of opioid(s) or opiate(s) in an individual (for instance, to treat or prevent accidental overdose or to provide prophylaxis against environmental exposure). The sections include:
(i) Abbreviations & Exemplary Definitions;
(ii) Fentanyl and its Effects;
(iii) Proposed Mode(s) of Action
(iv) Therapeutic Compounds (including subsections (a) α1-Adrenergic Receptor Antagonists; (b) Mu receptor antagonist; (c) vasopressors; (d) anticholinergics; (e) paralytics/muscle relaxants; (f) centrally acting respiratory stimulants; (g) anti-seizure/membrane-stabilizing agents; (h) α2-adrenergic receptor agonist; (i) GABA/benzodiazepine receptor complex antagonists; and (j) Mu receptor agonists); (k) cholinergics (l) muscarinic agonist (m) nicotinic agonist
(v) Compositions for Methods of Use;
(vi) Methods of Use; and
(vii) Kits.

### (i) Abbreviations & Exemplary Definitions

A1ARs α1 Adrenergic receptors
A1ARAs α1 Adrenergic receptors antagonists
A1-A α1-A Adrenergic receptors antagonists—subtype specific antagonists
A1-B α1-B Adrenergic receptors antagonists—subtype specific antagonists
A1-D α1-D Adrenergic receptors antagonists—subtype specific antagonists
AARA α adrenergic receptor antagonist
AC anticholinergic drug (M1-M5 antagonists)
AW airway
BP blood pressure
C cholinergic drug (M1-M5 agonist, Nicotinic receptor agonist)
D5W 5% dextrose in sterile water
FIMR fentanyl induced muscle rigidity
FIRMR fentanyl induced respiratory muscle rigidity
F/FAs fentanyl and fentanyl analogues
HR heart rate
IRMAW Immediate Reversal Medical AW
IRMnAW Immediate Reversal Medical No AW
IRNM Immediate Reversal Non-Medical
M muscarinic receptors
M3 specific muscarinic receptor
M1-M5 muscarinic receptors,
NIC nicotinic receptors
PAOU Prophylaxis for Active Opioid User
PFR Prophylaxis for First Responders
PILO Pilocarpine
Poly Polysubstance
WCS wooden chest syndrome (combined FIRMR and laryngospasm)
VC vocal cords

The term “synergistic” as used herein means that the effect achieved with the compounds used together is greater than the sum of the effects that result from using the compounds separately. For example some of the compounds will include: mu or opioid receptor (mu, kappa, delta receptor subtypes) antagonists/agonists and α adrenergic antagonists, α adrenergic agonists, respiratory accelerants, vasoactive agents, anticholinergics, cholinergic agents (muscarinic receptor antagonist/anticholinergic, M3 receptor agonist or a nicotinic receptor general or selective agonist) and/or paralytics described herein are sometimes referred to herein as the “synergistic ingredients” or the “synergistic compounds.”

The degree of synergism of the combinations of the herein disclosed technology can be analyzed by estimation of a combination index (Fu et al., Synergy, 3(3):15-30, 2016). In some embodiments, the term “synergistic combinations” refers herein to combinations characterized by a combination index>1.

The term “synergistic combinations” refers herein to combinations characterized by an α parameter that is positive and for which the 95% confidence interval does not cross zero. In the practice of the present invention, the synergistic combinations preferably are characterized by an α interaction parameter that is greater than about 2, and more preferably by an α parameter that is greater than about 4.

The term “pharmaceutically acceptable derivative” is used herein to denote any pharmaceutically or pharmacologically acceptable salt, ester, amide or salt of such ester or amide of a synergistic compound according to the invention.

A “pharmaceutically acceptable salt” is intended to mean a salt that retains the biological effectiveness of the free acids and bases of the specified compound and that is not biologically or otherwise undesirable. Examples of pharmaceutically acceptable salts include but are not limited to sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caprotes, heptanoates, propioltes, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, sulfamates, xylenesulfonates, phenylacetates, phenylpropionates, phenylbutyrates, citrates, lactates, gamma-hydroxybutyrates, glycollates, tartrates, methanesulfonates, propanesulfonates, naphthalene-1-sulfonates, naphthalene-2-sulfonates, and mandelates.

“Analogs” is intended to mean compounds derived from a particular parent compound by straightforward substitutions that do not result in a substantial (i.e. more than 100×) loss in the biological activity of the parent compound, where such substitutions are modifications well-known to those skilled in the art, e.g., esterification, replacement of hydrogen by halogen, replacement of alkoxy by alkyl, replacement of alkyl by alkoxy, etc.

“Therapeutically effective combination” means an amount of a compound herein described combination that, when administered to a patient in need of treatment, is sufficient to effect treatment for the disease condition alleviated by the (synergistic) combination. In the immediate reversal scenario, several metrics are significant in monitoring for successful treatment. A combination drug is beneficial as no single agent treats all three of the active receptor sites engaged by fentanyl and other F/FAs: mu opioid receptors, muscarinic and alpha adrenergic receptors. Particularly, naloxone has a minimal impact on the effects of F on VC and laryngeal muscles/laryngospasm at doses relevant or safe to humans (e.g. naloxone effect at >0.8 mg/kg in rat model) (Willette et al., J Pharmacol Methods 17:15-25, 1987; Willette et al., Euro J Pharmacology 80:57-63, 1982; Willette & Sapru, Euro J Pharmacology 78:61-70, 1982).

Several broad categories of therapeutically effects exist, including:
1) Attenuation or Resolution of FIRMR or WCS: measured by a reduction, elimination or inhibition of chest wall rigidity, diaphragm rigidity, laryngospasm with return of airway patency and either easy flow of oxygen and ventilation with assisted ventilation or the return of spontaneous respiration with adequate respiratory rate and tidal volume to maintain oxygenation (e.g. Oxygen saturation of >94% by pulse oximetry, Arterial Blood gas-ABG with P-arterial O₂ of >80 mmHg pressure of oxygen in the blood PaO₂ ETCO₂<40).
2) Return of consciousness and able to follow commands with Glasgow Coma scale score of >12 (8=comatose but responsive to painful stimuli, 3=unresponsive to all stimuli).
3) Hemodynamic parameters adequate to maintain cerebral and coronary perfusion; usually Systolic BP>90 mmHg and Diastolic BP>50 mmHg, HR>50 BPM.
4) Similar or same parameters can be used for prophylaxis users. The prophylaxis agents essentially use a pre-emptive blockade/antagonism of Mu and Alpha adrenergic receptors to increase the dose tolerance and resistance to FIRMR or WCS upon exposure. The agents technically cause a R shift of the dose response curve for FIRMR or WCS and thus either inhibit or delay the onset of effects of F/FA exposure or allow for a higher level of exposure before effects occur.

Amounts of each of these components present in a therapeutically effective combination may not be therapeutically effective when administered singly. Use of the combination is important because no single agent treats all three of the active receptor sites engaged by fentanyl and other F/FAs, notably mu opioid receptors, muscarinic and alpha adrenergic receptors. For instance, naloxone has a minimal impact on the effects of F on VC and laryngeal muscles/laryngospasm as noted above in doses relevant to or safe for humans (Willette et al., J Pharmacol Methods 17:15-25, 1987; Willette et al., European Journal of Pharmacology 80:57-63, 1982; Willette & Sapru, European Journal of Pharmacology 78:61-70, 1982) The amount of a given combination that will be therapeutically effective will vary depending on factors such as the particular combination employed, the particular form of opioid/opiate exposure, the treatment history of the patient, the age and health of the patient, and other factors.

“Treatment” in some instances refers to alleviation or prevention of symptoms of FIRMR or WCS and respiratory depression in a patient or the improvement of FIRMR or WCS in an individual in need of such treatment. However, “treatment” in the context of this disclosure is several fold, depending on the embodiment(s):

1. Immediate reversal of FIRMR or WCS and respiratory depression: The most basic intervention level (e.g., mu antagonist and AARA) for FIRMR or WCS reversal results from the antagonism or blockade of mu receptors, or opioid receptor (mu, kappa, delta receptor subtypes) antagonist combined with an α adrenergic antagonist/agonist to decrease noradrenergic outflow from the LC triggered either directly or indirectly at mu opioid or α adrenergic receptors by F/FAs. Additionally a cholinergic agent (muscarinic receptor antagonist/anticholinergic, M3 receptor agonist or a nicotinic receptor general or selective agonist) may be optionally added to antagonize the potential direct or indirect effects of fentanyl and F/FAs on muscarinic receptors and nicotinic receptors. This can be gauged as mentioned previously by either the return and ease of spontaneous respiration or the return of ability to perform assisted ventilation and/or the ability to secure the AW if necessary. Essentially this is a return of AW patency and increase in thoracic compliance (e.g. relaxed chest wall muscles) that allows for oxygen exchange and the reversal of hypoxemia and hypercarbia and can be objectively measured by end-tidal-CO₂ concentrations (ETCO₂), Pulse oximetry (O₂ Saturation % difference between oxygenated hemoglobin—Hgb and deoxygenated Hgb) and arterial blood gas concentrations (PaO₂, PaCO₂ in mmHG). Return of level of consciousness (LOC) using the Glasgow Coma Scale as noted above, is also a significant measure of the reversal of is routinely associated with an instantaneous loss of consciousness with return of consciousness as FIMR is wearing off or actively inhibited.

2. Prophylaxis against FIMR: This can be gauged by the either the prevention of FIMR or a reduction in AW and ventilation compromise symptoms upon contact exposure to F/FAs in the environment. This can be measured objectively by the dose-response curve or concentration of F/FAs that induce rigidity and mechanical compromise of the AW. If the treatment is effective it will shift the dose response curve to the right meaning that it will take more of F/FAs at a given concentration to cause FIRMR or WCS. Ideally, if the NA outflow from the LC has been previously inactivated by an AARA, which acts to hyperpolarize and inactivate NA neurons in the brainstem and spinal cord and blocks norepinephrine from landing on AARAs, even exceedingly high doses of F/FAs and contact exposure may allow the patient to remain asymptomatic or only mildly affected. The combined effect of a long acting Mu opioid antagonist (e.g. naltrexone, nalmefene) and an alpha 1 adrenergic antagonist are ideal for prophylaxis. In the case of individuals who are affected despite receiving a prophylaxis dose, an immediate reversal dose can be “stacked” on top of the prophylaxis dose to block and or antagonize any of the remaining receptors that might still be available for binding by F/FAs.

3. “Stacking dose”: in the event that an individual has already received prophylaxis dosing, but becomes symptomatic in a F/FAs contaminated environment, additional doses of the immediate reversal agent can be given. In this case it may be recommended to give a modified version of the immediate reversal agent that includes Naloxone, a 1A or 1D subtype selective AARA (e.g., tamsulosin) and a vasoactive agent (e.g., phenylephrine or ephedrine). Additionally, a cholinergic agent (muscarinic receptor antagonist/anticholinergic, M3 receptor agonist or a nicotinic receptor general or selective agonist) optionally may be added to antagonize the potential direct or indirect effects of fentanyl and F/FAs on muscarinic receptors and nicotinic receptors in the presentation of significant vagal tone demonstrated clinically as bradycardia (HR<60 BPM). Similar parameters can be used to measure success of reversal as mentioned above in this section.

### (ii) Fentanyl and its Effects

First developed by Janssen Pharmaceuticals in the 1950's as a more hemodynamically stable and potent analgesic alternative to morphine and other synthetic opiates, fentanyl and its analogues (FAs) are highly potent, synthetic, mu-opiate receptor agonists with a potency 100-10,000 times greater than morphine or heroin. Despite having a very narrow therapeutic window, the fentanyl family of opioids have been safely used in medicine for over 50 years and to great effect in surgical anesthesia and pain management, when administered by Anesthesiologists and trained medical personnel (Grell et al., Anesth Analg 49(4):523-532, 1970; Streisand et al., Anesthesiology 78(4):629-634, 1993; Bennett et al., Anesthesiology 87(5):1070-1074, 1997; Coruh et al., Chest. 143(4):1145-1146, 2013).

Naloxone, a mu opioid receptor antagonist, is currently the only FDA-approved medication for reversal of opioid overdose and specifically targets respiratory depression induced by opioids. Recent public health reports from major urban areas affected by increasing numbers of overdoses involving fentanyl and its analogues have reported a dramatic rise in the number of naloxone doses needed to reverse the effects of fentanyl (e.g. 2-12 doses of naloxone; Walley et al., Morb Mortal Wkly Rep 66:382-386, 2017; Chou et al., Ann Intern Med 167(12):867-875, 2017). High dose naloxone (e.g. 0.2 mg/kg), and even doses that are two times the normal dose (e.g. 0.0005 mcg/kg)), regularly precipitate severe cardiac arrhythmias, hemodynamic instability and pulmonary edema in active opioid users, which are all potentially life-threatening (Clarke et al., Emergency Med 22:612-616, 2005). Animal models have demonstrated that naloxone has a minimal effect on vocal cord closure and the upper AW effects of fentanyl in dose ranges relevant to or safe for humans (Willette et al., J Pharmacol Methods 17:15-25, 1987). The mechanism/s of these vocal cord and upper AW effects have not been identified.

Naloxone's effectiveness for reversing fentanyl overdose is possibly limited due to fentanyl's unique potency and binding at non-opiate receptors and/or non-opiate receptor distributions in the brainstem and other regions that control motor efferent output to the chest wall, larynx, vocal cords and respiratory diaphragm. Inappropriate activation of these receptors by fentanyl results in respiratory muscle rigidity and airway paralysis (Fu et al., Anesthesiology. 87(6):1450-1459, 1997; Lui et al., Neurosci Lett. 201(2):167-170, 1995; Milne et al., Can J Physiol Pharmacol. 67(5):532-536, 1989; Lui et al., Neurosci Lett. 108(1-2):183-188, 1990; Lui et al., Neurosci Lett. 96(1):114-119, 1989; Sohn et al., Anesthesiology 103: 327-334, 2005; and Root-Bernstein et al., Int J Mol Sci. 19(1), 2018). Fentanyl has a similar binding affinity (Ki) at mu-opioid receptors as morphine and the leading antagonist drugs used to reverse opioid overdose (e.g. naloxone; Evers, Maze & Kharasch. Anesthetic Pharmacology. Cambridge University Press, 2011; Volpe et al., Regul Toxicol Pharmacol 59(3):385-390, 2011; Clarke et al., Emergency Med 22:612-616, 2005). Given this similar binding affinity of morphine and fentanyl and the fact that naloxone has a greater binding affinity at mu opioid receptors, it is surprising clinically, that fentanyl overdose requires repeated doses of naloxone to reverse its specific effects (Walley et al., Morb Mortal Wkly Rep 66:382-386, 2017; Clarke et al., Emergency Med 22:612-616, 2005; Chou et al., Ann Intern Med 167(12):867-875, 2017). This is a key indicator of fentanyl binding at receptor sites other than the opiate/mu receptors and that FIRMR and/or WCS has a limited relationship to mu receptor activation that has not been fully described to date.

### (iii) Proposed Mode(s) of Action

The following discussion is provided for context, is based on the knowledge, experience, and professional expertise of the inventor, but in no way is it intended to limit the function or practice of the technology and discoveries described herein. Described herein are proposed pharmacological mechanisms specific to F/FAs and facilitate development of more effective treatments for F/FA overdose and toxic exposure. Prior to this disclosure, there remain several critical issues and/or gaps in the basic knowledge of the underlying mechanisms of F/FA-induced WCS, including: 1) the false perception that F/FA's effects are similar to morphine-derived opioids, but more potent, and are therefore treatable simply with higher doses of MOR antagonists; 2) current public health data clearly indicate that naloxone is not effective for F/FAs, but there has been little new drug development; 3) previous work with animal models of WCS (e.g., Jerussi et al., Pharmacol Biochem Behav, 28(2):283-289, 1987; Lui et al., Neurosci Lett, 157(2):145-148, 1993; Lui et al., Neurosci Lett, 96(1):114-119, 1989; Lui et al., Neurosci Lett, 108(1-2):183-18, 1990; Lui et al., Neurosci Lett, 201(2):167-170, 1995; Weinger et al., Brain Res, 669(1):10-18, 1995; Yang et al., Anesthesiology, 77(1):153-161, 1992) occurred prior to human studies demonstrating the involvement of vocal cords (VC) in human F/FA induced WCS (Bennett et al., Anesthesiology 87(5):1070-1074, 1997). No animal model since has incorporated this VC effect to further explore WCS from F/FA's and prior models bypassed VCs with either endotracheal intubation or tracheostomy, creating a years-long gap in the literature. Therefore, the effects of potential therapies on VC function and upper airway mechanical failure from F/FAs have been unknown. The precise mechanism of action (MOA) whereby fentanyl increases noradrenaline (NA) outflow from the Locus coeruleus (LC) is still unknown and has not been demonstrated, although several MOA have been suggested.

Fentanyl has a significant binding affinity to α-1B adrenergic receptor subtypes, with a rank binding order of 1B˜1A and (1:5)>1D (e.g. 1 B˜1 A>>1 D) and has been shown to act as an antagonist at these receptor subtypes. Given that other A1ARAs (e.g. prazosin, tamsulosin) block the effects of norepinephrine (NE) at these receptors and limit NA outflow from the LC, it is difficult to imagine that F/FAs may not have similar effects. Thus, an antagonist at alpha 1 adrenergic receptors would be expected to limit noradrenergic effects.

Another MOA that the inventor will suggest and not found in the current literature, may be that because fentanyl binds and antagonizes receptor subtypes A-1A and A-1B, but has a 5 fold less binding affinity for the A-1D adrenergic receptor subtype, may allow for unopposed or facilitated agonism, activation, or stimulation of A-1D adrenergic receptors by NE. The NE that is being released in the LC may be caused by fentanyl binding to mu opioid receptors, mu opioid receptors on GABA interneurons, cholinergic receptors and/or some combination of these receptors. Regardless of the MOA, unopposed agonism of isolated α-1 adrenergic receptors could result in profound systemic hypertension from arterial contractility, decreased blood flow/decreased hepatic perfusion and a rapid increase in motor output to the muscles of respiration and the larynx and vocal cords/AW. It is important to note that these are only some of the possible MOA, most of which have not been suggested or discussed in the literature. Additionally, it is not the intent of this document to be a complete, comprehensive or exhaustive review of all the possible MOAs suggested for FIMR or to be limited in scope by the MOA mentioned here.

Although the MOA of F/FAs is ill-defined and not completely understood, the existing animal data suggests (Fu et al., Anesthesiology. 87(6):1450-1459, 1997; Lui et al., Neurosci Lett. 201(2):167-170, 1995; Milne et al., Can J Physiol Pharmacol. 67(5):532-536, 1989; Lui et al., Neurosci Lett. 108(1-2):183-188, 1990; Lui et al., Neurosci Lett. 96(1):114-119, 1989; Sohn et al., Anesthesiology 103:327-334, 2005; and Root-Bernstein et al., Int J Mol Sci. 19(1), 2018) that fentanyl and its analogues (such as sufentanil, alfentanil, remifentanil, and carfentanil) have the ability to bind to (that is, associate specifically with) Mu opioid receptors in the LC of the Pons/brainstem. Through an unclear mechanism that has been only partially explored, fentanyl and its analogues cause increased NA flow from the LC and via spinal motor neurons and sympathetic fiber tracts to the muscles of respiration and the intrinsic muscles of the airway, cause fentanyl induced muscle rigidity in animals (FIRMR and/or WCS in humans) and life-threatening, mechanical failure of the respiratory system and in some cases the cardiovascular system. However, the upper airway effect of laryngospasm have not been studied in the animal model even though laryngospasm is the key feature of WCS in humans. Conversely, the neuropharmacologic mechanisms underlying WCS have not been studied in humans. This MOA is difficult to explain because it contradicts the general medical and scientific pharmacologic consensus regarding the action of opiates on the sympathetic nervous system (e.g. opiates/opioid receptor antagonists consistently depress NA neuronal output and sympathetic outflow from the CNS).

This is a mechanism that is poorly understood and difficult to reconcile with the well-established medical and scientific literature that supports that all opioids, including F/FAs reduce catecholamine levels in the CNS and peripheral nervous systems specifically norepinephrine levels (Aghajanian, The Journal of Clinical Psychiatry 43:20-24, 1982) and the fact that fentanyl acts as an antagonist at all 3 of the alpha1 adrenergic subtypes (Sohn et al., Anesthesiology 103:327-334, 2005) similar to other alpha1 adrenergic antagonists (prazosin and terazosin) yet the reaction of F/FA induced FIMR does not occur without noradrenergic activation of the LC and conversely is completely inhibited by the administration of high dose alpha1 adrenergic antagonist agents. The doses of prazosin used are in a high dose range that would be lethal to humans, making the information unusable, therefore a better more detailed elucidation and understanding of the mechanism will be required to design safe and effective therapy for the FIRMR effects of WCS. Another significant limitation in the previous work/studies is that the effects of this therapy on VCC laryngospasm was not studied or evaluated. It has been suggested that naloxone (mu antagonism) is not effective for preventing VCC in this model (Willette et al., J Pharmacol Methods 17:15-25, 1987) and unclear whether alpha adrenergic antagonism would be effective since the sole innervation of the laryngeal muscles is controlled by the parasympathetically dominant vagus nerve. Vagal motor neurons are more likely to involve cholinergic innervation based on the parasympathetic tone via vagal nerve fibers to the laryngeal muscles which controls all intrinsic muscles of the larynx. The most effective treatment for laryngospasm may involve the modulation of cholinergic motor neurons with muscarinic receptors (M1-M5), although this has not been demonstrated in the animal model. The fact that fentanyl may act as an antagonist at M3 receptors may also facilitate selective binding of Ach at the M1 M2 M4 receptors and facilitate activity of the laryngeal muscles. This will be established by using competition binding assays with F and Ach at M (M1-M5) receptors to note activity and then use of an animal AW model to demonstrate the efficacy of these combined treatments.

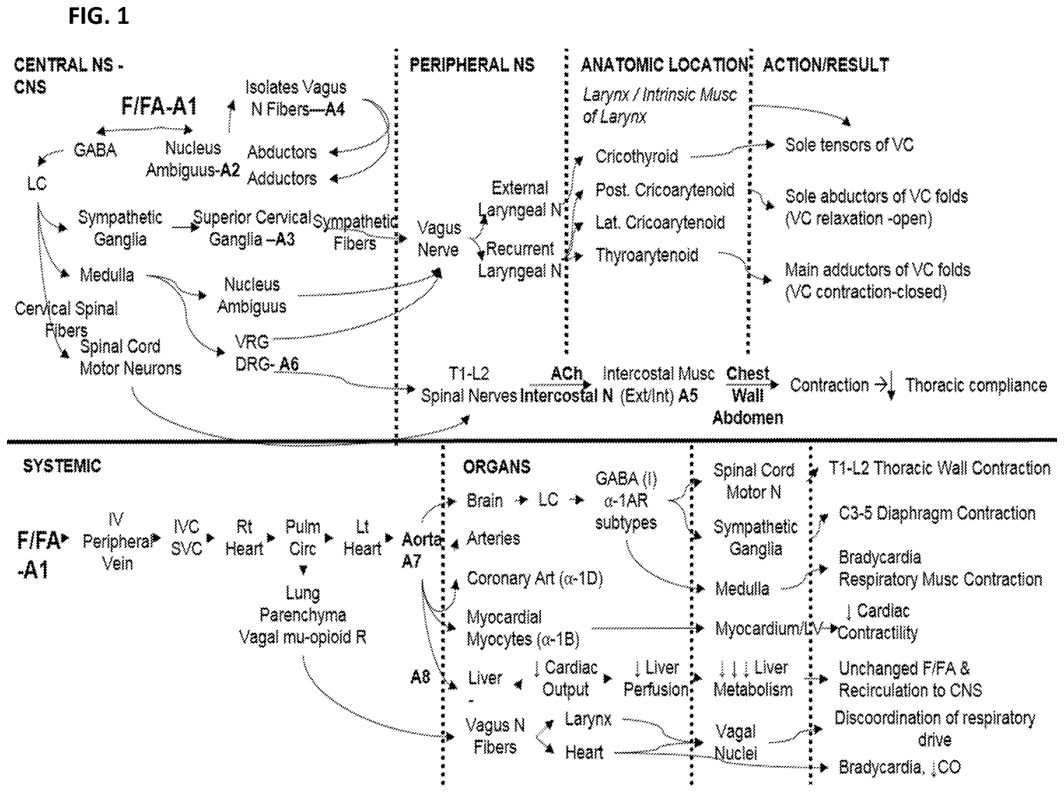

The LC is a key component or target in the treatment of FIMR, because it has the highest concentration of noradrenergic neurons in the entire mammalian CNS, is the major production site of noradrenaline in the CNS, and the key nexus communicating with medullary and pontine respiratory nuclei controlling afferent and efferent motor control to the muscles of respiration including the larynx and vocal cords. It has neural fibers that run to and provide noradrenergic input to nearly all major structures in the brain including the cortex, thalamus, amygdala, and the raphe nucleus and to the centers in the brainstem such as the medulla, spinal motor neurons, and to the ventral and dorsal horns (VH, DH) of the spinal cord (FIG. 1). The DH and VH are primarily and densely populated with NA neurons and α adrenergic receptors. Stimulation of these α adrenergic receptors with NA either experimentally or in vivo results in excitation and contraction of terminal sites on skeletal muscle fibers located in the chest wall, abdominal wall and diaphragm (Fu et al., Anesthesiology. 87(6):1450-1459, 1997; Lui et al., Neurosci Lett. 201(2):167-170, 1995; Milne et al., Can J Physiol Pharmacol. 67(5):532-536, 1989; Lui et al., Neurosci Lett. 108(1-2):183-188, 1990; Lui et al., Neurosci Lett. 96(1):114-119, 1989; Sohn et al., Anesthesiology 103: 327-334, 2005; and Root-Bernstein et al., Int J Mol Sci. 19(1), 2018). In the case of the sympathetic neurons, this takes the dermatomal and spinal nerve distribution of vertebral levels T1-L2, which maps to the thoracic/chest wall, abdominal muscles, and part of the diaphragm in humans (FIG. 1). If the chest wall muscles get contracted in a large volume or maximal inspiration via the external intercostal muscles, this can trigger afferent signals from “stretch” or “J” receptors in the lung parenchyma and chest wall that then go back to the Dorsal Respiratory Group (DRG) and Ventral Respiratory Group (VRG) groups of neurons located in the major respiratory center in the medulla region of the brainstem via the vagal nerves. This is known as “the “Hering-Breur reflex” arc. Activation of the DRG and VRG or activation via these reflex arc results in increased excitability of the efferent motor neurons with the end result being skeletal muscle contraction in the external intercostal muscles of the chest wall, abdominal wall and diaphragm and increased contractility of the larynx and closure of vocal cords (FIG. 1). Similarly, increased NA outflow from the LC can travel to sympathetic innervation of the vocal cords via superior cervical ganglia and the vagal fibers innervating the laryngeal muscles, mediating adductor activation and/or abductor relaxation resulting in laryngospasm.

As an Anesthesiologist in clinical practice for more than 20 years, I have administered fentanyl and fentanyl analogues to more than 20,000 patients, amounting to several hundred thousand doses. I have clinically treated FIRMR and/or WCS on a number of occasions, and my years of clinical experience and knowledge from having seen and treated this phenomenon first hand have provided me with a unique perspective and clinical insight into the underlying molecular mechanism of WCS that resulted in the discoveries described herein.

In addition to my clinical observations in Anesthesia, I have worked and trained extensively as an Addictionologist and have been able to further consolidate and confirm my knowledge of FIRMR/WCS from a hundred or more eyewitness accounts and interviews with survivors of fentanyl overdose or witnesses to F/FA overdose deaths. From these accounts, I was able to correlate my clinical observations and treatment of WCS as an anesthesiologist with the clinical presentations of F/FA overdose and would conclude that the underlying mechanism of death in F/FA is actually WCS. The consistency of the clinical presentations described and my clinical experience with WCS has given me the knowledge and skill to develop treatments accordingly and as described here and teach their implementation to the public.

One blinded case study arose from a public discussion I had with an individual who was not a patient of mine (and with whom I have no personal or professional relationship). Despite that individual having limited to no medical knowledge or knowledge of wooden chest syndrome, they provided the detail of an overdose with a sub-lethal dose/known quantity of fentanyl and effectively described in what I can only call “textbook detail”, the engagement of the external intercostal muscles in a maximal inspiratory position and acute vocal cord (VC) closure that was persistent for approximately three minutes before a loss of consciousness (LOC) occurred.

Based on my own clinical observation in Anesthesia and Addictionology, and having treated WCS in human subjects on a number of occasions, I observed that the main respiratory muscles involved in the reaction of FIRMR appear to be the intercostal muscles of the chest wall which are used specifically in respiratory inspiration and expiration. I have also observed laryngospasm immediately after a 3 mcg/kg dose of fentanyl that was resistant to succinylcholine that required an emergent cricothyrotomy and transtracheal jet ventilation to restore oxygenation.

A single study (Sohn et al., Anesthesiology 103:327-243, 2005) showed that fentanyl could bind to the A-1B, A and 1D adrenergic receptors as an antagonist in isolated segments of canine pulmonary artery with such affinity that it could competitively block the potent effects of the α1B agonist, phenylephrine (e.g., phenylephrine has similar binding capacity to Noradrenaline at the alpha-1B receptor subtype) at concentrations [microM/10⁻⁶M] which are thought to be within the range of the therapeutic serum/tissue levels and concentrations of fentanyl (e.g. 10-25 ng/ml approximates a 10⁻⁷M (Yamanoue et al., Anesthesia & analgesia 76:382-390, 1993) and 2.96×10⁻³M for brain lipid (Stone & DiFazio, Anesthesia & analgesia 67:663-666, 1988; Sohn et al., Anesthesiology 103:327-334, 2005) concentration in the CNS) that are also found on autopsy from deaths caused by fentanyl and FAs. However, the binding affinity values were not clearly described in the study itself and cannot be compared directly to norepinephrine binding affinity values, as there are no clear values available from current scientific literature.

In turn, each of the α-1 adrenergic antagonists has a unique binding distribution at the α-1 subtypes. For example, the selective agent Tamsulosin has a 12-30-times greater binding affinity at the 1A subtype over other α-1 antagonists and greater binding affinity than Prazosin. Tamsulosin has similar potency at the 1D subtype. As a result of its subtype specificity, Tamsulosin has a lower impact on blood pressure compared to the non-selective agents such as Prazosin. Both agents have the ability to cross the blood brain barrier and thus can bind to α-1 receptors in the pons and LC. Thus, one embodiment provides a strategy to mitigate effects on hemodynamics/blood pressure by combining both agents (at a selected ratio, such as 1:1, 2:1, 3:1 in favor of the α1A selective agent) to allow for a decrease in hypotensive side effects (e.g. “first dose effect”) while optimizing antagonism of α-1 subtypes with each agent. In this case, Tamsulosin binds 1A and 1D subtypes while Prazosin is able to bind 1B adrenergic receptors at a dose that is lower than if prazosin were used as a single agent. This strategy allows for optimal antagonism of FIRMR/WCS while limiting the side effect profile of the non-selective agent Prazosin. This strategy is discussed further below.

Although the medical literature has described vocal cord-(VC) spasm/laryngospasm with FIRMR, the underlying molecular mechanisms have not been described in humans and the available animal data makes no observations of the effect of F/FA on the upper airway (e.g. larynx, vocal cords). The inventor's direct clinical observation that spasm of the VC was not immediately relieved by the muscle paralytic-succinylcholine, which acts in the periphery of skeletal muscle acetylcholine receptors (AchRs) suggests that F/FAs effects on the larynx and vocal cords is a centrally-mediated effect that may come from the LC, pontine (pons) and medullary (medulla) circuitry, as described above. The pathway for VC spasm/laryngospasm may come from several mechanisms such as direct activation of motor efferents in the medulla (e.g. VRG neurons, nucleus ambiguus) by way of NA neurons from the pons/LC or directly at cholinergic receptors in medullary nuclei by F/FAs themselves. Some studies also suggest that NA activation in the pons/LC may be mediated via increased ACH release into the LC by surrounding cholinergic nuclei and serves to increase NE release in the LC.

The literature prior to the priority date of this application adds no clear explanation or complete picture of the mechanism of action of fentanyl or FAs in WCS and FIRMR, particularly at the level of the alpha-1 adrenergic subtypes or at cholinergic receptors and the MOA remains unclear and non-obvious. Additionally, a significant limitation of the prior literature is that doses used in prior animal experiments were not meant to induce or increase human survival rates from WCS or FIRMR in F/FA overdose, but simply to demonstrate a possible MOA for FIMR. None of the animals survived those experiments. As such, the doses of α1-adrenergic antagonists used in those animal experiments would be routinely fatal to a substantial portion of subjects given such doses. Thus, the previously available animal data could not be used to develop therapeutics without significant modification, as taught for the first time herein.

The results in prior animal experiments were obtained through the use of dosing strategies that would cause significant mortality and morbidity in human subjects and thus, dosing levels presented in the animal data are unfeasible in humans without a significant modification in the side effect profile, the use of molecules that demonstrate significant synergy and/or a clearer understanding of the underlying mechanism of WCS and FIRMR in humans. In addition, other cholinergically mediated mechanisms of laryngospasm in WCS remained unexplored in animals and humans, that is until the pre-clinical development of this invention.

Dosing strategies using α1 agents that are appropriate to human subjects have not been explored until now, with the provision herein of combinations for therapeutic compounds to treat WCS/FIRMR in F/FA overdose or toxic exposure.

The goal here is to use either synergy between molecules, alleviate side-effects and/or improve/diminish the side effect profile of prazosin (e.g. severe orthostatic hypotension, syncope, life-threatening or severe hypotension, myocardial ischemia) to make treatment of WCS/FIRMR feasible in humans and is the key to being able to use this technology to improve the survival rate of F/FAs overdose.

Example treatments and methods described herein take advantage of and/or utilize the unique α-1 adrenergic receptor subtype binding affinities of different α-1 adrenergic antagonists, so as to optimize α-1 subtype antagonism while minimizing α-1 antagonist side effects (Including the primarily life-threatening hypotension that occurs with the non-selective agents). A combination of selective and non-selective α-1 antagonist agents is an exemplary dosing strategy to maximize receptor antagonism while minimizing mortality and morbidity from severe vascular and hemodynamic instability or compromise.

Thus, provided herein are dosing strategies using combinations of α-1 adrenergic receptor antagonist(s) and one or more other supportive agent(s) to minimize side effects and optimize survival and outcomes from WCS/FIRMR and overdose related to F/FAs and other opiates tainted with F/FAs.

### (iv) Therapeutic Compounds

Provided herein are pharmaceutical compositions, as well as methods of their use. Generally, these compositions include one or more of a therapeutically effective amount of α1-adrenergic receptor antagonist, in some embodiments in combination with a therapeutically effective amount of one or more of a Mu or opioid receptor subtype antagonist and/or a cholinergic agent (muscarinic antagonist/M3 agonist and/or nicotinic agonist) and/or a centrally-acting or peripherally acting respiratory stimulant and/or a GABA/benzodiazepine receptor complex antagonist, and in certain embodiments an α1-adrenergic receptor agonist and/or a Mu or opioid receptor subtype agonist, long-acting Mu or opioid receptor subtype antagonist, vasoactive/vasopressor agents for blood pressure support, anticholinergic agents, a centrally-acting α adrenergic receptor antagonist combined with a peripherally acting α adrenergic receptor antagonist, muscle paralytic and anticonvulsant or membrane-stabilizing agents. Optionally, the composition also includes a pharmaceutically acceptable carrier, such as lipophilic agents or nano-particle technology or other carriers discussed herein and/or known in the art for delivery as IV, IM, INH, IO, PO etc. For instance, eye drops (IOC delivery) is a simple method of drug administered that can be used to effectively deliver agents in to the CNS, as the eye is an extension of the CNS itself. IOC may represent a particularly beneficial route of delivery to the CNS, given that pilocarpine (M3 agonist) and atropine are and can readily be administered as eyedrops in the case of anticholinergic or cholinergic treatment. Similarly, inhaled (INH) delivery can be used, for instance for prophylaxis, in a nebulizer, metered-dose inhaler (MDI), or as a vaping or vaporization INH solution. Reversal compositions can be delivered via INH routes, if the airway is patent or delivery made through an endotracheal tube.

Mu or opioid receptor subtype antagonists are used herein for alleviating or inhibiting the dose dependent respiratory depression caused by all opiates/opioids and any intermediary effects leading to activation or antagonism of other receptor subtypes (e.g. GABA interneurons, alpha adrenergic receptors, cholinergic receptors). Short duration and rapid acting agents (e.g., naloxone, narcan, nalmefene) are used for immediate reversal, while longer acting agents (e.g., naltrexone) can be used for prophylaxis.

Alpha adrenergic receptor antagonists (AARAs) are used herein to inhibit WCS/FIRMR. In various embodiments, selective or non-selective antagonists or combination agents (e.g. alpha adrenergic antagonist and anticholinergic antagonist, such as droperidol) are used either singly or in combination to minimize the effects of AARAs on blood pressure and will use delivery into the CNS via nasal insufflation to minimize the peripheral effects of AARAs on blood pressure. In addition, AARAs will be used in combination with vasoactive agents as noted above to offset, counteract or minimize the effects of the unfavorable effects of AARAs on blood pressure and hemodynamics. This would be particularly helpful at times of overdose resuscitation since most patients will be hemodynamically depressed. These combinations can be used in either immediate reversal agents or in prophylaxis compounds.

Anticholinergic agents can be used herein, in patients who are either bradycardic or asystolic, to decrease vagal tone (baseline heart rate) or to alleviate cholinergically mediated closure of vocal cords/laryngospasm in patients who are using these drugs for prophylaxis or immediate reversal.

Respiratory accelerants can be used in the immediate resuscitation scenario to synergistically impact and reverse the inhibitory effects of opiates/opioids on the CO₂ and O2 chemoreceptors located in the carotid body, aortic body and possibly the heart. Opiates depress respiratory drive by depressing the reactivity and response of these chemo-sensors to increase respiratory drive in the face of increasing serum levels of CO₂ and/or decreases in levels. This is inhibition of hypoxia driven respiratory drive is a significant way that opiates cause hypoxemia in opioid overdose. Other cholinergic agonists (e.g. nicotine) or antagonists with activity on central respiratory neurons (e.g. pontine Kölliker-Fuse neurons) can also be used in combination with these respiratory accelerants. To be used in immediate resuscitation scenarios.

Muscle relaxants and paralytics (such as succinylcholine and rocuronium) are rapid acting, and optionally can be used as described herein to alleviate WCS/FIRMR and overdose related to F/FAs particularly in chest wall and diaphragm and may help to relieve spasm of the vocal cords and larynx. Although low doses on the order 1-3 mg for Succinylcholine can be used to decrease the WCS/FIRMR and overdose related to F/FAs, it is preferable to use full intubation doses (e.g. 1-1.5 mg/kg) to secure the airway with an endotracheal tube. These drugs would be used in immediate resuscitation scenarios by individuals who are trained in invasive AW management.

Similarly, GABA receptor complex agents can be added to these compounds for overdoses involving opiates, F/FAs and benzodiazepines. Similarly, an anticonvulsant such as Dilantin can be added to this compound to act as prophylaxis against seizures that can occur in the rapid reversal of benzodiazepines in long term or habitual users of benzodiazepines.

It is contemplated that the therapeutic agents can be administered to a subject (for instance, a subject in need of prevention or reversal of one or more effect of an opiate or opioid compound) at the same time, or in sequence/series, in various embodiments and with various durations of onset and action as described herein. In embodiments that contain two (or more) different therapeutic compounds (that is, combination formulations or combined therapeutics), optimally the pharmaceutical composition includes a set proportion or proportion range of one therapeutic compound to another in the composition. Some examples would include, a combined therapeutic in some embodiments with a ratio of 0.5-1 parts naloxone to 1 parts prazosin; and/or (b) a ratio of 0.1 parts AARA to 1-20 parts Phenylephrine; and/or (c) a ratio of 0.1 parts AARA to 10 parts ephedrine. These exemplary ratios are on the higher side of the dosing range and can be scaled lower and are not meant to be a complete or limiting description here of all the ratios that can be effectively utilized. Additional description of compounds useful for the compositions and methods described herein are discussed below.

The disclosure provides a platform of compounds and molecules that either singly or in combination block/antagonize/modulate or prophylax against the effects of piperidine derived opioids (e.g. fentanyl and fentanyl analogues) effects on the neurophysiology and mechanics of respiration, with the addition of one or more other molecules to either synergize reversal of F/FAs overdose or offset side effects of dose requirements required for optimal treatment. The platform also includes the use of F/FAs in combination with an A1ARA to optimize analgesia with prophylaxis against WCS/FIRMR.

The following are descriptions of representative compounds that are applicable to be used in one or more of the therapeutic combination treatments provided herein. Many of these compounds have recognized, well-known safety profiles and dosing strategy guidelines, though guidance is provided herein. VIVITROL® (naltrexone for extended-release injectable suspension) and Nasal NARCAN® (naloxone hydrochloride) are listed below as examples of industry acceptable delivery methods for intramuscular (IM) extended-release injectables and formula solutions for nasal insufflation, respectively. Dosing charts provided herein supply an abbreviated summary of dosages and practitioner guidelines for the use of representative product(s)/compound(s) as is suitable for the clinical presentation requiring treatment.

### (a) α1-Adrenergic Receptor Antagonists

α-1 adrenergic receptor blockers inhibit vasoconstriction by blocking norepinephrine binding to α-1 post synaptic membrane receptors, which inhibits the blood vessels from contraction and can block norepinephrine effects centrally in the LC. It happens because a 1 blockers inhibit the activation of postsynaptic α-1 receptors and prevent the release of catecholamines (Sica, J Clin Hyperten. 7(12):757-762, 2005). α-1 adrenergic receptor antagonists block a receptors and relax the smooth muscles in the vascular system and bladder. Alpha-1 blockers lower blood pressure by blocking α-1 receptors so norepinephrine can't bind the receptor causing arterial vessels to dilate. In view of these vascular effects, selective α-1 blockers are better tolerated than non-selective α blockers, due to less hypotension. Terazosin, tamsulosin and doxazosin are prime drugs prophylaxis because they have a long half-life and modified release formulations and have selectivity for alpha 1D receptor subtypes. Tamsulosin is particularly ideal because it minimally affects the blood pressure and the side effects of vasodilation is minimal compared to less selective agents (prazosin) (Kaplan, Am J Med. 80(5B):100-104, 1986). See also Yoshizumi et al. (Am J Physiol Renal Physiol 299: F785-F791, 2010, showing binding of tamsulosin to the LC in Pons).

This class of molecules is of key importance in the formulation of compounds and pharmacologic treatment for WCS/FIRMR, due to their direct antagonistic effects on α1 adrenergic receptors located on noradrenergic neurons in the central nervous system (e.g. cortex, thalamus, brainstem, spinal cord) and vascular and muscle tissue (e.g. smooth and skeletal) in the periphery.

α-1 adrenergic receptor antagonists (AARAs) are used to inhibit FIMR in animal models, but have not been demonstrated to be effective in humans or animals for F/FA induced WCS/FIRMR. In various embodiments, selective or non-selective antagonists are used either singly or in combination to minimize the effects of AARA on blood pressure and will use delivery into the CNS via nasal insufflation to minimize the peripheral effects of AARAs on blood pressure. In addition, in certain embodiments AARAs are used in combination with vasoactive and cholinergic agents to offset, counteract, or minimize the effects of the unfavorable effects of AARAs on blood pressure and hemodynamics. This may be particularly helpful at times of overdose resuscitation, at which time most patients will be hemodynamically depressed. These combinations can be used in either immediate reversal or in prophylaxis embodiments.

TAMSULOSIN: Dose (0.4-0.8 mg QD); incidence of hypotension, syncope, vertigo is 0.2%-0.6% (˜1 in 500). Tamsulosin hydrochloride is a selective antagonist of α1A adrenoceptors in the prostate. Tamsulosin hydrochloride is (−)-(R)-5-[2-[[2-(o-Ethoxyphenoxy) ethyl]amino]propyl]-2-methoxybenzenesulfon-amide, monohydrochloride. Tamsulosin hydrochloride is a white crystalline powder that melts with decomposition at approximately 230° C. It is sparingly soluble in water and methanol, slightly soluble in glacial acetic acid and ethanol, and practically insoluble in ether.

The empirical formula of tamsulosin hydrochloride is C₂₀H₂₈N₂O₅S.HCl. The molecular weight of tamsulosin hydrochloride is 444.98. Its structural formula is:

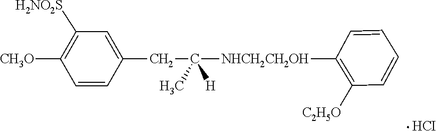

PRAZOSIN: Dose is 1 mg BID/TID and can be titrated up to 20 mg total QD in divided doses 5-6 mg TID). Syncope and symptoms of hypotension are 6-12% of subjects receiving (˜90 in 900).

MINIPRESS® (prazosin hydrochloride), a quinazoline derivative, is the first of a new chemical class of antihypertensives. It is the hydrochloride salt of 1-(4-amino-6,7-dimethoxy-2-quinazolinyl)-4-(2-furoyl) piperazine and its structural formula is:

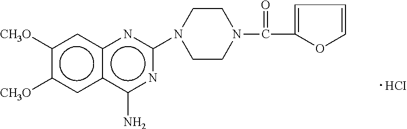

It is a white, crystalline substance, slightly soluble in water and isotonic saline, and has a molecular weight of 419.87. Each 1 mg capsule of MINIPRESS for oral use contains drug equivalent to 1 mg freebase. Molecular formula C₁₉H₂₁N₅O₄.HCl.

TERAZOSIN (dose 1-5 mg QD and NTE 20 mg QD) causes significant hypotension like prazosin with postural hypotension levels of 4% in trial of 600 subjects. syncope was 0.6%. HYTRIN (terazosin hydrochloride), an α-1-selective adrenoceptor blocking agent, is a quinazoline derivative represented by the following chemical name and structural formula: (RS)-Piperazine,1-(4-amino-6,7-dimethoxy-2-quinazolinyl)-4-[(tetra-hydro-2-furanyl)carbonyl]-, monohydrochloride, dihydrate.

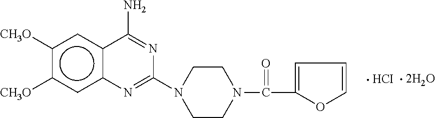

Terazosin hydrochloride is a white, crystalline substance, freely soluble in water and isotonic saline and has a molecular weight of 459.93. HYTRIN tablets (terazosin hydrochloride tablets) for oral ingestion are supplied in four dosage strengths containing terazosin hydrochloride equivalent to 1 mg, 2 mg, 5 mg, or 10 mg of terazosin.

SILODOSIN: (Dose: 8 mg QD) Study of 897 subjects with 3% with Dizziness and orthostatic hypotension and 1/897 with syncope.

RAPAFLO is the brand name for silodosin, a selective antagonist of α-1 adrenoreceptors. (3-Hydroxypropyl)-5-[(2R)-2-({2-[2-(2,2,2trifluoroethoxy)phenoxy]ethyl}amino)propyl]-2,3-dihydro-1H-indole-7-carboxamide and the molecular formula is C₂₅H₃₂F₃N₃O₄ with a molecular weight of 495.53. The structural formula of silodosin is:

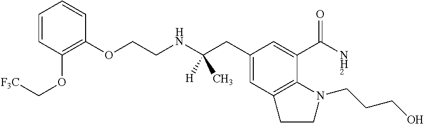 [SMILES: C[C@H](Cc1cc2c(c(C(N)=O)c1)N(CCCO)CCC2)NCCOc1ccccc1OCC(F)(F)F]

Silodosin is a white to pale yellowish white powder that melts at approximately 105 to 109° C. It is very soluble in acetic acid, freely soluble in alcohol, and very slightly soluble in water.

ALFUZOSIN: (Dose: 10-15 mg) 473 test subjects 6% had dizziness, 1/473 0.2% with syncope and 2/473 0.4% with hypotension. UROXATRAL® (alfuzosin HCl) Extended-release Tablets

Each UROXATRAL extended-release tablet contains 10 mg alfuzosin hydrochloride as the active ingredient. Alfuzosin hydrochloride is a white to off-white crystalline powder that melts at approximately 240° C. It is freely soluble in water, sparingly soluble in alcohol, and practically insoluble in dichloromethane. Alfuzosin hydrochloride is (R,S)—N-[3-[(4-amino-6,7-dimethoxy-2-quinazolinyl) methylamino] propyl] tetrahydro-2-furancarboxamide hydrochloride. The empirical formula of alfuzosin hydrochloride is C₁₉H₂₇N₅O₄.HCl. The molecular weight of alfuzosin hydrochloride is 425.9. Its structural formula is:

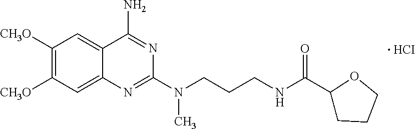

DOXAZOSIN: (dose: 1 mg QD NTE 16 mg, dose may be titrated up to 2 mg q 1-2 weeks; 1-16 mg in HTN and 0.5-8 mg in normotensives) 965 test subjects Dizzy 15-19% and Hypotension in 1.7%.

CARDURA® (doxazosin mesylate) CARDURA® (doxazosin mesylate) is a quinazoline compound that is a selective inhibitor of the α1 subtype of α-adrenergic receptors. The chemical name of doxazosin mesylate is 1-(4-amino-6,7-dimethoxy-2-quinazolinyl)-4-(1,4benzodioxan-2-ylcarbonyl) piperazine methanesulfonate. The empirical formula for doxazosin mesylate is C₂₃H₂₅N₅O₅.CH₄O₃S and the molecular weight is 547.6. It has the following structure:

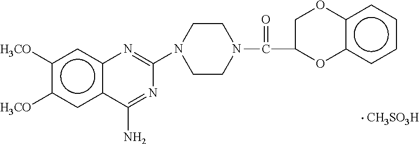

CARDURA (doxazosin mesylate) is freely soluble in dimethylsulfoxide, soluble in dimethylformamide, slightly soluble in methanol, ethanol, and water (0.8% at 25° C.), and very slightly soluble in acetone and methylene chloride. CARDURA is available as colored tablets for oral use and contains 1 mg (white), 2 mg (yellow), 4 mg (orange) and 8 mg (green) of doxazosin as the free base.

(b) Mu and/or Opioid Receptor Subtype Antagonists

Mu receptor antagonists are used for alleviating or inhibiting the dose dependent respiratory depression caused all opiates/opioids and can vary in their effects at opioid receptor subtypes (delta, kappa, mu). Short duration and rapid acting agents (e.g. naloxone, narcan) are used for immediate reversal, while longer acting agents (e.g. naltrexone) are used for prophylaxis. MU receptor antagonists include Naloxone, Naltrexone, Nalmefene, nalorphine, and Levallorphan.

NALOXONE—NARCAN® (dose 0.4-2 mg IV and may repeat dose up to 10 mg. May also be dosed IM, SC, intranasal) (naloxone hydrochloride) NARCAN (naloxone hydrochloride injection, USP), an opioid antagonist, is a synthetic congener of oxymorphone. In structure it differs from oxymorphone in that the methyl group on the nitrogen atom is replaced by an allyl group; the structure is provided below.

Naloxone hydrochloride occurs as a white to slightly off-white powder, and is soluble in water, in dilute acids, and in strong alkali; slightly soluble in alcohol; practically insoluble in ether and in chloroform. NARCAN (naloxone) injection is available as a sterile solution for intravenous, intramuscular and subcutaneous administration in three concentrations: 0.02 mg, 0.4 mg and 1 mg of naloxone hydrochloride per mL. pH is adjusted to 3.5±0.5 with hydrochloric acid. The 0.02 mg/mL strength is an unpreserved, paraben-free formulation containing 9 mg/mL sodium chloride.

NARCAN (naloxone) may be diluted for intravenous infusion in normal saline or 5% dextrose solutions. Naloxone is indicated for the complete or partial reversal of opioid depression, including respiratory depression, induced by natural and synthetic opioids. NARCAN (naloxone) is also indicated for diagnosis of suspected or known acute opioid overdosage. If an opioid overdose—is known or suspected: an adult initial dose of 0.4 mg to 2 mg of NARCAN (naloxone) may be administered intravenously, IM, subcutaneously or nasally. If the desired degree of counteraction and improvement in respiratory functions are not obtained, it may be repeated at two- to three-minute intervals. If no response is observed after 10 mg of NARCAN (naloxone) have been administered, the diagnosis of opioid-induced or partial opioid-induced toxicity should be questioned. If necessary, NARCAN (naloxone) can be diluted with sterile water for injection.

NALOXONE NASAL SPRAY FORMULATION: NARCAN® (naloxone hydrochloride) Nasal Spray. NARCAN (naloxone hydrochloride) Nasal Spray is a pre-filled, single dose intranasal spray. Chemically, naloxone hydrochloride is the hydrochloride salt of 17-Allyl-4,5α-epoxy-3,14-dihydroxymorphinan-6-one hydrochloride with the following structure:

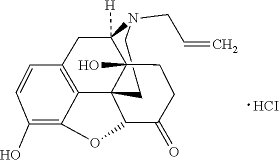

Naloxone hydrochloride, an opioid antagonist, occurs as a white to slightly off-white powder, and is soluble in water, in dilute acids, and in strong alkali; slightly soluble in alcohol; practically insoluble in ether and in chloroform. Each NARCAN Nasal Spray contains a single 4 mg dose of naloxone hydrochloride in a 0.1 MI intranasal spray. Inactive ingredients include benzalkonium chloride (preservative), disodium ethylenediaminetetraacetate (stabilizer), sodium chloride, hydrochloric acid to adjust pH, and purified water. The pH range is 3.5 to 5.5. NARCAN Nasal Spray is indicated for the emergency treatment of known or suspected opioid overdose, as manifested by respiratory and/or central nervous system depression. NARCAN Nasal Spray is intended for immediate administration as emergency therapy in settings where opioids may be present.

NALTREXONE: REVIA® (DOSE 25-50 MG PO QD) (naltrexone hydrochloride) Tablets USP 50 mg-long acting opioid antagonist. REVIA® (naltrexone hydrochloride tablets USP), an opioid antagonist, is a synthetic congener of oxymorphone with no opioid agonist properties. Naltrexone differs in structure from oxymorphone in that the methyl group on the nitrogen atom is replaced by a cyclopropylmethyl group. REVIA is also related to the potent opioid antagonist, naloxone, or n-allylnoroxymorphone.

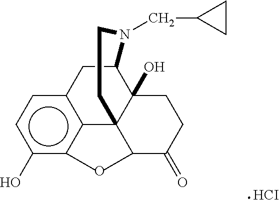

REVIA is a white, crystalline compound. The hydrochloride salt is soluble in water to the extent of about 100 mg/mL. REVIA is available in scored film-coated tablets containing 50 mg of naltrexone hydrochloride. REVIA Tablets also contain: colloidal silicon dioxide, crospovidone, hydroxypropyl methylcellulose, lactose monohydrate, magnesium stearate, microcrystalline cellulose, polyethylene glycol, polysorbate 80, synthetic red iron oxide, synthetic yellow iron oxide and titanium dioxide.

VIVITROL®—NALTREXONE INJECTABLE: Extended-release Injectable Suspension: VIVITROL® (naltrexone for extended-release injectable suspension) is supplied as a microsphere formulation of naltrexone for suspension, to be administered by intramuscular injection. Naltrexone is an opioid antagonist with little, if any, opioid agonist activity. Naltrexone is designated chemically as morphinan-6-one, 17 (cyclopropylmethyl) 4,5-epoxy3,14-dihydroxy-(5a) (CAS Registry #16590-41-3). The molecular formula is C₂₀H₂₃NO₄ and its molecular weight is 341.41 in the anhydrous form (i.e., <1% maximum water content). The structural formula is:

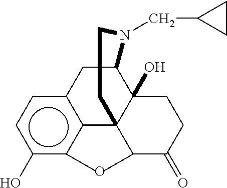

Naltrexone base anhydrous is an off-white to a light tan powder with a melting point of 168-170° C. (334-338° F.). It is insoluble in water and is soluble in ethanol. VIVITROL is commercially available as a carton containing a vial each of VIVITROL microspheres and diluent, one 5-mL syringe, one 1-inch 20-gauge preparation needle, two 1°-inch 20-gauge and two 2-inch 20-gauge administration needles with needle protection device. VIVITROL microspheres consist of a sterile, off-white to light tan powder that is available in a dosage strength of 380 mg of naltrexone per vial. Naltrexone is incorporated in 75:25 polylactide-co-glycolide (PLG) at a concentration of 337 mg of naltrexone per gram of microspheres. The diluent is a clear, colorless solution. The composition of the diluent includes carboxymethylcellulose sodium salt, polysorbate 20, sodium chloride, and water for injection. The microspheres must be suspended in the diluent prior to injection.

NALMEFENE: REVEX (nalmefene hydrochloride) Injection, Solution.

REVEX (nalmefene hydrochloride injection), an opioid antagonist, is a 6-methylene analogue of naltrexone. The chemical structure is shown below:

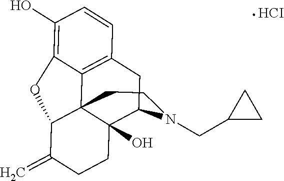

Molecular Formula: C₂₁H₂₅NO₃.HCl; Molecular Weight: 375.9, CAS #58895-64-0; Chemical Name: 17-(Cyclopropylmethyl)-4,5α-epoxy-6-methylenemorphinan-3,14-diol, hydrochloride salt.

Nalmefene hydrochloride is a white to off-white crystalline powder which is freely soluble in water up to 130 mg/mL and slightly soluble in chloroform up to 0.13 mg/mL, with a pKa of 7.6.

REVEX is available as a sterile solution for intravenous, intramuscular, and subcutaneous administration in two concentrations, containing 100 μg or 1.0 mg of nalmefene free base per mL. The 100 μg/mL concentration contains 110.8 μg of nalmefene hydrochloride and the 1.0 mg/mL concentration contains 1.108 mg of nalmefene hydrochloride per mL. Both concentrations contain 9.0 mg of sodium chloride per mL and the pH is adjusted to 3.9 with hydrochloric acid. Concentrations and dosages of REVEX are expressed as the free base equivalent of nalmefene.

REVEX is indicated for the complete or partial reversal of opioid drug effects, including respiratory depression, induced by either natural or synthetic opioids. REVEX is indicated in the management of known or suspected opioid overdose. REVEX should be titrated to reverse the undesired effects of opioids. Once adequate reversal has been established, additional administration is not required and may actually be harmful due to unwanted reversal of analgesia or precipitated withdrawal.

### (c) Vasopressors

Vasoactive agents are used to offset, counteract, or minimize the effects of the unfavorable effects of AARAs on blood pressure and hemodynamics.

Ephedrine: Ephedrine Sulfate-: 5-10 MG (standard concentration at 10 mg) may repeat Q 2-3″ IV, IM, IN Injection, USP; For IM, IV or SC Use 50 mg/mL pH 4.5-7.0

Ephedrine Sulfate Injection, USP is a sterile solution of 50 mg ephedrine sulfate in water for injection. Ephedrine occurs as fine, white, odorless crystals or powder and darkens on exposure to light. It is freely soluble in water and sparingly soluble in alcohol. The chemical name for ephedrine sulfate is (C₁₀H₁₅NO)₂.H₂SO₄ benzenemethanol α-[1-(methylamino) ethyl]-sulfate (2:1) (salt). Its molecular weight is 428.54. The structural formula is:

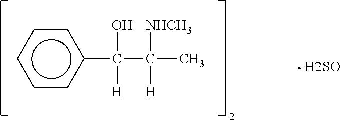

The drug has long been used as a pressor agent, particularly during spinal anesthesia when hypotension frequently occurs. It is indicated as a central nervous system stimulant in narcolepsy and depressive states. The usual parenteral dose is 25 to 50 mg given subcutaneously or intramuscularly. Intravenously, 5 to 25 mg may be administered slowly, repeated in 5 to 10 minutes, if necessary. The usual subcutaneous or intramuscular dose is 0.5 mg/kg of body weight or 16.7 mg/square meter of body surface every 4 to 6 hours. Parenteral drug products should be inspected visually for particulate matter and discoloration prior to administration, whenever solution and container permit. Commercially available as: Ephedrine Sulfate Injection, USP, 50 mg/mL, 1 mL per vial, NDC 55390-875-0. Packed 25 vials per carton. Protect from light. Manufactured for: Bedford Laboratories™ Bedford, Ohio 44146. Manufactured by: Ben Venue Laboratories Inc. Bedford, Ohio 44146.

Phenylephrine: VAZCULEP® PHENYLEPHRINE: 10-100 MCG (standard concentration at 50 mcg) may repeat Q 2-3″ (25-200 mcg/min IV) PHENYLEPHRINE: may be bolused 10-200 mcg IV or may be given via IV infusion 20 mg of Phenylephrine in 250 ml of 5% dextrose in distilled water (80 mcg/ml) IV infusion rate NTE (25-200 mcg/min). Phenylephrine is an alpha-1 adrenergic receptor agonist. VAZCULEP (phenylephrine hydrochloride) Injection, 10 mg/mL, is a sterile, nonpyrogenic solution for intravenous use. It must be diluted before administration as an intravenous bolus or continuous intravenous infusion. The chemical name of phenylephrine hydrochloride is (−)-m-hydroxy-a-[(methylamino)methyl]benzyl alcohol hydrochloride, and its structural formula is depicted below:

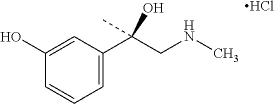

Phenylephrine hydrochloride is soluble in water and ethanol, and insoluble in chloroform and ethyl ether. VAZCULEP (phenylephrine hydrochloride) Injection, 10 mg/mL, is sensitive to light. Each mL contains: phenylephrine hydrochloride 10 mg, sodium chloride 3.5 mg, sodium citrate dihydrate 4 mg, citric acid monohydrate 1 mg, and sodium metabisulfite 2 mg in water for injection. The pH is adjusted with sodium hydroxide and/or hydrochloric acid if necessary. The pH range is 3.5-5.5. VAZCULEP (phenylephrine hydrochloride) Injection, 10 mg/mL, is an alpha-1 adrenergic receptor agonist indicated for the treatment of clinically important hypotension resulting primarily from vasodilation in the setting of anesthesia. VAZCULEP (phenylephrine hydrochloride) The following are the recommended dosages for the treatment of hypotension. The recommended initial dose is 40 to 100 mcg administered by intravenous bolus. Additional boluses may be administered every 1-2 minutes as needed; not to exceed a total dosage of 200 mcg.

Epinephrine: (epinephrine) Injection— EPINEPHRINE is to be used with caution in individuals with F/FAs overdose due to the direct and potent activity of Epinephrine and Noradrenaline at the LC and FIMR/WCS/FIRMR related circuitry. However, should this be the initial presentation in suspected opioid overdose, the medical practitioner should use their discretion to follow best practices and go directly to the most current ACLS treatment algorithms with the possible addition of the baseline formulation for WCS/FIRMR reversal. The ACLS dose protocol for CARDIAC ARREST is: 1 mg IV and may repeat Q2-3″ for total dose of 3 mg or Infusion 1 mg EPINEPHRINE in 250 ml of D5W (4 mcg/ml) IV infusion rate NTE (1-4 mcg/min).

Epinephrine is a sympathomimetic catecholamine (a non-selective alpha and beta-adrenergic receptor agonist) designated chemically as (−)-3,4-Dihydroxy-α-[(methylamino) methyl]benzyl alcohol, has a molecular weight of 183.20, and has the following structure:

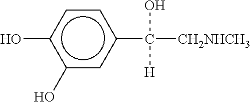

### (d) Anticholinergics

In certain embodiments, anticholinergic agents can be used herein, in patients who are either bradycardic, asystolic, to decrease vagal tone (baseline heart rate) or to alleviate cholinergically mediated closure of vocal cords/laryngospasm in patients who are using these drugs for prophylaxis or immediate reversal of F/FA overdose or toxic exposure. To alleviate cholinergically mediated closure of vocal cords/laryngospasm, an anticholinergic agent (e.g. atropine) can be given in a fully vagolytic dose (10-50 mcg/kg) for the dual effect of preventing bradycardia and to modify possible fentanyl M3 antagonist effects on vagal motor nuclei controlling laryngeal muscle patency where either a cholinergic agent (muscarinic receptor antagonist/anticholinergic, M3 receptor agonist or a general nicotinic receptor agonist or selective agonist) can be used to reverse laryngospasm or restore laryngeal muscle patency.

ATROPINE: (dose 0.5-2 mg IV and can be given IM, SC, intranasally and via endotracheal tube and possibly intraocular with eye drops. Atropine, an anticholinergic agent (muscarinic antagonist), occurs as white crystals, usually needle-like, or as a white, crystalline powder. It is highly soluble in water with a molecular weight of 289.38. Atropine, a naturally occurring belladonna alkaloid, is a racemic mixture of equal parts of d-and l-hyoscyamine; its activity is due almost entirely to the levo isomer of the drug. Chemically, atropine is designated as 1H,5H-Tropan-3-ol (±)-tropate. Its empirical formula is C₁₇H₂₃NO₃ and its structural formula is:

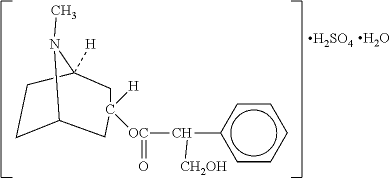

Atropine Sulfate Injections, USP, are indicated when excessive or sometime normal) muscarinic effects are judged to be life threatening or are producing symptoms severe enough to require reversible muscarinic blockade. Examples, not an exhaustive list, of such possible uses are: to decrease vagal tone (baseline heart rate) or to alleviate cholinergically mediated closure of vocal cords/laryngospasm in patients who are using these drugs for prophylaxis or immediate reversal of F/FA overdose or toxic exposure. Atropine Sulfate Injection, USP in A Syringe is intended for intravenous use, but may be administered subcutaneously or intramuscularly. Its use usually requires titration, using heart rate, PR interval, blood pressure and/or patient's symptoms as a guide for having reached an appropriate dose. Initial single doses in adults vary from around 0.5 mg to 1 mg (5-10 mL of the 0.1 mg/mL solution) for antisialagogue and other antivagal effects, to 2 to 3 mg (20-30 mL of the 0.1 mg/mL solution). When used as an antidote, the 2 to 3 mg dose should be repeated no less often that every 20 to 30 minutes until signs of poisoning are sufficiently lessened or signs of atropine poisoning occur. When the recurrent use of atropine is essential in patients with coronary artery disease, the total dose should be restricted to 2 to 3 mg (maximum 0.03 to 0.04 mg/kg) to avoid the detrimental effects of atropine-induced tachycardia on myocardial oxygen demand. Three milligrams (0.04 mg/kg) given I.V. is a fully vagolytic dose in most patients. The administration of less than 0.5 mg can produce a paradoxical bradycardia because of the central or peripheral para-sympathomimetic effects of low dose in adults. In the case of F/FA overdose or toxic exposure, either the use of a fully vagolytic dose of a muscarinic anticholinergic (e.g. to antagonize M1-M5 receptors) or the use of a cholinergic agonist at M3 receptors (e.g. pilocarpine) to alleviate cholinergically mediated closure of vocal cords in patients who are using these drugs for prophylaxis or immediate reversal of F/FA overdose or toxic exposure and to modify possible fentanyl M3 antagonist effects on vagal motor nuclei controlling laryngeal muscle patency). Endotracheal administration of atropine can be used in patients without I.V. access. The recommended adult dose of atropine for endotracheal administration is 1 to 2 mg diluted to a total not to exceed 10 ml of sterile water or normal saline.

Glycopyrrolate “ROBINUL” ™ (Dose: 0.1-1 mg IV) ROBINUL (glycopyrrolate) Injection is a synthetic anticholinergic agent. Each 1 mL contains: Glycopyrrolate, USP 0.2 mg, water for Injection, USP q.s., Benzyl Alcohol, NF 0.9% (preservative); pH adjusted, when necessary, with hydrochloric acid and/or sodium hydroxide. Formulated for Intramuscular (IM) or Intravenous (IV) administration. Glycopyrrolate is a quaternary ammonium salt with the following chemical name: 3[(cyclopentylhydroxyphenylacetyl)oxy]-1,1-dimethyl pyrrolidinium bromide. The molecular formulas is C₁₉H₂₈BrNO₃ and the molecular weight is 398.33. Its structural formula is as follows:

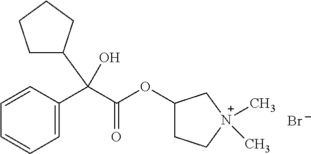 [SMILES: C[N+]1(C)CCC(OC(=O)C(O)(c2ccccc2)C2CCCC2)C1 ; [Br-]]

Glycopyrrolate occurs as a white, odorless crystalline powder. It is soluble in water and alcohol, and practically insoluble in chloroform and ether. Unlike atropine, glycopyrrolate is completely ionized at physiological pH values. ROBINUL (glycopyrrolate) Injection is a clear, colorless, sterile liquid; pH 2.0-3.0. The partition coefficient of glycopyrrolate in a n-octanol/water system is 0.304 (log₁₀ P=−1.52) at ambient room temperature (24° C.). ROBINUL Injection is indicated for use as a preoperative antimuscarinic to reduce salivary, tracheobronchial, and pharyngeal secretions; to reduce the volume and free acidity of gastric secretions; and to block cardiac vagal inhibitory reflexes during induction of anesthesia and intubation. When indicated, ROBINUL Injection may be used intraoperatively to counteract surgically or drug induced or vagal reflexes associated arrhythmias. Glycopyrrolate protects against the peripheral muscarinic effects (e.g., bradycardia and excessive secretions) of cholinergic agents such as neostigmine and pyridostigmine given to reverse the neuromuscular blockade due to nondepolarizing muscle relaxants. In the case of F/FA overdose or toxic exposure, either the use of a fully vagolytic dose of a muscarinic anticholinergic (antagonize M1-M5 receptors) or the use of a cholinergic agonist at M3 receptors (e.g., pilocarpine) in some cases may be used to alleviate cholinergically mediated closure of vocal cords in patients who are using these drugs for prophylaxis or immediate reversal of F/FA overdose or toxic exposure and to modify possible fentanyl M3 antagonist effects on vagal motor nuclei controlling laryngeal muscle patency.

The recommended adult dose of ROBINUL Injection that may be used to counteract drug-induced or vagal reflexes and their associated arrhythmias (e.g., bradycardia) should be administered intravenously as single doses of 0.1 mg and repeated, as needed, at intervals of 2 to 3 minutes.

Droperidol: Molecular Formula: C₂₂H₂₂FN₃O₂; represented by the following structural formula:

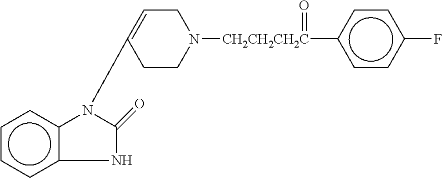

Droperidol is a butyrophenone with anti-emetic, sedative and anti-anxiety properties. Droperidol is a neuroleptic (tranquilizer) agent chemically designated as 1-[1-[3-(p-Fluorobenzoyl) propyl]-1,2,3,6-tetrahydro-4-pyridyl]-2-benzimidazolinone with a molecular weight of 379.43. Droperidol may block dopamine receptors in the chemoreceptor trigger zone (CTZ), which may lead to its anti-emetic effect. This agent may also bind to postsynaptic gamma-aminobutyric acid (GABA) receptors in the central nervous system (CNS), which increases the inhibitory effect of GABA and leads to sedative and anti-anxiety activities.

Droperidol produces mild alpha-adrenergic blockade, peripheral vascular dilatation and reduction of the pressor effect of epinephrine. It can produce hypotension and decreased peripheral vascular resistance and may decrease pulmonary arterial pressure (particularly if it is abnormally high). It may reduce the incidence of epinephrine-induced arrhythmias but it does not prevent other cardiac arrhythmias. The onset of action of single intramuscular and intravenous doses is from three to ten minutes following administration, although the peak effect may not be apparent for up to thirty minutes. The duration of the tranquilizing and sedative effects generally is two to four hours, although alteration of alertness may persist for as long as twelve hours.

Droperidol dosage should be individualized. Some of the factors to be considered in determining dose are age, body weight, physical status, underlying pathological condition, use of other drugs, the type of anesthesia to be used, and the surgical procedure involved. Vital signs and ECG should be monitored routinely. Adult Dosage: The maximum recommended initial dose of Droperidol is 2.5 mg I.M. or slow I.V. Additional 1.25 mg doses of Droperidol may be administered to achieve the desired effect. However, additional doses should be administered with caution, and only if the potential benefit outweighs the potential risk

PILOCARPINE: Molecular Formula: C₁₁H₁₆N₂O₂: represented by the following structural formula:

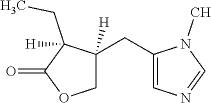

Pilocarpine is a choline ester miotic and a positively charged quaternary ammonium compound. Pilocarpine is a natural alkaloid extracted from plants of the genus Pilocarpus with cholinergic agonist activity. As a cholinergic parasympathomimetic agent, pilocarpine predominantly binds to muscarinic receptors, thereby inducing exocrine gland secretion and stimulating smooth muscle in the bronchi, urinary tract, biliary tract, and intestinal tract. Pilocarpine is used as its hydrochloride and possesses excitatory activity on the parasympathetic nerve system, like physostigmine and arecoline. Thus, this alkaloid acts as an antagonist of atropine and it promotes the secretion of sweat, saliva, and tears and causes myosis. It is reported that subcutaneous injection of 10 mg of pilocarpine HCl causes violent sweating (0.5-1.0 l) and salivation (1 l). A 1% solution of pilocarpine HCl can be used for IOC. When applied topically to the eye as a single dose it causes miosis, spasm of accommodation, and may cause a transitory rise in intraocular pressure followed by a more persistent fall.

Pilocarpine may have paradoxical effects on the cardiovascular system. The expected effect of a muscarinic agonist is vasodepression, but administration of pilocarpine may produce hypertension after a brief episode of hypotension. Bradycardia and tachycardia have both been reported with use of pilocarpine. Pilocarpine dosing information: representative Adult Dose: 5 mg three times a day. Titrate upwards, not to exceed 10 mg per dose, to a maximum of 30 mg per day.

### (e) Paralytics/Muscle Relaxants

Muscle relaxants and paralytics such as succinylcholine and rocuronium are rapid acting and are used to alleviate WCS/FIRMR particularly in the chest wall and diaphragm, and may relieve spasm of the vocal cords and larynx. Low doses (on the order 1-3 mg for Succinylcholine) can be used to decrease FIMR without compromise of AW reflexes. These drugs are generally used in immediate resuscitation scenarios by individuals who are trained in invasive AW management and are used at full intubation doses (0.5-1.1 mg/kg).

Succinylcholine: (Anectine, QUELICIN™) For reversal or inhibition of fentanyl or fentanyl analogue induced muscle rigidity—FIMR in an adult patient, dose is 0.01-0.05 mg/kg. However, to fully secure the airway with endotracheal intubation, the dose is 0.3-1.1 mg/kg. QUELICIN™ (succinylcholine chloride) Injection, USP

QUELICIN (Succinylcholine Chloride Injection, USP) is a sterile, nonpyrogenic solution to be used as a short-acting, depolarizing, skeletal muscle relaxant. The solutions are for I.M. or I.V. use. Succinylcholine Chloride, USP is chemically designated C₁₄H₃₀Cl₂N₂O and its molecular weight is 361.31. It has the following structural formula:

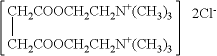

Succinylcholine is a diquaternary base consisting of the dichloride salt of the dicholine ester of succinic acid. It is a white, odorless, slightly bitter powder, very soluble in water. The drug is incompatible with alkaline solutions but relatively stable in acid solutions. Solutions of the drug lose potency unless refrigerated. Solution intended for multiple-dose administration contains 0.18% methylparaben and 0.02% propylparaben as preservatives (List No. 6629). Solution intended for single-dose administration contains no preservatives. May contain sodium hydroxide and/or hydrochloric acid for pH adjustment. pH is 3.6 (3.0 to 4.5). Succinylcholine chloride is indicated as an adjunct to general anesthesia, to facilitate tracheal intubation, and to provide skeletal muscle relaxation during surgery or mechanical ventilation and for the treatment of fentanyl induced chest wall or muscle rigidity (Janssen Pharmaceuticals package insert for “Sublimaze-Fentanyl”).

The dosage of succinylcholine should be individualized and should always be determined by the clinician after careful assessment of the patient. For Reversal or inhibition of fentanyl or fentanyl analogue induced rigidity in an adult patient, dose is 0.01-0.05 mg/kg. However, for full scale securing of the airway with endotracheal intubation in severe WCS, the dose is 0.3-1.1 mg/kg. Following administration of doses in this range, neuromuscular blockade develops in about 1 minute; maximum blockade may persist for about 2 minutes, after which recovery takes place within 4 to 6 minutes. However, very large doses may result in more prolonged blockade. A 5 to 10 mg test dose may be used to determine the sensitivity of the patient and the individual recovery time.

Whereas bradycardia is common in pediatric patients after an initial dose of 1.5 mg/kg, bradycardia is seen in adults only after repeated exposure. The occurrence of bradyarrhythmias may be reduced by pretreatment with atropine.

If necessary, succinylcholine may be given intramuscularly to adults when a suitable vein is inaccessible. A dose of up to 3 to 4 mg/kg may be given, but not more than 150 mg total dose should be administered by this route. The onset of effect of succinylcholine given intramuscularly is usually observed in about 2 to 3 minutes.

Succinylcholine is acidic (pH 3.5) and should not be mixed with alkaline solutions having a pH greater than 8.5 (e.g., barbiturate solutions). QUELICIN™ (Succinylcholine Chloride Injection, USP) is supplied as a clear, colorless solution. Refrigeration of the undiluted agent will assure full potency until expiration date. All units carry a date of expiration. Store in refrigerator 2° to 8° C. (36° to 46° F.). The multi-dose vials are stable for up to 14 days at room temperature without significant loss of potency.

ROCURONIUM: ZEMURON® (rocuronium bromide) Injection (dose: for intubation 0.4-1.2 mg/kg and for treatment of FIMR 0.005-0.01 mg/kg). ZEMURON (rocuronium bromide) injection is a nondepolarizing neuromuscular blocking agent with a rapid to intermediate onset depending on dose and intermediate duration. Rocuronium bromide is chemically designated as 1-[17β-(acetyloxy)-3α-hydroxy-2β-(4-morpholinyl)-5α-androstan-16β-yl]-1-(2-propenyl)pyrrolidinium bromide. The structural formula is:

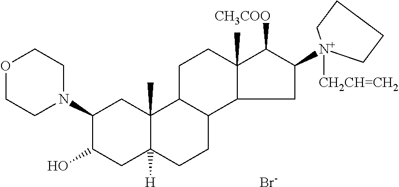

The chemical formula is C₃₂H₅₃BrN₂O₄ with a molecular weight of 609.70. The partition coefficient of rocuronium bromide in n-octanol/water is 0.5 at 20° C. ZEMURON is supplied as a sterile, nonpyrogenic, isotonic solution that is clear, colorless to yellow/orange, for intravenous injection only. Each mL contains 10 mg rocuronium bromide and 2 mg sodium acetate. The aqueous solution is adjusted to isotonicity with sodium chloride and to a pH of 4 with acetic acid and/or sodium hydroxide. ZEMURON® (rocuronium bromide) Injection is indicated for inpatients and outpatients as an adjunct to general anesthesia to facilitate both rapid sequence and routine tracheal intubation, and to provide skeletal muscle relaxation during surgery or mechanical ventilation and for the treatment of fentanyl induced muscle rigidity—FIMR or WCS.

ZEMURON is for intravenous use only. This drug should only be administered by experienced clinicians or trained individuals supervised by an experienced clinician familiar with the use, actions, characteristics, and complications of neuromuscular blocking agents. Doses of ZEMURON injection should be individualized and a peripheral nerve stimulator should be used to monitor drug effect, need for additional doses, adequacy of spontaneous recovery or antagonism, and to decrease the complications of overdosage if additional doses are administered. The dosage information which follows is derived from studies based upon units of drug per unit of body weight. It is intended to serve as an initial guide to clinicians familiar with other neuromuscular blocking agents to acquire experience with ZEMURON. The recommended initial dose of ZEMURON, regardless of anesthetic technique, is 0.6 mg/kg. Neuromuscular block sufficient for intubation (80% block or greater) is attained in a median (range) time of 1 (0.4-6) minute(s) and most patients have intubation completed within 2 minutes. Maximum blockade is achieved in most patients in less than 3 minutes. In appropriately premedicated and adequately anesthetized patients, ZEMURON 0.6 to 1.2 mg/kg will provide excellent or good intubating conditions in most patients in less than 2 minutes.

### (f) Centrally Acting Respiratory Stimulants

Respiratory accelerants (stimulants) can be used in an immediate resuscitation scenario to synergistically impact and reverse the inhibitory effects of opiates/opioids on the CO₂ chemoreceptors located in the carotid body, aortic body, and possibly the heart. Opiates depress respiratory drive by depressing the reactivity and response of these CO₂ chemo-sensors to increase respiratory drive in the face of increasing serum levels of CO₂. This is inhibition of hypoxia driven respiratory drive is a significant way that opiates cause hypoxemia in opioid overdose. Other cholinergic agonists (e.g. nicotine) or antagonists with activity on central respiratory neurons (e.g. pontine Kölliker-Fuse neurons) can also be used in combination with these respiratory accelerants. To be used in immediate resuscitation scenarios.

One representative nicotinic receptor agonist is nicotine. The chemical formula of nicotine is C₁₀H₁₄N₂; which is represented by the following structural formula:

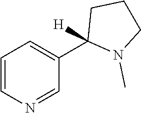

Nicotine is a hygroscopic, colorless to yellow-brown, oily liquid, that is readily soluble in alcohol, ether or light petroleum. It is miscible with water in its base form between 60° C. and 210° C. As a nitrogenous base, nicotine forms salts with acids that are usually solid and water-soluble. Its flash point is 95° C. and its auto-ignition temperature is 244° C. Nicotine is readily volatile (vapor pressure 5.5 Pa at 25° C.) and dibasic (Kb₁=1×10⁻⁶, Kb₂=1×10⁻¹¹). Nicotine is a stimulant and potent parasympathomimetic alkaloid that is naturally produced in the nightshade family of plants. It is used for the treatment of tobacco use disorders as a smoking cessation aid and nicotine dependence for the relief of withdrawal symptoms. Nicotine acts as a receptor agonist at most nicotinic acetylcholine receptors (nAChRs), except at two nicotinic receptor subunits (nAChRα9 and nAChRα10) where it acts as a receptor antagonist. By binding to nicotinic acetylcholine receptors in the brain, nicotine elicits its psychoactive effects and increases the levels of several neurotransmitters in various brain structures—acting as a sort of “volume control. Nicotine has a higher affinity for nicotinic receptors in the brain than those in skeletal muscle, though at toxic doses it can induce contractions and respiratory paralysis. As nicotine enters the body, it is distributed quickly through the bloodstream and crosses the blood-brain barrier reaching the brain within 10-20 seconds after inhalation. The elimination half-life of nicotine in the body is around two hours. Nicotine is primarily excreted in urine and urinary concentrations vary depending upon urine flow rate and urine pH. Nicotine has a half-life of ˜1-2 hours. Nicotine has potential interaction with sympathomimetic drugs (adrenergic agonists) and sympatholytic drugs (alpha-blockers and beta-blockers).

### (g) Anti-Seizure/Membrane-Stabilizing Agents

Another aspect provided herein deals with overdose due to a combination of one or more opiates/opioids and at least one benzodiazepine sedative-hypnotic agent. Because benzodiazepine use (particularly when used chronically, and/or with alcohol) causes an upregulation of adrenergic receptors in the brain, abrupt reversal of drug overdoses where benzodiazepine use is suspected must be carefully monitored to avoid or minimize the risk of seizures precipitated by rapid reversal. Thus, in some embodiments, the combination therapy includes an anti-seizure medication/membrane stabling agent, such as Dilantin as prophylaxis for seizure activity.

DILANTIN® (phenytoin sodium) Injection, for Intravenous or Intramuscular Use

Dilantin: ASMS: DILANTIN:50 MG may repeat Q 2-3″ (NTE 50 mg/min rapid IV infusion) IV, IN. In the event of “status epilepticus” induced by rapid reversal of benzodiazepine overdose, a conversion to use of separate baseline reversal drug (e.g. MU+NS-A1ARA+S-A1ARA+/−AC or C) with IV Dilantin (5-15 mg/kg) with infusion rate NTE 50 mg/min due to risk of cardiac arrhythmia.

The rate of intravenous DILANTIN administration should not exceed 50 mg per minute in adults and 1 to 3 mg/kg/min (or 50 mg per minute, whichever is slower) in pediatric patients because of the risk of severe hypotension and cardiac arrhythmias. Careful cardiac monitoring is needed during and after administering intravenous DILANTIN. Although the risk of cardiovascular toxicity increases with infusion rates above the recommended infusion rate, these events have also been reported at or below the recommended infusion rate. Reduction in rate of administration or discontinuation of dosing may be needed.

DILANTIN (phenytoin sodium) injection, USP is a sterile solution of 50 mg phenytoin sodium per milliliter for intravenous or intramuscular administration. The solution is in a vehicle containing 40% propylene glycol and 10% alcohol in water for injection, adjusted to pH 12 with sodium hydroxide. Phenytoin sodium is related to the barbiturates in chemical structure, but has a five-membered ring. The chemical name is sodium 5,5-diphenyl-2, 4-imidazolidinedione represented by the following structural formula:

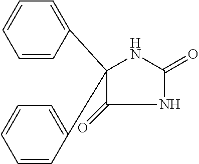 [SMILES: O=C1NC(=O)C(c2ccccc2)(c2ccccc2)N1]

### (h) α2-Adrenergic Receptor Agonists

In certain embodiments, alpha 2 agonists may be used in the inhibition or partial inhibition of fentanyl induced muscle rigidity. Optionally, these can be used with an al antagonist in various treatment methods. Clonidine is a representative α2-adrenergic receptor agonist.

Clonidine-CATAPRES® (clonidine hydrochloride) Oral Antihypertensive Tabs of 0.1, 0.2 and 0.3 mg, CATAPRES® (clonidine hydrochloride, USP) is a commercially available centrally acting alpha-agonist hypotensive agent available as tablets for oral administration in three dosage strengths: 0.1 mg, 0.2 mg and 0.3 mg. The 0.1 mg tablet is equivalent to 0.087 mg of the free base. The inactive ingredients are colloidal silicon dioxide, corn starch, dibasic calcium phosphate, FD&C Yellow No. 6, gelatin, glycerin, lactose, and magnesium stearate. Clonidine hydrochloride is an imidazoline derivative and exists as a mesomeric compound. The chemical name is 2-(2,6-dichlorophenylamino)-2-imidazoline hydrochloride; C₉H₉Cl₂N₃.HCl, Mol. Wt. 266.56. Clonidine hydrochloride is an odorless, bitter, white, crystalline substance soluble in water and alcohol. The following is the structural formula:

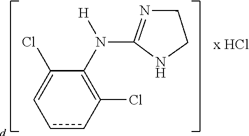

The following is a general guide to its administration. Initial dose: 0.1 mg tablet twice daily (morning and bedtime). Elderly patients may benefit from a lower initial dose. Maintenance Dose: Further increments of 0.1 mg per day may be made at weekly intervals if necessary until the desired response is achieved. Taking the larger portion of the oral daily dose at bedtime may minimize transient adjustment effects of dry mouth and drowsiness. The therapeutic doses most commonly employed have ranged from 0.2 mg to 0.6 mg per day given in divided doses. Studies have indicated that 2.4 mg is the maximum effective daily dose, but doses as high as this have rarely been employed. In the case of F/FA overdose or toxic exposure 0.05 mg-1 mg will be diluted into sterile water or NS for IV or IM injection in combination with other agents as noted in dosing charts.

### (i) GABA/Benzodiazepine Receptor Complex Antagonists

Dilantin and Flumazenil are given in a ratio of 50 mg/0.2 mg as a prophylaxis against the risk or occurrence of seizures due to rapid benzodiazepine reversal in drug overdoses involving individuals with regular or habitual use of benzodiazepines. In the event of “status epilepticus” induced by rapid reversal of benzodiazepine overdose, a conversion to use of separate baseline reversal drug (e.g. MU+NS-A1 ARA+S-A1ARA) with IV Dilantin (5-15 mg/kg) with infusion rate NTE 50 mg/min due to risk of cardiac arrhythmia.

Romazicon (flumazenil) Injection, USP: GCA: FLUMAZENIL 0.2 MG may repeat Q 2-3″ 0.2-1 mg total administered IV, IN. Flumazenil Injection, USP is a benzodiazepine receptor antagonist. Chemically, flumazenil is ethyl 8-fluoro-5,6-dihydro-5-methyl-6-oxo-4H-imidazo[1,5-a](1,4) benzodiazepine-3-carboxylate. Flumazenil has an imidazobenzodiazepine structure, a calculated molecular weight of 303.3, and the following structural formula:

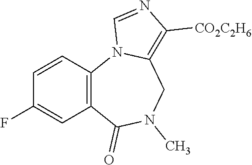

Flumazenil is a white to off-white crystalline compound with an octanol: buffer partition coefficient of 14 to 1 at pH 7.4. It is insoluble in water but slightly soluble in acidic aqueous solutions. Flumazenil injection is available as a sterile parenteral dosage form for intravenous administration. Each mL contains 0.1 mg of flumazenil compounded with 1.8 mg of methylparaben, 0.2 mg of propylparaben, 0.9% sodium chloride, 0.01% edetate disodium, and 0.01% acetic acid; the pH is adjusted to approximately 4 with hydrochloric acid and/or, if necessary, sodium hydroxide.

For the reversal of the sedative effects of benzodiazepines administered for conscious sedation, the recommended initial adult dose of flumazenil injection is 0.2 mg (2 mL) administered intravenously over 15 seconds. If the desired level of consciousness is not obtained after waiting an additional 45 seconds, a second dose of 0.2 mg (2 mL) can be injected and repeated at 60-second intervals where necessary (up to a maximum of 4 additional times) to a maximum total dose of 1 mg (10 mL). The dosage should be individualized based on the patient's response, with most patients responding to doses of 0.6 mg to 1 mg. In the event of re-sedation, repeated doses may be administered at 20-minute intervals as needed. For repeat treatment, no more than 1 mg (given as 0.2 mg/min) should be administered at any one time, and no more than 3 mg should be given in any one hour. It is recommended that flumazenil injection be administered as the series of small injections described (not as a single bolus injection) to allow the practitioner to control the reversal of sedation to the approximate endpoint desired and to minimize the possibility of adverse effects.

### (j) Mu and Opioid Subtype Receptor Agonists

Mu and opioid receptor subtype agonists are used for instance in transdermal patch embodiments that also include an α1-adrenergic receptor antagonist to prophylax against chest wall rigidity and/or a respiratory accelerant and/or a cholinergic agonist/antagonist to prevent or limit respiratory depression. Examples of mu receptor agonists include Fentanyl, Sufentanil, and Alfentanil. By way of example, dosages include: 0.1-1 mg of prazosin/50-100 mcg of fentanyl/pilocarpine 1-5 mg/(and/or) atropine 0.5-3 mg

Fentanyl: SUFENTA (fentanyl citrate) for Intravenous, Intramuscular, intranasal, INH or transdermal use. Fentanyl Citrate Injection is an opioid agonist. Fentanyl Citrate Injection, is a sterile, nonpyrogenic solution of fentanyl citrate in water for injection, available as 50 mcg (0.05 mg) per mL which is administered by the intravenous or intramuscular routes of injection. The chemical name is N-(1-phenethyl-4-piperidyl) propionanilide citrate (1:1). The molecular weight is 528.60; its molecular formula is C₂₂H₂₈N₂O.C₆H₈O₇. Fentanyl citrate, a white powder which is sparingly soluble in water. Each milliliter contains fentanyl (as the citrate) 50 mcg (0.05 mg). May contain sodium hydroxide and/or hydrochloric acid for pH adjustment. pH 4.7 (4.0 to 7.5). The molecular weight of fentanyl base is 336.5, and the empirical formula is C₂₂H₂₈N₂O. The n-octanol: water partition coefficient is 860:1. The pKa is 8.4. The chemical name is N-Phenyl-N-(1-(2-phenylethyl)-4-piperidinyl) propanamide. The structural formula is:

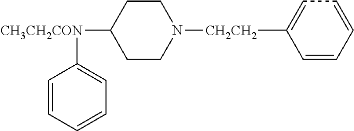

Fentanyl Citrate Injection should be administered only by persons specifically trained in the use of intravenous anesthetics and management of the respiratory effects of potent opioids. Ensure that an opioid antagonist, resuscitative and intubation equipment, and oxygen are readily available. Individualize dosage based on factors such as age, body weight, physical status, underlying pathological condition, use of other drugs, type of anesthesia to be used, and the surgical procedure involved. Monitor vital signs routinely. As with other potent opioids, the respiratory depressant effect of fentanyl may persist longer than the measured analgesic effect. Serious life-threatening respiratory failure (WCS) can occur with rapid injection.

SUFENTA® (sufentanil citrate) Injection. Note: Sufenta has similar dosing range as fentanyl, except usually at 1/10th the dose of fentanyl; thus, 5-10 mcg Sufenta=50-100 mcg Fentanyl. SUFENTA® (sufentanil citrate) is a potent opioid analgesic chemically designated as N-[4-(methyoxymethyl)-1-[2-(2-thienyl)ethyl]-4-piperidinyl]-N-phenyl-propanamide:2-hydroxy-1,2,3-propanetricarboxylate (1:1) with a molecular weight of 578.68. The structural formula of SUFENTA (sufentanil citrate injection) is:

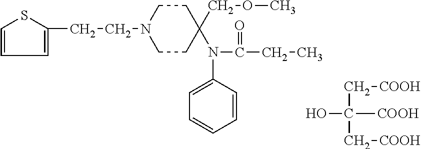

SUFENTA (sufentanil citrate injection) is a sterile, preservative free, aqueous solution containing sufentanil citrate equivalent to 50 μg per mL of sufentanil base for intravenous and epidural injection. The solution has a pH range of 3.5-6.0. The dosage of SUFENTA (sufentanil citrate injection) should be individualized in each case according to body weight, physical status, underlying pathological condition, and use of other drugs. In obese patients (more than 20% above ideal total body weight), the dosage of SUFENTA (sufentanil citrate injection) should be determined on the basis of lean body weight. Dosage should be reduced in elderly and debilitated patients. Vital signs should be monitored routinely.

SUFENTA (sufentanil citrate injection) may be administered intravenously by slow injection or infusion 1) in doses of up to 8 μg/kg as an analgesic adjunct to general anesthesia, and 2) in doses≥8 μg/kg as a primary anesthetic agent for induction and maintenance of anesthesia. If benzodiazepines, barbiturates, inhalation agents, other opioids or other central nervous system depressants are used concomitantly, the dose of SUFENTA and/or these agents should be reduced (see PRECAUTIONS). In all cases dosage should be titrated to individual patient response.

Alfentanil HCl Injection, USP Alfentanil HCl Injection, USP is an opioid analgesic chemically designated as N-[1-[2-(4-ethyl-4,5-dihydro-5-oxo1H-tetrazol-1-yl)ethyl]-4-(methoxymethyl)-4-piperidinyl]-N-phenylpropan-amide monohydrochloride (1:1) with a molecular weight of ˜452.98 and an n-octanol:water partition coefficient of 128:1 at pH 7.4. C₂₁H₃₂N₆O₃.HCl.H₂0. The structural formula of Alfentanil hydrochloride is:

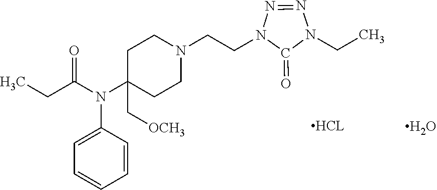 [SMILES: CCC(=O)N(c1ccccc1)C1(COC)CCN(CCn2nnn(CC)c2=O)CC1 ; O]

Alfentanil HCl Injection, USP is a sterile, non-pyrogenic, preservative free aqueous solution containing alfentanil hydrochloride equivalent to 500 mcg per mL of alfentanil base for intravenous injection. The solution, which contains sodium chloride for isotonicity, has a pH range of 4.0 to 6.0. In some instances, each mL contains: Active: Alfentanil base 500 mcg. Inactives: Sodium Chloride 9 mg and Water for Injection q.s. Alfentanil HCl injection is indicated as an analgesic adjunct given in incremental doses in the maintenance of general anesthesia; as a primary anesthetic agent for the induction of anesthesia in patients undergoing general surgery in which endotracheal intubation and mechanical ventilation are required. as the analgesic component for monitored anesthesia care (MAC). The dosage of Alfentanil HCl injection should be individualized and titrated to the desired effect in each patient according to body weight, physical status, underlying pathological condition, use of other drugs, and type and duration of surgical procedure and anesthesia. In obese patients (more than 20% above ideal total body weight), the dosage of Alfentanil HCl injection should be determined on the basis of lean body weight. The dose of Alfentanil HCl injection should be reduced in elderly or debilitated. Vital signs should be monitored routinely. Dosage should be individualized and titrated for use during general anesthesia.

Spontaneously breathing/assisted ventilation
Induction of Analgesia: 8 to 20 mcg/kg
Maintenance of Analgesia: 3 to 5 mcg/kg q 5 to 20 min or 0.5 to 1 mcg/kg/min
Total dose: 8 to 40 mcg/kg.

### (v) Compositions for Methods of Use.

The compounds disclosed herein can be formulated into compositions for direct administration to a subject for prophylaxis against or reversal of F/FA induced WCS. It is contemplated that the compounds may be administered to the same subject in concert, whether sequentially or simultaneously. The significant point regarding administration is that naloxone as a single agent, is ineffective and/or minimally effective in reversing the symptoms of WCS in humans and must be combined with other agents as noted in these compositions to be effective.

Specific combinations of compounds (Formula Equations) for use in several embodiments provided herein include the following [where MU=Mu receptor and/or opioid receptor subtype antagonists, MUXR=Extended release Mu receptor and/or opioid receptor subtype antagonists A1ARA=Alpha-1 Adrenergic receptor antagonist, A2ARA=Alpha-2 Adrenergic receptor agonist, VP=Vasopressor, AC=Anticholinergic, C=Cholinergic agent (nicotinic agonist and/or muscarinic agonist), PMR=Paralytic/Muscle relaxant, RA=Respiratory Accelerant (RA), GCA=GABA Complex Antagonist, and ASMS=Anti-seizure/Membrane stabilizer]:

Representative IMMEDIATE REVERSAL NON-MEDICAL (IRNM) embodiments:
(IRNM1) MU+S-A1ARA
(IRNM2) MU+A2ARA
(IRNM3) MU+NS-A1ARA
(IRNM4) MU+S-A1ARA+/−NS-A1ARA
(IRNM5) MU+S-A1ARA+/−NS-A1ARA+/−A2ARA
(IRNM6) MU+S-A1ARA+/−NS-A1ARA+/−AC or C
(IRNM7) MU+S-A1ARA+/−NS-A1ARA+/−AC or C+/−A2ARA

Representative IMMEDIATE REVERSAL MEDICAL NO AW (IRMnAW) embodiments: (including all of the previous embodiments in addition can be administered as an alternative to these formulations):
(IRMnAW1) MU+S-A1ARA+/−NS-A1ARA
(IRMnAW2) MU+S-A1ARA+/−NS-A1ARA+/−VP
(IRMnAW3) MU+S-A1ARA+/−NS-A1ARA+/−AC or C
(IRMnAW4) MU+S-A1ARA+/−NS-A1ARA+/−VP+AC or C
(IRMnAW5) MU+S-A1ARA+/−NS-A1ARA+/−RA
(IRMnAW6) MU+S-A1ARA+NS-A1ARA+/−VP+/−RA
(IRMnAW7) MU+S-A1ARA+NS-A1ARA+/−VP+RA+AC or C
(IRMnAW8) MU+S-A1ARA+NS-A1ARA+VP+RA+AC or C+A2ARA

Representative IMMEDIATE REVERSAL MEDICAL AW (IRMAW) embodiments (including all of the above previous embodiments in addition can be administered as an alternative to these formulations):
(IRMAW1) MU+S-A1ARA+/−NS-A1ARA
(IRMAW2) MU+S-A1ARA+/−NS-A1ARA+/−PMR
(IRMAW3) MU+S-A1ARA+NS-A1 ARA+/−VP+/−PMR
(IRMAW4) MU+S-A1ARA+NS-A1 ARA+/−PMR+/−AC or C
(IRMAW5) MU+S-A1ARA+NS-A1 ARA+/−VP+/−PMR+/−AC or C

Representative POLYSUBSTANCE (Poly) embodiments:
(Poly1) MU+S-A1 ARA+NS-A1ARA+GCA
(Poly2) MU+S-A1 ARA+NS-A1ARA+GCA+AC or C
(Poly3) MU+S-A1 ARA+NS-A1ARA+GCA+ASMS
(Poly4) MU+S-A1 ARA+NS-A1ARA+GCA+ASMS+AC or C
(Poly5) MU+S-A1 ARA+NS-A1ARA+GCA+ASMS+PMR
(Poly6) MU+S-A1 ARA+NS-A1ARA+GCA+ASMS+PMR+AC or C

Representative PROPHYLAXIS for ACTIVE OPIOID USER (PAOU) embodiments:
(PAOU1) S-A1ARA+VP
(PAOU2) S-A1ARA+/−AC or C
(PAOU3) S-A1ARA+VP+/−AC or C
(PAOU4) NS-A1ARA+VP
(PAOU5) NS-A1ARA+AC or C
(PAOU6) NS-A1ARA+VP+/−AC or C
(PAOU7) S-A1ARA+NS-A1ARA+/−VP+AC or C
(PAOU8) S-A1ARA+NS-A1ARA+/−AC or C
(PAOU9) S-A1ARA+NS-A1ARA+VP+AC or C

Representative PROPHYLAXIS for FIRST RESPONDERS (PFR) embodiments:
(PFR1) MU or MUXR+S-A1ARA+/−NS-A1ARA
(PFR2) MU or MUXR+S-A1ARA+NS-A1 ARA+/−AC or C
(PFR3) MU or MUXR+S-A1ARA+NS-A1 ARA+/−VP
(PFR4) MU or MUXR+S-A1ARA+NS-A1 ARA+VP+AC or C

(NOTE: cholinergic or anticholinergic agents for prophylaxis will be determined by baseline or resting cholinergic or parasympathetic tone as measured by heart rate and EKG QTc interval to rule out prolonged QTc and/or bradycardia or tachycardia at baseline.)

(NOTE: the addition of any anti-cholinergic or cholinergic agent in the above formulations will be at the discretion of the prescribing physician or trained medical professional administering these agents. Hemodynamics must be measured or assessed prior to either prescription or administration to avoid complications from administration. If the practitioner is uncertain regrading hemodynamics the baseline compound should be administered.)

Specific example dosage delivery systems are as follows: Intranasal (IN), sterile normal saline nasal solution (e.g., same % concentration and composition as standard 0.9% NaCl solution and pH adjusted to accommodate optimal solubility and deliverability of the molecules contained as solutes for delivery into the CNS); Intraocular (IOC), sterile normal saline or suitable ocular solution (e.g., % concentration, composition and pH adjusted to accommodate optimal solubility and deliverability of the molecules contained as solutes for delivery into the CNS); Intravenous (IV), sterile normal saline intravenous solution (e.g. same % concentration and composition as standard 0.9% NaCl solution); Intrathecal (IT), sterile isobaric, hypobaric and hyperbaric dextrose solutions for Intrathecal-CNS injection; Transdermal (TD), sterile slow release lipid matrix for transdermal absorption; intramuscular injection (IM), sterile slow release lipid matrix for intramuscular injection—IM and steady-state absorption; Intraosseus (10), sterile normal saline intravenous solution (e.g. same % concentration and composition as standard 0.9% NaCl solution); sublingual formulation (e.g. rapid dissolving tablet or strip) Oral formulation (e.g. capsule, tablet or gel cap); transtracheal atomization—sterile normal saline intravenous solution (e.g. same % concentration and composition as standard 0.9% NaCl solution); nebulizer—sterile normal saline intravenous solution (e.g. same % concentration and composition as standard 0.9% NaCl solution); and Metered dose inhaler (MDI). Thus, in various embodiments administration is via oral, sublingual—SL, intravenous—IV, intramuscular—IM, transdermal—TD, nasal insufflation—NI, inhalation—MDI, intraosseous injection—IO, intrathecal—IT injection, transtracheal—TT injection or atomization or intraocular—IO.

In particular embodiments, the therapeutic compounds are provided as part of composition that can include at least 0.1% w/v or w/w of therapeutic compounds; at least 1% w/v or w/w of therapeutic compounds; at least 10% w/v or w/w of therapeutic compounds; at least 20% w/v or w/w of therapeutic compounds; at least 30% w/v or w/w of therapeutic compounds; at least 40% w/v or w/w of therapeutic compounds; at least 50% w/v or w/w of therapeutic compounds; at least 60% w/v or w/w of therapeutic compounds; at least 70% w/v or w/w of therapeutic compounds; at least 80% w/v or w/w of therapeutic compounds; at least 90% w/v or w/w of therapeutic compounds; at least 95% w/v or w/w of therapeutic compounds; or at least 99% w/v or w/w of therapeutic compounds.

The compositions disclosed herein can be formulated for administration by, injection, inhalation, infusion, perfusion, lavage, topical ocular delivery or ingestion. The compositions disclosed herein can further be formulated for infusion via catheter, intravenous, intramuscular, intratumoral, intradermal, intraarterial, intranodal, intralymphatic, intraperitoneal, topical, intrathecal, intravesicular, oral and/or subcutaneous administration and more particularly by intravenous, intradermal, intraarterial, intranodal, intralymphatic, intraperitoneal, topical, intrathecal, intratumoral, intramuscular, intravesicular, oral and/or subcutaneous injection.

For injection and infusion, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions, and the like. For oral solid formulations such as, for example, powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For administration by inhalation, compositions can be formulated as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the therapeutic and a suitable powder base such as lactose or starch.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems utilizing semipermeable matrices of solid polymers containing at least one active ingredient. Various sustained-release materials have been established and are well known by those of ordinary skill in the art. Sustained-release systems may, depending on their chemical nature, release active ingredients following administration for two weeks to 1 month. In particular embodiments, a sustained-release system could be utilized, for example, if a human patient were to miss a weekly administration.

Specific expected formulations include those intended for immediate delivery, for instance where at least one (or each) component of the therapeutic system is provided in an immediate acting drug delivery system (for instance, IV, 10, CNS-Intrathecal injection, INH-metered dose inhaler, or Nasal spray administration). In other specific embodiments, the formulations include those intended for intermediate delivery, in which at least one (or each) component of the therapeutic system is provided in an intermediate acting delivery system. (for instance, oral extended release, or IM administration). In such intermediate delivery embodiments, onset generally in less than 1 hour, and duration is generally for up to 48 hours. Yet further embodiments provide extended release systems, for instance, extended release systems for prophylaxis. In such extended release systems, at least one (or each) component of the therapeutic system is provided in a long acting delivery system (for instance, slow release oral, extended release IM administration, or gel matrix patch). Onset for such extended release systems is generally within one hour or more, with resultant duration up to 60 days.

### (vi) Methods of Use.

Methods disclosed herein include treating subjects (including humans, veterinary animals, livestock, and research animals) with compositions disclosed herein. As indicated, the compositions can treat a variety of different conditions, including intentional or accidental exposure to and/or overdose with one or more opiate or opioid compounds, or a mixture containing at least one opiate or opioid compound; or one or more symptoms associated with opiate/opioid overdose (including but not limited to FIRMR, laryngospasm and/or WCS). Specific examples of methods of use, including clinical settings in which such use might occur, are provided in Table 1 and the text associated therewith, as well as the Examples.

Treating subjects includes delivering therapeutically effective amounts of one or more composition(s). Therapeutically effective amounts can provide effective amounts, prophylactic treatments, and/or therapeutic treatments.

An “effective amount” is the amount of a compound necessary to result in a desired physiological change or effect in the subject. Effective amounts disclosed herein result in partial or complete reversal or prevention of a symptom of opiate/opioid exposure or overdose following administration to a subject.

A “prophylactic treatment” includes a treatment administered to a subject who does not display signs or symptoms of a condition or displays only early signs or symptoms of the condition such that treatment is administered for the purpose of diminishing, preventing, or decreasing the risk of developing the condition further or in anticipation of exposure to the toxin or offensive chemical agent. Thus, a prophylactic treatment functions as a preventative treatment.

A “therapeutic treatment” includes a treatment administered to a subject who displays symptoms or signs of a condition and is administered to the subject for the purpose of diminishing or eliminating one or more of those signs or symptoms of the condition.

Prophylactic and therapeutic treatments need not fully prevent or cure a condition but can also provide a partial benefit.

One embodiment of the method involves use of a Mu opioid receptor and/or opioid receptor subtype (mu, kappa, delta receptor subtypes) antagonist (e.g. naloxone) in combination with an Alpha-adrenergic receptor antagonist—AARA (e.g. prazosin, terazosin, tamsulosin, doxazosin) and/or a cholinergic agent (muscarinic receptor antagonist/anticholinergic, M3 receptor agonist or a nicotinic receptor general or selective agonist) for immediate reversal of FIRMR, laryngospasm and/or WCS and overdose related to F/FAs or F/FAs combined with morphine or morphine derivatives.

Another embodiment of the method as an opioid analgesic involves use of a piperidine derived mu receptor agonist in combination with, but not limited to, an α-adrenergic receptor antagonist—AARA and/or a cholinergic agent (muscarinic receptor antagonist/anticholinergic, M3 receptor agonist or a nicotinic receptor general or selective agonist) and/or a respiratory accelerant (e.g. as a therapeutic compound for analgesia that is now prophylaxed against the occurrence of FIRMR, laryngospasm and/or WCS (e.g. transdermal fentanyl patch combined with an α adrenergic antagonist and possibly naltrexone or naloxone particles that are not bioactive unless the gel matrix is disrupted by tampering) for analgesia with WCS prophylaxis.

Another embodiment of the method involves use of a mu opioid receptor and/or opioid receptor subtype (mu, kappa, delta receptor subtypes) antagonist in combination with an α-adrenergic receptor antagonist—AARA, a centrally acting respiratory center stimulant (e.g. doxapram hydrochloride, almitrine) and/or a cholinergic agent (muscarinic receptor antagonist/anticholinergic, M3 receptor agonist or a nicotinic receptor general or selective agonist) for immediate reversal with increased respiratory drive.

Another embodiment of the method involves use of a mu opioid receptor and/or opioid receptor subtype (mu, kappa, delta receptor subtypes) antagonist in combination with an α-adrenergic receptor antagonist and/or an alpha-2 adrenergic receptor agonist and/or a vasoactive agent (e.g. phenylephrine, ephedrine, epinephrine) to offset hypotension and for immediate reversal with a clinical presentation of hypotension.

Another embodiment of the method involves use of mu opioid receptor and/or opioid receptor subtype (mu, kappa, delta receptor subtypes) antagonist in combination with an α-adrenergic receptor antagonist and an anticholinergic agent and/or a cholinergic agent (muscarinic receptor antagonist/anticholinergic, M3 receptor agonist or a nicotinic receptor general or selective agonist) (e.g. glycopyrrolate, atropine) to offset bradycardia and for immediate reversal with a clinical presentation of bradycardia or asystole or laryngospasm or upper airway effects.

Another embodiment of the method involves use of a mu opioid receptor and/or opioid receptor subtype (mu, kappa, delta receptor subtypes) antagonist in combination with an α-adrenergic receptor antagonist—AARA and a rapid acting muscle paralytic (e.g. succinylcholine, rocuronium) to synergistically interact with AARA to reduce or reverse FIRMR, laryngospasm and/or WCS and for immediate reversal with a clinical presentation of severe or persistent respiratory muscle rigidity and/or laryngospasm.

Another embodiment of the method involves use of a mu opioid receptor and/or opioid receptor subtype (mu, kappa, delta receptor subtypes) antagonist in combination with an α-adrenergic receptor antagonist, a vasoactive agent (e.g. phenylephrine, ephedrine, epinephrine) to offset hypotension and an anticholinergic to either decrease bradycardia induced by phenylephrine or amplify or reinforce the effects of ephedrine on heart rate and for immediate reversal with a clinical presentation of hypotension and bradycardia or asystole.

Another embodiment of the method involves use of a mu opioid receptor and/or opioid receptor subtype (mu, kappa, delta receptor subtypes) antagonist in combination with an α-adrenergic receptor antagonist and an anticholinergic agent (e.g. glycopyrrolate, atropine) to offset bradycardia and for immediate reversal with a clinical presentation of bradycardia.

One embodiment of the method involves use of an α-adrenergic receptor antagonist—AARA and a vasoactive agent (e.g. phenylephrine, ephedrine, epinephrine) to offset hypotension for prophylaxis in a population at risk for FIMR/FIRMR/WCS due to habitual use or exposure to prescribed, illicit or IV, insufflated F/FAs.

One embodiment of the method involves use of an α-adrenergic receptor antagonist—AARA and an anticholinergic agent and/or a cholinergic agent (muscarinic receptor antagonist/anticholinergic, M3 receptor agonist or a nicotinic receptor general or selective agonist) (e.g. glycopyrrolate, atropine) to offset bradycardia, laryngospasm and decrease vagal tone as prophylaxis in a population at risk FIRMR/WCS due to habitual use or exposure to prescribed, illicit, IV, INH or insufflated F/FAs.

One embodiment of the method involves use of an α-adrenergic receptor antagonist—AARA, an α-1B agonist-vasoactive agent (e.g. phenylephrine) to offset hypotension for prophylaxis and an anticholinergic agent (e.g. glycopyrrolate, atropine) to offset bradycardia induced by phenylephrine in a population at risk for FIMR due to habitual use or exposure to prescribed, illicit, IV, IM, INH or insufflated F/FAs.

Another embodiment of the method involves use of an extended-release mu opioid receptor and/or opioid receptor subtype (mu, kappa, delta receptor subtypes) antagonist (e.g. naltrexone) in combination with an α-adrenergic receptor antagonist and a vasoactive agent (e.g. phenylephrine, ephedrine, epinephrine) to offset hypotension and for prophylaxis against FIMR in a population at risk for environmental exposure to F/FAs.

Another embodiment of the method involves use of an extended-release mu opioid receptor antagonist (e.g. naltrexone) and/or opioid receptor subtype (mu, kappa, delta receptor subtypes) antagonist in combination with an α-adrenergic receptor antagonist and an anticholinergic agent and/or a cholinergic agent (muscarinic receptor antagonist/anticholinergic, M3 receptor agonist or a nicotinic receptor general or selective agonist) (e.g. glycopyrrolate, atropine) to offset bradycardia, laryngospasm and alter vagal tone as prophylaxis against FIRMR/WCS in a population at risk for environmental exposure to F/FAs.

One embodiment of the method involves use of an α-adrenergic receptor antagonist—AARA, an α-1B agonist-vasoactive agent (e.g. phenylephrine) to offset hypotension for prophylaxis and an anticholinergic agent (e.g. glycopyrrolate, atropine) to offset bradycardia induced by phenylephrine, in a population at risk for FIMR from environmental exposure to F/FAs.

Another embodiment of the method involves use of an extended-release mu receptor antagonist and/or an opioid receptor subtype (mu, kappa, delta receptor subtypes) antagonist (e.g. naltrexone) in combination with an α-adrenergic receptor antagonist (e.g. a centrally acting and/or a peripherally acting agent) and a vasoactive agent (e.g. phenylephrine, ephedrine, epinephrine). Optionally, an anticholinergic agent and/or a cholinergic agent (muscarinic receptor antagonist/anticholinergic, M3 receptor agonist or a nicotinic receptor general or selective agonist) (e.g. glycopyrrolate, atropine) can also be administered, to offset hypotension, bradycardia, laryngospasm and alter vagal tone as prophylaxis against FIRMR/WCS in a population at risk for environmental exposure to F/FAs or a population of opioid users in recovery with risk of relapse.

Another embodiment of the method involves use of an extended-release mu opioid receptor antagonist and/or another opioid receptor subtype (mu, kappa, delta receptor subtypes) antagonist (e.g. naltrexone) in combination with an α-adrenergic receptor antagonist (e.g. a selective AARA and a non-selective AARA) and a vasoactive agent (e.g. phenylephrine, ephedrine, epinephrine) to offset hypotension and for prophylaxis against FIRMR/WCS in a population at risk for environmental exposure to F/FAs.

One embodiment of the method involves use of an α-adrenergic receptor antagonist—AARA (e.g. a selective AARA and a non-selective AARA), an α-1B agonist-vasoactive agent (e.g. phenylephrine) to offset hypotension for prophylaxis and an anticholinergic agent (e.g. glycopyrrolate, atropine) to offset bradycardia induced by phenylephrine in a population at risk for FIRMR/WCS due to habitual use or exposure to prescribed, illicit, IV, INH, IM or insufflated F/FAs.

One embodiment of the method involves use of an α-adrenergic receptor antagonist—AARA (e.g. a selective AARA and a non-selective AARA), an α-1B agonist-vasoactive agent (e.g. phenylephrine) to offset hypotension for prophylaxis and an anticholinergic agent (e.g. glycopyrrolate, atropine) to offset bradycardia induced by phenylephrine in a population at risk for FIRMR/WCS due to habitual use or exposure to prescribed, illicit, IV, INH, IM or insufflated F/FAs.

One embodiment of the method involves use of an α-adrenergic receptor antagonist—AARA (e.g. a selective AARA and a non-selective AARA), an α-1B agonist-vasoactive agent (e.g. phenylephrine) to offset hypotension for prophylaxis and an anticholinergic agent (e.g. glycopyrrolate, atropine) to offset bradycardia induced by phenylephrine, in a population at risk for FIRMR/WCS from environmental exposure to F/FAs.

(vii) Kits.

Combinations of active components (including specifically synergistic combinations) can be provided as kits. Kits can include containers including one or more or more compounds as described herein, optionally along with one or more agents for use in combination therapy. For instance, some kits will include an amount of at least one α-adrenergic receptor antagonist (for instance, a centrally acting or peripherally acting α-adrenergic receptor antagonist or agonist, or a combination thereof), along with an amount of at least one Mu opioid receptor antagonist and/or another opioid receptor subtype (mu, kappa, delta receptor subtypes) antagonist (for instance, a long-acting Mu receptor antagonist), a centrally-acting or peripherally acting respiratory stimulant, a GABA/benzodiazepine receptor complex antagonist, an α2-adrenergic receptor agonist, a Mu receptor agonist, vasoactive agents, anticholinergic agents and/or cholinergic agents (muscarinic receptor antagonist/anticholinergic, M3 receptor agonist or a nicotinic receptor general or selective agonist).

Specific contemplated kits included kits tailored to the user of the kit, for instance, an untrained provider kit, a medically trained provider kit (which for instance, may include a vital sign algorithm dosing chart), an emergency administration kit, and so forth. Table 1 provides information regarding types of compounds (and representative compounds) that would be included in certain different kit types.

Similarly, different kits may be provided for different routes of delivery, including for IV, IM, IN, 10, IT, IOC, and TT delivery.

Any active component in a kit may be provided in premeasured dosages, though this is not required; and it is anticipated that certain kits will include more than one dose, including for instance when the kit is used for a method requiring administration of more than one dose of the synergistic combination.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding preparation of component(s) of the synergistic combination, for administration; proper disposal of related waste; and the like. The instructions can be in the form of printed instructions provided within the kit or the instructions can be printed on a portion of the kit itself. Instructions may be in the form of a sheet, pamphlet, brochure, CD-ROM, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made. The instructions of the kit will direct use of the active ingredients to effectuate a clinical use described herein. In effect, this document offers instruction in the formulation of compounds and the administration of these compounds for the treatment of (prophylaxis or reversal) WCS and other respiratory and muscular effects of F/FAs and morphine derived alkaloids.

### EXEMPLARY EMBODIMENTS

The following examples are provided to illustrate certain particular features and/or embodiments. These examples should not be construed to limit the disclosure to the particular features or embodiments described.

The herein provided technology has three general modes of use:
1. Immediate opioid reversal treatment for someone who has overdosed on F/FAs, opioids, or a combination of morphine derived opiates combined with F/FAs. Optionally, the immediate reversal composition also includes drug(s) of the benzodiazepine class and is categorized as “polysubstance” reversal.
2. Prophylaxis treatment for someone who is likely to have exposure to F/FAs, for instance by environmental exposure, or by intentional/unintentional use of IV opioids or over-ingestion of opioids containing fentanyl or fentanyl analogues or F/FAs combined with a morphine derived opiate.
3. F/FAs with modified side effect SE profiles: F/FA based analgesia can be provided to a subject with acute or chronic pain whereby the F/FA is combined with simultaneously released agents that serve to limit the side effect profile of the opioid (e.g. respiratory depression, laryngospasm, FIRMR, WCS, addiction etc.) and thereby enhance or increase the safety margin and potential for extended ranges of analgesia.

In each general mode of use, the use of compounds (and the corresponding preparations to be used in such methods) is further subdivided according to the (known or expected) baseline skill set of the “provider” or “responder” as either being “non-medical”, “medical provider without AW training or”, “medical provider with AW training.” The assignment of possible compounds that can be used by each type of provider is made according to skill set and clinical presentation for the discernment of the medically trained providers. Examples 2-6 below provide description of how such different responders might use composition(s) provided herein in a method of reversing or preventing one or more effects of opioid/opiate dosing, largely in the format that may be used for packaging instructions or other product-associated literature.

### Example 1: Production of Baseline Formulation Doses

This example describes representative dosage amounts of compounds for use in combination therapies described herein. Lower doses can be employed, but improvement of clinical outcome is less likely to be affected or effective at lower doses. Similarly, higher doses can be used, but can negatively impact the overall clinical outcome and survival rates. The baseline formulation doses are designed so that the initial dose can be elevated proportionally by administering additional doses until FIRMR/WCS or overdose condition is reversed or stabilized. In many situations, 1-4 doses will be sufficient for treatment, but the number and size of dose can be modified to accommodate severe or persistent symptoms from overdose. The chart below for BASE DOSE COMPOUND (BDC) is a guide and is not meant to be limited to dose examples, route and ranges listed below.

TABLE 2
BASE DOSE COMPOUND (BDC), assuming 70 kg adult (±10 kg):

| | Representative | | | |
|---|---|---|---|---|
| Class | Compound(s) | BDC-Ideal Dose | Timing | Dose Range/Route |
| MU | NALOXONE | 1 MG | May repeat Q 2-3” | 0.2-4 mg total |
| | | (14 mcg/kg) | | IV, IM, IN, IO, IOC |
| | NALTREXONE | 25 MG | QD | 25-50 mg total |
| | | | | IV, IM, PO, IN, IO, |
| | | | | IOC |
| | NALMEFENE | 0.4-1 MG | May repeat Q 2-3” | NTE 1.5 mg total |
| | | (20-40 mcg/kg) | | IV, IM, IN, IO, IOC |
| A1ARA | NS-PRAZOSIN | 0.25-0.5 MG* | May repeat Q 2-3” | 0.1-20 mg total |
| | | (3-7 mcg/kg) | | IV, IM, IN, IO, IOC |
| | S-TAMSULOSIN | 0.2-0.4 MG | May repeat Q 2-3” | 0.1-0.8 mg total |
| | | 3-6 mcg/kg) | | IV, IM, IN, IO, IOC |
| | TERAZOSIN | 1 MG | May repeat Q 2-3” | 0.5-5 MG NTE 5 MG |
| | | | | total |
| | | | | IV, IM, IN, IO, IOC |
| AC | ATROPINE | 0.5-1 MG | May repeat Q 2-3” | 5-40 mcg/kg |
| | | | | or 3 mg total |
| | | | | IV, IM, IN, IO, IOC |
| | GLYCOPYRROLATE | 0.2 MG | May repeat Q 2-3” | NTE 0.1-0.5 mcg/kg |
| | | | | 1.5 mg total |
| | | | | IV, IM, IN, IO, IOC |
| PMR | SUCCINYLCHOLINE | 1-3 MG | May repeat Q 2-3” | 0.5-1.1 mg/kg |
| | | (14-28 mcg/kg) | | IV, IM, IN, IO |
| RA | DOXAPRAM | 10 MG | May repeat Q 2-3” | 0.1-1 mg/kg |
| | | | | 40 mg total |
| | | | | IV, IM, IN, IO, IOC |
| VP | EPHEDRINE | 5-10 MG | May repeat Q 2-3” | 1-20 mg |
| | | std conc. at 10 mg | | IV, IM, IN, IO, IOC |
| | PHENYLEPHRINEΔ | 10-100 MCG | May repeat Q 2-3” | 25-200 mcg/min |
| | | std conc. at 50 mcg | | IV, IN |
| | EPINEPHRINE§ | See legend and | | |
| | | refer to ACLS | | |
| | | dosing standards. | | |
| GCA | FLUMAZENIL | 0.2 MG ** | May repeat Q 2-3” | 0.2 mg-1 mg |
| | | | | IV, IM, IN, IO, IOC |
| ASMS | DILANTIN | 50 MG*** | May repeat Q 2-3” | NTE 50 mg/min rapid |
| | | | | IV infusion |
| | | | | IV, IN, IO, IOC |
| COMBO | DROPERIDOL∴ | 0.5 MG | May repeat Q 2-3” | 0.01-0.25 mg/kg IV, |
| (AARA-AC | | | 1-4 doses of BDC | IM or IN, IOC |
| or | | | | |
| “COMBO”) | | | | |
| “C” | PILOCARPINE♦ | 1 MG | May repeat Q 2-3” | 1 mg (1- 2.5 mg : |
| (PILO) | | | 1-4 doses of BDC | NTE 10 mg ) |
| | | | | IV, IM or IN, IOC |

With regard to table 2, above:
*All of these drug, with the exception of the A1ARAs prazosin and tamsulosin (see “A1ARA IV/IN/IM formulation protocol”) are currently available as IV formulations and therefore can be easily converted to nasal dosing regimens, which are similar in potency and concentration, if not the same, and will be concentratable in a nasal, IV or IM formulation. Both prazosin and tamsulosin can be solubilized and made suitable for IV injection or IN insufflation by standard compounding pharmaceutical techniques.
**Dilantin and Flumazennil will be given in a ratio of 50 mg/0.2 mg as a prophylaxis against the risk or occurence or seizures due to rapid benzodiazepine reversal in drug overdoses involving individuals with regular or habitual use of benzodiazepines.
***In the event of “status epilepticus” induced by rapid reversal of benzodiazepine overdose, a conversation to use of seperate baseline reversal drug (e.g.., MU + NS-A1ARA) with IV Dilantin (5-15 mg/kg) with infusion rate NTE 50 mg/min due to risk of cardiac arrhythmia.
§Epinephrine is to be used with caution in individuals with F/FAs overdose due to the direct and potent activity of Epinephrine and Noradrenaline at the LC and FIMR/FIRMR/WCS related circuitry. However, should this be the initial presentation in “Suspected Opioid Overdose”, the medical practitioner should use their discretion to follow best practices and go directly to the most current ACLS treatment algorithms with the possible addition of the “Baseline formulation” for FIMR reversal. The ACLS dose protocol for cardiac arrest-1 mg IV and may repeat Q2-3” for total dose of 3 mg or Infusion 1 mg EPINEPHRINE in 250 ml of D5W (4 mcg/ml) IV infusion rate NTE (1-4 mcg/min).
ΔPhenylephrine may be bolused 10-200 mcg IV or may be given via IV infusion 20 mg of Phenylephrine in 250 ml of D5W (5% dextrose in sterile water) (80 mcg/ml) IV infusion rate NTE (25-200 mcg/min).
∴Droperidol (combined alpha-1 adrenergic antagonist and anticholinergic-AARA-AC) can be administered in a dose range of 0.01-0.25 mg/kg IV, M or IN. Dosing at higher ranges is known to be associated with increased risk of cardiac arrhythmias (SEE black box warning label), however is rare in occurrence. Initial doses of up to 2.5 mg are well tolerated with additional doses of 0.5-1.25 mg may be administered if benefit of F/FA overdose or toxic exposure reversal outweighs potential risk of upper dose range.
♦Pilocarpine (M3/muscarinic agonist) anticholinergic agents and/or cholinergic agents (muscarinic receptor antagonist/anticholinergic, M3 receptor agonist or a nicotinic receptor general or selective sgonist).

Formulation Key: (Therapeutic Classes and Abbreviations Used Below)
1) Mu receptor antagonists (MU) (e.g. naloxone, naltrexone) Each member of this class has an accompanying designation indicating whether they are immediate acting or extended release (XR) (e.g. naltrexone is a long acting MU antagonist, MUXR). Also note that this class can contain selective opioid receptor antagonists and agonists for kappa and delta subtypes.
2) A-1 Adrenergic receptor antagonists (A1ARA) (e.g. prazosin, tamsulosin) Each member of this class has an accompanying designation indicating whether they are selective (S) or non-selective (NS) for A1ARA subtypes 1A, 1B, or 1D (e.g. Selective A 1A receptor antagonist tamsulosin would be designated as S-A1 ARA).
3) Vasopressors (VP) (e.g. phenylephrine, ephedrine)
4) Anticholinergics (AC) (e.g. atropine, glycopyrrolate)
5) Paralytics/Muscle relaxants (PMR) (e.g. succinylcholine)
6) Respiratory Accelerants (RA) (e.g. doxapram)
7) GABA Complex Antagonists (GCA) (e.g. flumazenil)
8) Anti-seizure/Membrane stabilizer (ASMS) (e.g. Dilantin XR)
9) Fentanyl/Fentanyl analogues (F/FAs) (e.g. fentanyl, sufentanil, alfentanil)
10) Alpha2 agonists (A2A) (e.g., Clonidine)
11) Alpha1 agonists (A1A) (e.g. phenylephrine—also listed as a “vasopressor” above)
12) Anticholinergic (AC) agents and/or cholinergic agents (C) (muscarinic receptor antagonist/anticholinergic, M3 receptor agonist or a nicotinic receptor general or selective agonist) (e.g. Pilocarpine)
13) Combined alpha-1 adrenergic antagonist and anticholinergic (AARA-AC or “COMBO”) (e.g. Droperidol)

Specific combinations of compounds (Formula Equations) for use in embodiments provided herein include the following: Representative IMMEDIATE REVERSAL NON-MEDICAL embodiments include: IRNM1, IRNM2, IRNM3, IRNM4, IRNM5, IRNM6, and IRNM7. Representative IMMEDIATE REVERSAL MEDICAL NO AW embodiments include: IRMnAW1, IRMnAW2, IRMnAW3, IRMnAW4, IRMnAW5, IRMnAW6, IRMnAW7, and IRMnAW8. Representative IMMEDIATE REVERSAL MEDICAL AW embodiments (these personnel can also employ formulations listed in MEDICAL NO AW) include: IRMAW1, IRMAW2, IRMAW3, IRMAW4, and IRMAW5. Representative POLYSUBSTANCE embodiments include: Poly1, Poly2, Poly3, Poly4, Poly5, and Poly6. Representative PROPHYLAXIS for ACTIVE OPIOID/IV USER embodiments include: PAOU1, PAOU2, PAOU3, PAOU4, PAOU5, PAOU6, PAOU7, PAOU8, and PAOU9. Representative PROPHYLAXIS for FIRST RESPONDERS embodiment include: PFR1, PFR2, PFR3, and PFR4.

### Example 2: Methods for Fentanyl/Fentanyl Analog Overdose Treatment, Non-Medical Provider

The following combinations of therapeutic agents are appropriate for use by non-medically trained persons in an immediate reversal situation: IRNM1, IRNM2, IRNM3, IRNM4, IRNM5, IRNM6, and IRNM7.

Representative Delivery systems for non-medical and medical providers: (e.g., intranasal and intramuscular injection). This description is intended to illustrate and be informative, but is not intended to be comprehensive regarding the scope of resuscitation from opioid overdose, or regarding more sophisticated airway and cardiovascular treatment algorithms.

FIRMR/WCS REVERSAL AGENTS as a Nasal Spray is a prescription medicine used for the treatment of an opioid emergency such as an overdose or a possible suspected opioid overdose where fentanyl or fentanyl analogues are involved or if either of these opioid drugs are combined with morphine derivatives and present with signs of breathing problems, sudden onset of muscle rigidity in chest wall, upper extremities and/or abdomen or “seizure-like”, rapid loss of consciousness, severe sleepiness, body found with syringe or tourniquet still in place/injection site, rapid onset of cyanosis, pinpoint pupils, or not being able to respond after an injection of illicit drugs or unintentional ingestion of fentanyl or fentanyl analogues.

FIRMR/WCS REVERSAL AGENTS—Nasal Spray is to be given right away, but does not take the place of emergency medical care. Get emergency medical—EMS CALL 911—help right away after giving the first dose of FIMR REVERSAL AGENTS Nasal Spray, even if the person wakes up. The opioid effects often outlast the effect of the mu antagonist agent unless it is long acting. FIRMR/WCS REVERSAL AGENTS—Nasal Spray can be safe and effective in children for known or suspected opioid overdose however, but always refer to the package insert for dosing guidelines and call EMS-911 immediately. FIRMR/WCS REVERSAL AGENTS Nasal Spray is used to temporarily reverse the effects of opioid medicines and specifically opioid overdoses that involve fentanyl and fentanyl analogues. The medicine in FIMR REVERSAL AGENTS Nasal Spray has little effect in people who are not taking opioid medicines, but can either raise or lower blood pressure and repeat dosing should be done with caution only in a witnessed overdose or wait till skilled emergency providers arrive. Always carry FIMR REVERSAL AGENTS Nasal Spray with you in case of an opioid emergency. Use FIRMR/WCS REVERSAL AGENTS Nasal Spray right away if you or your caregiver think signs or symptoms of an opioid emergency are present, even if you are not sure, because an opioid emergency can cause severe injury or death.

REVERSAL AGENTS—Nasal Spray. Rescue breathing or CPR (cardiopulmonary resuscitation) and BLS (basic life support) may be given while waiting for emergency medical help.

The signs and symptoms of an opioid emergency can return after FIRMR/WCS REVERSAL AGENTS—Nasal Spray is given. If this happens, give another dose after 2 to 3 minutes using a new FIRMR/WCS REVERSAL AGENTS—Nasal Spray and closely watch the person until emergency help is received.

HOW TO USE FIRMR/WCS REVERSAL AGENTS—Nasal Spray as delivery system

In opioid overdose emergencies recognize symptoms and taking prompt action is critical to potentially saving a life. If you suspect an opioid overdose, administer FIRMR/WCS REVERSAL AGENTS—Nasal Spray and get emergency medical assistance right away. Key steps to administering FIMR reversal agents—Nasal Spray:
1. Peel back the package to remove the device. Hold the device with your thumb on the bottom of the plunger and two fingers on the nozzle.
2. Place and hold the tip of the nozzle in either nostril until your fingers touch the bottom of the patient's nose.
3. Press the plunger firmly to release and inject the dose into the patient's nose.

The FIRMR/WCS REVERSAL DRUG-Auto-Injector is a disposable, pre-filled automatic injection device to be used in the event of an opioid emergency such as an overdose or a possible suspected opioid overdose where fentanyl or fentanyl analogues are involved or if either of these opioid drugs are combined with morphine derivatives. FIRMR/WCS REVERSAL DRUG-Auto-Injector administers NALOXONE and an ALPHA-1 ADRENERGIC RECEPTOR ANTAGONIST (in one specific example). Auto-injectors may be color-coded or otherwise readily labeled to acknowledge that they may contain other combinations of medications (see Table 1) to be used at the discretion of a medical provider and are to be used in the event of an opioid overdose where fentanyl or a fentanyl analogues are suspected. FIRMR/WCS REVERSAL DRUG-Auto-Injector is a prescription medicine used for the treatment of an opioid emergency such as an overdose or a possible suspected opioid overdose where fentanyl or fentanyl analogues are involved and present with signs as noted above in “Delivery systems for non-medical and medical providers”.

FIRMR/WCS REVERSAL DRUG-Auto-Injector is used to temporarily reverse the effects of opioid medicines and specifically opioid overdoses that involve fentanyl and fentanyl analogues. The medicine in FIRMR/WCS REVERSAL DRUG-Auto-Injector has little effect in people who are not taking opioid medicines, but can lower blood pressure and repeat dosing should be done with caution only in a witnessed overdose or wait till skilled emergency providers arrive. In the meantime, provide CPR and BLS support until emergency providers arrive. Get emergency medical help right away after giving the first dose of FIRMR/WCS REVERSAL DRUG-Auto-Injector Rescue breathing or CPR (cardiopulmonary resuscitation) and BLS (basic life support) may be given while waiting for emergency medical help. The signs and symptoms of an opioid emergency can return after FIMR REVERSAL DRUG-Auto-Injector is given. If this happens, give another dose after 2 to 3 minutes using a new FIMR REVERSAL DRUG-Auto-Injector and closely watch the person until emergency help is received.

### Example 3: Methods for Fentanyl/Fentanyl Analog Overdose Treatment, Medical Provider

The technology provided herein is designed to accommodate multiple types of first responders with different skill sets and training. TABLE 1 and the Formula Equations provided herein identify and assign combination compounds to each type of provider, including by clinical presentation. These FIRMR/WCS REVERSAL DRUGS should be combined in effect with standard BLS/CPR/ACLS protocols to manage the effects of opioid overdose and used to temporarily reverse the effects of opioid medicines and specifically opioid overdoses that involve fentanyl and fentanyl analogues.

The following are exemplary situations in which a Provider who has medical training can administer the indicated combination therapy:
1) Suspected opioid OD, unresponsive patient with rapid, “thready” pulse indicating low BP: Use formulation with Naloxone, Prazosin OR A-1A selective antagonist and Phenylephrine.
2) Suspected opioid OD with slow, “thready” pulse and low BP: Use formulation with Naloxone, Prazosin or A-1A selective antagonist, Ephedrine and glycopyrrolate and/or atropine.
3) Suspected opioid OD with prominent rigidity: Use formulation with Naloxone, Prazosin or A-1A selective antagonist and glycopyrrolate and/or atropine or a selective M3 agonist to block F/FA muscarinic antagonist effects and/or Succinylcholine or Rocuronium (e.g. only if medical provider has advanced AW management training and all necessary equipment available to secure the AW).
4) Suspected opioid OD is pulseless and low pressure: Use formulation with Naloxone, prazosin, ephedrine, glycol/atropine, epinephrine.
5) Uncertain presentation: In the event that the medical provider is uncertain of physiologic or clinical presentation in “Suspected Opioid Overdose”, use the baseline “Non-Medical” dosing kit until vital signs are apparent and directional, then follow algorithm as above in clinical scenarios 1-4.

More generally, the following is a list of preparations (that is, combinations of compounds) that are applicable for use by a medically trained Provider—with hemodynamic monitoring available and with or without airway monitoring/equipment available (“Immediate Reversal Medical No AW”) or with the ability to monitor and equipment to manage airway function (“Immediate Reversal Medical AW”; can also employ all formulations listed in Immediate Reversal Medical No AW):
Immediate Reversal Medical No AW-composition combinations: IRMnAW1, IRMnAW2, IRMnAW3, IRMnAW4, IRMnAW5, IRMnAW6, IRMnAW7, and IRMnAW8
Immediate Reversal Medical AW-composition combinations: IRMAW1, IRMAW2, IRMAW3, IRMAW4, and IRMAW5

In each clinical presentation scenario, after administering a drug, the Medical provider should continue to provide CPR/ACLS and continue to reassess the patient every 1-2″ for response to the last drug given and assess clinical presentation for the next type of dose to be given. Re-dosing of drug combination can be done every 2-3 minutes (2-3″) up to four doses, or more if the patient is responding, but still needs additional reversal.

The clinical scenarios provided in “TABLE 1” and listed above serve as the guidelines for continued dosing strategies (e.g., if the pulse is slow HR<60, use the composition that contains ATROPINE or GLYCOPYRROLATE).

If blood pressure is low Systolic<90 and Diastolic<50 and heart rate<60, use the compound with ATROPINE and Glycopyrrolate and the vasopressor EPHEDRINE because it will increase BP and also increase HR.

If the BP is low Systolic<90 and Diastolic<50, but the pulse is “thread” and fast HR>90, then use Phenylephrine because it will raise BP, but also lower HR simultaneously.

If patient presents with rigidity and the Medical provider has no AW experience, they would use the compound with ATROPINE and Glycopyrrolate and and/or a selective M3 agonist to block F/FA muscarinic antagonist effects and upper AW effects and/or the vasopressor EPHEDRINE to speed up the HR and decrease “vagal tone” which will help improve rigidity.

If the patient presents with extreme rigidity and the Medical provider has AW training and AW equipment available, then use the compound that contains SUCCINYLCHOLINE to break the rigidity). Alternatively, a mu opioid receptor antagonist can be combined with an alpha 1-adrenergic antagonist and an anticholinergic agent and/or a selective M3 agonist to block F/FA muscarinic antagonist effects and upper AW effects.

In each scenario, after administering a drug combination, the Medical provider should provide CPR/ACLS and continue to reassess the patient for response to the last dose given, and assess clinical presentation for the next type of medication to be given. The Medical provider with AW training has the most options available followed by the Medical provider with only hemodynamic training.

### Example 4: Methods for Polysubstance Overdose Treatment

In instances of suspected or known polysubstance overdose, treatment is carried out similarly to the description provided in the prior examples but using one of the following combined therapeutic compositions: Poly1, Poly2, Poly3, Poly4, Poly5, and Poly6

### Example 5: Methods of Prophylaxis for Habitual Drug Users

In embodiments used to provide prophylaxis for habitual drug uses, dosing regimens, formulas, and general instructions are largely as presented in prior Examples. However, combinations of compounds for these embodiments have been modified to exclude mu antagonists. This is because habitual users of illicit opiates or individuals with a severe Opiate use disorder (OUD) are normally averse to talking any type of mu antagonist because it will readily precipitate moderate to severe withdrawal symptoms if the patient has not already undergone a formal opiate detoxification process for at least 5-7 days prior to the administration of a mu antagonist. In this embodiment, the combination therapeutic compounds are designed specifically for harm-reduction in a population that may knowingly or unknowingly expose themselves to the risk of FIMR from F/FAs exposure. Appropriate compound combinations include: PAOU1, PAOU2, PAOU3, PAOU4, PAOU5, PAOU6, PAOU7, PAOU8, and PAOU9.

### Example 6: Methods of Prophylaxis for First Responders

These combination formulas and compounds have been designed to optimize prophylaxis and protection of individuals who are inadvertently exposed to F/FAs and risk of FIRMR/WCS and are trying to optimize their own physiologic function and safety in such an environment. These individuals are unlikely to be habitual or illicit IV opioid users and are therefore at minimal risk for developing any type of opioid related withdrawal symptoms. The use of these compounds are contraindicated in individuals with recent use of opiates or opioids and can be screened by urine sample with a urine drug screen—UDS to look for the presence or absence of opiate/opioid metabolites prior to prophylaxis administration. In the event that someone may have recently received opiates as part of medical treatment, a UDS or naloxone challenge test (for instance, a short-acting mu antagonist like naloxone for this test because it would only last 5-10 minutes of withdrawal symptoms versus hours and hours of severe withdrawal) can be given prior to administration of the prophylaxis agent (e.g. Naloxone 0.8 mg is injected SC into the forearm and the patient is monitored for 30″ for physiologic signs of withdrawal such as elevated HR or BP and other symptoms of precipitated withdrawal such as dilated pupils, sweating and abdominal pain or cramping). Appropriate compound combinations include: PFR1, PFR2, PFR3, and PFR4

### Example 7: Assessment of α-1 Adrenergic Antagonists and Anticholinergic Agents

This example describes methods for assessment of α-1 adrenergic antagonists and anticholinergic agents (i.e., atropine, glycopyrrolate and cholinergic agents (muscarinic and nicotinic agonists), in their efficacy in preventing or reversing fentanyl induced muscular rigidity (FIMR), FIRMR and laryngospasm. Also described are methods for assessment of adjunctive reversal agents for prophylaxis and reversal of FIMR/FIRMR/WCS in an animal model.

WCS Animal Model: This experimental series will use an innovative animal (rat) model of WCS for validation of underlying physiologic mechanisms of WCS, specifically upper airway effects of F/FAs and FIRMR in order to test lead compounds for treatment of symptoms of toxic F/FA exposure or overdose. Hypothesis 1: A new animal model with face validity for human VCC and WCS can be used to identify and/or characterize lead compounds for F/FA toxicity.

Rationale and Background: The key feature of F/FA-induced WCS in humans is the rapid onset of respiratory failure with laryngospasm/vocal cord closure (VCC) and loss of pulmonary compliance and FIRMR/WCS (Scamman, Anesth Analg 62:332-334, 1983) and appears to be the most likely cause of death from F/FA overdose (Somerville et al., MMWR 66:382-386. 2017). In fact, individuals with tracheostomies that bypass the vocal cords (VC), tolerate high dose F/FA without developing WCS, demonstrating that VCC is the key feature of WCS severity (Scamman, Anesth Analg 62:332-334, 1983). VCC was documented in 28 of 30 human adult subjects using fiber optic visualization of the larynx with high dose F/FA (Bennet et al., Anesthesiology 8(5):1070-1074, 1997). These studies indicate WCS from F/FA exposure has a complex etiology, and that effective treatment development requires an innovative animal model for evaluation of potential therapeutic compounds, as previous animal models have not evaluated laryngeal and respiratory muscle function directly. The inventor proposes a novel, experimental animal model for WCS to better replicate human WCS. This innovative model facilitates quantitative microscopic video monitoring of the laryngeal aperture as a measure of VCC and upper airway changes, while using an anesthetic technique and upright positioning that will optimize spontaneous respiration and minimally suppress airway reflexes. Most of the previous work with animal models of WCS occurred prior to the definitive human study by demonstrating the key involvement of VCs in humans with WCS induced by F/FA. Prior models have either bypassed VC with endotracheal intubation or tracheostomy or left the VCs unobserved, therefore the direct effects of previous therapies on VC function and upper airway mechanical failure are unknown. There has been no definitive work on alpha 1 adrenoceptor or subtype antagonists or cholinergic agents in a WCS model and our preliminary data are the first effort to demonstrate the potential role of alpha 1 adrenoceptor and cholinergic receptor subtypes in symptoms of F/FA toxic exposure.

Experimental Design: Development of a rat airway monitoring model for lead compound identification for F/FA exposure is adapted from Yang et al., Anesthesiology, 77(1): 153-61, 1992; and Rackham, Neuropharmacology, 19(9):855-9, 1980. On the day of the procedure rats (male and female Sprague Dawley, 250-300 gm) will be administered ketamine (e.g. 10 mg/kg, i.p.). After onset of anesthesia, animals will be immobilized on a rodent intubating stand. An oral retractor will be placed. Pulse oximetry, plethysmography, and end-tidal CO₂ monitoring will be used to characterize pulmonary function, chest excursion, and gas exchange, respectively. Cardiac function will be monitored with subcutaneous electrocardiography. The femoral artery and vein will be cannulated for blood samples, arterial pressure monitoring, and drug administration. Rectal temp will be kept at 37+/−0.5° C. using a heat lamp and temperature controller. An IV infusion of ketamine will be (50-500 mcg/kg/min by pump) will maintain sedation, analgesia and spontaneous respiration. A digital video microscope will be positioned for continuous visualization of the larynx.

Electromyographic (EMG) signal will be acquired as described and adapted from previous work (Weinger et al., Brain Res, 669(1):10-8, 1995; Rackham, Neuropharmacology, 19(9): p. 855-9, 1980; Benthuysen et al., Anesthesiology, 64(4):440-6, 1986; Yadav et al., Int J Toxicol, 37(1):28-37, 2018). Briefly, monopolar recording electrodes will be percutaneously inserted into the left gastrocnemius muscle and lateral abdominal wall and a ground electrode will be placed in the right hindlimb. As previously described, high dose F/FAs have a stereotypical EMG presentation of sustained isometric contraction from ongoing muscle fiber activity (Weinger et al., Brain Res, 669(1):10-8, 1995). The raw EMG signal will be amplified, filtered and recorded for 5 minutes before, and at least 30 minutes after administration of the test substance. Total EMG activity from each site will be averaged every 5 minutes for calculating the ED100 and 95% confidence limits of each F/FA tested. Regression analysis will be used to calculate ED50 and 95% confidence limits for reduction of rigidity from lead compounds tested.

Calculation of Dose response curve/ED 100 for F/FA VCC and WCS. Rats will be randomized into experimental groups and we will estimate ED100 for VCC and WCS for each F/FA. F/FAs will be administered by infusion pump 10 mcg/kg/min or a comparable dose rate based on the potency of the analogue compared to fentanyl, from MOR binding studies. Carfentanil is 100× the relative potency so will be administered at 0.1 mcg/kg/min) until the animal demonstrates VCC (significant closure of glottis structures or appears to have airway obstruction) and/or WCS. Each analogue will be administered until 4 animals have consecutively demonstrated VCC and WCS. In the event that an analogue does not produce VCC in a test subject at a proportional dose to fentanyl, we will increase the baseline dose by 25% until a consistent effect of VCC is seen in 3 test subjects. Time to effect and dose will be recorded for VCC/WCS and used to plot a dose response curve for each. Vital signs will be noted at the time of VCC and each analogue group will be monitored for 30 min for return of spontaneous respiration. If no return at the end of this time, the animal will receive a final bolus of both ketamine 200 mg/kg and fentanyl 20 mg/kg for euthanasia as adapted from previous work (Yadav et al., Int J Toxicol, 37(1):28-37, 2018).

Use of selective alpha 1 adrenergic receptor agonists/antagonists to demonstrate WCS in vivo: Alpha 1 adrenergic subtype antagonists will be used to isolate each receptor subtype as previously described by Sohn et al., Anesthesiology, 103(2): 327-34, 2005. Alpha 1 subtypes (2 of 3 alpha 1 subtypes) will be antagonized and the third subtype will be agonized with NE and EPI until all combinations have been tested (Sohn et al., 2005). 29. Use of specific alpha 1 subtype antagonists in vivo to systematically and selectively isolate and block each subtype (1A: 5-Methylurapidil, 11B: chloroethylclonidine, 1D: BMY 7378)29 and each combination of subtype (1A+1B, 1A+1D, 1B+1D). A range of physiologic NE doses will be administered to each group with isolated receptor subtypes}} EMG will be used, and direct view microscopy of the VCs will gauge the occurrence of acute airway closure and/or WCS of respiratory muscles (>50% closure of laryngeal aperture with O₂ sat<94% and end tidal CO₂>50 mmHg, EMG value sustained contraction>50% of baseline for 5 minutes).

Preclinical drug characterizations and lead molecule identification in animal model of WCS. A series of alpha 1 adrenoceptor antagonists, opioid receptor antagonists/agonists cholinergic agents as described in formulations noted above, will be administered in a dose range and at different time points after F/FA IV administration to establish which agents may be effective in the reversal of WCS or components of WCS (chest wall/diaphragm rigidity (FIRMR) and VCC, cardiovascular compromise) and may have clinical utility for F/FA toxic exposure and/or overdose and or combined with F/FAs for analgesia with reduced side effect profile. Each reversal agent will be administered at several time points (e.g. given at Time 0, T+1 - - - T+10 etc.) following each individual F/FA administration to identify lead compounds that can reverse or antagonize WCS.

Proposed Drugs and doses tested: 1) Non-selective antagonist: prazosin, 1-500 mcg/kg or 50, 100, 250 mcg/kg; 2) terazosin 10-200 mcg/kg or 70, 200 mcg/kg; 3) selective antagonist: tamsulosin 1-10 mcg/kg or 5, 10 mcg/kg; 3) Alpha 2 agonist: clonidine, 1-200 mcg/kg or 35, 175 mcg/kg; MOR antagonists: 1) naloxone 0.01-1 mg/kg or 0.1, 0.5, 1 mg/kg; 2) nalmafene 1-100 mcg/kg or 25, 50, 100 mcg/kg; 3) naltrexone 0.1-1.0 mg/kg or 0.35, 0.7, 1.0 mg/kg; Cholinergic agents: 1) Atropine 0.05-1 mcg/kg 2) Glycopyrrolate mcg/kg 3) pilocarpine 0.015-0.05 mcg/kg and other muscarinic agonists 4) Nicotine and/or other nicotinic agonists (0.1-2 mg/kg). Combinations will be determined based on efficacies in the rat model.

Timing: Drugs will be administered at 3, 6, and 9 minutes after F/FA administration. These time points may be expanded, for instance to include T minus 60, T minus 45, T minus 30, T−15 T−10, and so forth. Simultaneous administration of F/FAs in various combinations with the agents listed herein will be used to assess their potential for the development of opioid analgesic agents (e.g. F/FAs) with modified side effect profiles (e.g. respiratory depression, laryngospasm, FIRMR, WCS, addiction etc.) and thereby enhance or increase the safety margin and potential for extended ranges of analgesia.

Lead compounds will be defined as: Reversal of VCC/laryngeal aperture by 50% or more, O₂ saturation is greater than or equal to 94% and end tidal CO₂ is less than 50 mmHg, and reversal of rigidity as measured by EMG is 50% or more from F/FA effects, and modified from Bennett et al., Anesthesiology, 87(5): 1070-4, 1987; and Weinger et al., Brain Res, 669(1): 10-8, 1995.

Data Analysis: We will plot dose response curves and timing of response for each analogue. Data from the experiments will be analyzed individually. For each drug, a two-way ANOVA will be performed to evaluate the effect of drug dose (between-subject factor) on EMG, VCC, WCS and blood pressure over time (within-subject factor). This will be followed by Newman-Keuls a posteriori tests to assess dose effects at individual time points as well as differences in EMG activity over time within each dose group (Willette et al., J Pharmacol Methods, 17(1):15-25, 1987; Willette et al., Eur J Pharmacol, 91(2-3):181-8, 1983). Data will be expressed as mean+S.E.M., a p<0.05 will be considered to be statistically significant as adapted from Weinger et al., Brain Res, 669(1): 10-8, 1995.

Expected Results: The objective of this study is to identify drugs that can be used in combination to either reverse or prophylax against WCS in situations of overdose and/or toxic exposure and for the development of F/FAs with limited WCS side effects risk. It is believed that VCC with high dose F/FAs will be a prominent feature of the clinical presentation in the animal model, as seen in humans. Rats and humans have similar anatomic innervation of VCs by the vagus nerve from the medulla and the receptor distributions of alpha-1 adrenergic receptors, cholinergic and opioid receptors in the CNS indicating that this model will predict effective therapeutic agents that can be successfully trialed in humans for the treatment of F/FA induced WCS and respiratory depression. Data obtained from the herein-described experiments will provide dose response curves with the drugs tested that will predict effective/therapeutic drug dosing ranges and drug combinations to prevent FIRMR/laryngospasm (WCS) in these animals and similarly in humans. This will provide a model for future analogue testing and targeted drug development.

Some drug combinations are expected to be more or less effective in a particular dosing vehicle. Thus, different delivery modes, escalating dose regimens, and multiple/concurrent modes of delivery will be explored in this model to increase efficacy (e.g. inhaler, nebulizer, ophthalmic (IOC), PO, sublingual or nasal delivery, IM, IO, IV etc.). These studies will provide lead molecules for treating and/or preventing WCS (FIRMR and laryngospasm) and respiratory depression resulting from F/FA overdose or toxic exposure and/or F/FAs combined with morphine derived alkaloids (heroin). This will also provide a model for the development of F/FAs with modified side effect profiles (e.g. FIRMR, laryngospasm, respiratory depression) that can be used safely for analgesia at low and high doses with minimal side effects. Simultaneous administration of F/FAs in various combinations with the agents listed herein will be used to assess their potential for the development of opioid analgesic agents (e.g. F/FAs) with modified side effect profiles (e.g. respiratory depression, laryngospasm, FIRMR, WCS, addiction etc.).

### Example 8: Experiments & Clinical Trials

This Example provides brief descriptions of studies that will provide additional data related to the herein described technology.

Human Receptor Cloning Studies for Ki and Affinity Binding Studies of F/FAs and Assorted Agents:

The objective of the studies in this example are to characterize F/FA and alpha 1 adrenoceptor antagonists and cholinergic agents interactions with recombinant human alpha 1 adrenoceptor and cholinergic receptor subtypes, and effects on receptor function, to identify lead compounds for treating VCC/laryngospasm and WCS. Hypothesis: In addition to opioid receptors, F/FAs interact with specific alpha 1 adrenoceptor and cholinergic receptor subtypes in a pattern that facilitates WCS in humans. Our mechanistic model can be used to identify possible underlying mechanisms of WCS, identify lead molecules that competitively block or inhibit binding of F/FAs to these receptors as a treatment against F/FA toxic exposure and/or overdose and will also facilitate development of F/FAs with modified side effect profiles for safer analgesia with high dose F/FAs.

To characterize how fentanyl, sufentanil, alfentanil, carfentanil, and several other F/FA analogues (e.g. acetyl-fentanyl, ohm-fentanyl) and morphine (e.g. heroin) differ in receptor interactions, we will compare their Ki values using cells transfected with human recombinant alpha 1 adrenoceptor subtypes, human recombinant cholinergic receptor subtypes and will also generate IC₅₀/EC₅₀ values in assays of receptor function. After transfection with specific recombinant human cDNAs, it will be confirmed that the transfected cells express the receptor subtypes at levels that allow for their use in medium- and high-throughput screening of drugs, with Bₘₐₓₑₛ in the pmol range.

Expected results: It is expected that fentanyl, but not morphine, will block radioligand binding to the alpha 1 adrenoceptor 1A and 1B subtypes with weaker binding at the 1D receptor. Norepinephrine (NE) will bind these subtypes with different affinities, and have the most potent effects at the 1D receptor subtype, compared to its affinities at the 1A and 1B subtypes, suggesting that NE and the 1D receptor subtype, play a key role in the underlying mechanism for F/FA toxicity/WCS laryngospasm/FIRMR and may be useful in designing treatments for F/FA toxicity that block these effects. Fentanyl and at least some of the other F/FAs to be tested will bind with varying affinity at the muscarinic and possibly nicotinic subtypes, and that selective binding particularly at muscarinic subtypes (M1-M5) will increase the selective binding of ACH at the subtypes unoccupied or weakly bound (less affinity than ACH) by fentanyl. We anticipate that the subtype binding pattern of fentanyl and ACH at the muscarinic receptors subtypes will support our hypothesis (C1.), that this binding pattern facilitates vocal cord closure and laryngospasm in high dose F/FA overdose and/or toxic exposure. Conversely, we anticipate that agents that block this binding pattern will be potentially useful as therapeutic agents in treating WCS and in the development of safer F/FA based analgesics that have limited side effects (e.g. decreased laryngospasm, FIRMR and respiratory depression) for safer analgesia with high dose F/FAs.

To determine if fentanyl is an agonist or antagonist at alpha and cholinergic receptors, ELISAs will be used to examine receptor-mediated inositol-1 phosphate (IP-1) accumulation. Specific alpha and cholinergic (muscarinic) receptor subtypes are believed to play a role in WCS, which demonstrates important differences between morphine, naloxone and F/FA-receptor interactions. It is proposed that NE will interact with the 1D receptor subtype while the 1A and 1B subtypes are blocked by fentanyl; this is believed to play a major role in WCS and sudden cardiac events. The 1D receptor is the predominant subtype expressed in coronary arteries, and 1D agonism causes vasoconstriction and compromised cardiac function. Thus, pharmacotherapies could include alpha 1 adrenergic receptor ligands directly, or indirectly via interaction with alpha 2 receptors that alter NE release.

Similar transfection studies with human cholinergic receptors (e.g. muscarinic and nicotinic receptors) will characterize ACh, fentanyl and F/FAs subtype selectivity binding. The resulting data will indicate that fentanyl and other F/FAs allow for a binding pattern model of ACh to muscarinic receptors that could facilitate changes in motor control and vocal cord patency (laryngospasm) in vivo.

Experimental Design: Binding studies and receptor function. We will examine: A) the Ki values of F/FAs, tamsulosin, terazosin, prazosin, droperidol, naloxone and morphine (heroin) on [³H]prazosin binding to each alpha receptor subtype, and determine the affinity of antagonists and F/FAs at each alpha adrenoceptor subtype. B) F/FAs that are potent (Ki≤1 μM) at displacing [³H]prazosin from any alpha adrenoceptor subtype will be tritiated to determine (conversely) if alpha 1 adrenoceptor subtype antagonists can displace those [³H]F/FAs from alpha receptors directly. C) the Ki values of F/FAs, acetylcholine (ACh), nicotine, atropine, glycopyrrolate, droperidol and pilocarpine on [³H]atropine and ³H]pilocarpine and [³H]acetylcholine, binding to each muscarinic and nicotinic cholinergic receptor subtype and determine the affinity of antagonists and F/FAs at each cholinergic subtype. D) F/FAs that are potent (Ki≤1 μM) at displacing [³H]acetylcholine from any muscarinic or nicotinic subtype will be tritiated to determine (conversely) at what concentration the muscarinic or nicotinic subtype antagonists/agonists can displace those [³H]F/FAs from muscarinic and nicotinic receptors directly.

My lab is currently using the following: [³H]-fentanyl, -alfentanil, -carfentanil, and -sufentanil for our work and can tritiate F/FAs as needed, e.g., acetylfentanyl. E) We will examine effects of agonists, antagonists and F/FAs in IP-1 assays of function.

Radioligand binding methods: [³H]prazosin, [³H]tamsulosin, [³H]terazosin, [³H]atropine, [³H]droperidol, [³H]glycopyrrolate, [³H]pilocarpine (or a comparable M3 agonist) and [³H]F/FAs. Radioligand binding experiments will be conducted using the previously described methods (Eshleman et al., Biochem Pharmacol, 85(12): p. 1803-15, 2013; Gatch et al., J Pharmacol Exp Ther, 338(1):280-9, 2011; Shi et al., PLoS One, 11(3):e0152581, 2016) with validated receptor characterization panels. Briefly: To characterize drug interactions with the alpha 1 adrenoceptor 1A, 1B, and 1D subtypes, muscarinic M1-M5 and nicotinic receptors (nicotinic acetylcholine receptor α4, α7, β2 subunits and α4β2), HEK-293 cells are transfected using polyethylenimine (PEI) as previously described [36]. When confluent, the media is removed, cells are rinsed with phosphate-buffered saline (PBS), scraped into PBS, and prepared for binding assays, adapted from published methods (Shi et al., PLoS One, 11(3):e0152581, 2016). Assays are performed in duplicate in a 96-well plate. Serial dilutions of test compounds are made using the Biomek 4000 robotics system. Membranes are preincubated with drugs (9 concentrations, 10⁻¹⁰ to 10⁻⁵ for the first experiment and then adjusted so that at least 6 concentrations are on the slope of the curve) for 10 min prior to addition of [³H]prazosin etc. for alpha receptors, or [³H]atropine ³H]pilocarpine etc. for muscarinic receptors and [³H]nicotine etc. for nicotinic receptors (as per their respective receptors) (1-2 nM final conc., 80 Ci/mmol, Perkin Elmer) in a final volume of 250 μl. Nonspecific binding is defined with 10 μM phentolamine for alpha receptors, and ACH, atropine and nicotine for their respective receptors. The reaction is incubated for 45 min at 25° C. and terminated by filtration over 0.05% PEI-soaked “A” filtermats using cold Tris buffer (50 mM, pH 7.4) with a 6 sec wash. Validation compounds include acetylcholine, atropine, doxapram, droperidol, epinephrine, glycopyrrolate, heroin, morphine, norepinephrine, naloxone, naltrexone, nalmafene, nicotine, pilocarpine, phenylephrine, prazosin, phentolamine, tamsulosin, terazosin, (1A) 5-Methylurapidil, (1B) chloroethylclonidine, and (1D) BMY 7378 for alpha adrenergic receptors and acetylcholine, muscarine and nicotine for cholinergic (e.g. muscarinic and nicotinic) receptors, respectively. The filters are spotted with scintillation cocktail, and counted on a Perkin Elmer microbetaplate counter. For [³H]F/FA binding, very similar methods are used, except that non-specific binding is determined with fentanyl (5 μM). (Naloxone is a poor indicator of nonspecific binding).

Inositol-1-phosphate (IP-1) formation: Previous studies indicate that fentanyl is not an agonist at alpha 1 receptor subtypes, but its effects at nicotinic receptors and muscarinic receptors remain unknown. However, substituents on the F/FA backbone might confer agonist activity so it is possible that some F/FAs might stimulate alpha receptors, muscarinic and/or nicotinic receptors in assays of function (Sohn et al., Anesthesiology 103: 327-334, 2005). HEK-Adr1A, HEK-Adr1B or HEK-Adr1D cells and the IP-One1 Gq ELISA kit are used. The methods are adapted from previous publications of IP-1 assay methods (Yang et al., Anesthesiology, 77(1):153-161, 1992). Agonists are normalized to the maximal stimulation by NE and antagonists are tested in the presence of 100 nM NE and normalized to the inhibition by 100 nM tamsulosin. In the case of muscarinic and nicotinic receptors we will use HEK transfected cells with each respective receptor subtype and agonists will be normalized to the maximal stimulation by acetylcholine 100 nM ACh and normalized to the inhibition of atropine for muscarinics.

Data analysis. Radioligand competition binding data are normalized to binding in the absence of a competitive (naloxone, fentanyl, etc.) drug. Three or more independent experiments are conducted with duplicate determinations. GraphPAD Prism is used to analyze the subsequent data, with IC₅₀ values converted to Kᵢ values (Eshleman et al., Biochem Pharmacol, 85(12): p. 1803-15, 2013). Differences are assessed by one way ANOVA using the log of the Kᵢ values. Tukey's multiple comparison test is used to compare potencies and efficacies. For functional assays, GraphPAD Prism is used to calculate either EC₅₀ (agonists) or IC₅₀ (antagonists) values using data expressed as % NE-stimulation for IP-1 formation. For functional assays, one way ANOVA is used to assess differences in efficacies using normalized maximal stimulation, and differences in potencies using the logarithms of the EC₅₀ values for test compounds. Tukey's multiple comparison test is used to compare test compounds with significance set at p<0.05.

Expected Results: Data will indicate that fentanyl but not morphine displaces radioligand from specific alpha 1 adrenoceptor subtypes, and that fentanyl but not morphine is a functional antagonist at alpha adrenergic specific subtypes. Further, data will indicate that fentanyl but not morphine will displace radioligands at alpha adrenergic receptor subtypes in a selective distribution that will facilitate NE binding at these alpha adrenergic receptor subtypes in a manner that will allow for and/or support the underlying mechanisms of WCS as described above. It is also expected that data will indicate that fentanyl but not morphine will displace radioligands at muscarinic and possibly nicotinic receptors in a selective distribution that will facilitate ACh binding at cholinergic receptors in a manner that will allow for and/or support the underlying mechanisms of WCS as described above.

A-1 Fentanyl Reversal Animal Protocol

Once the “affinity binding” and “animal studies” have established lead compounds, the compounds will be tested for safety in animals and an FDA IND application will be filed for testing in human subjects. Two sets of human clinical trials are described.

Trial A—Operating Room Trial:

Trial A: (Anesthesia model) Rationale: The rapid onset of accelerated respiratory failure/depression seen with F/FAs was first witnessed by anesthesiologists administering large doses of F/FAs to human patients for surgical procedures. Anesthesiologists subsequently became the clinical experts for managing the rapid onset of acute respiratory and airway complications induced by F/FAs. Subsequent human studies using video laryngoscopy of vocal cords definitively demonstrated that large doses of F/FAs result in rapid vocal cord closure (90-120 seconds) prior to the development of chest wall rigidity and respiratory failure/depression (Grell et al., Anesth Analg 49(4):523-532, 1970; Streisand et al., Anesthesiology 78(4):629-634, 1993; Bennett et al., Anesthesiology 87(5):1070-1074, 1997; Scamman, Anesth Analg 62:332-334, 1983). In the uncontrolled setting of drug overdose, F/FA induced vocal cord closure can result in accelerated respiratory failure, hypoxia and death in less than 3 minutes as noted by autopsy data of F/FA overdose decedents. Considering the unprecedented urgency of the opioid crisis methodology must be used that can rapidly identify therapeutic agents.

To optimize the feasibility of drug efficacy in the field, testing of reversal and prophylaxis drugs in human subjects should first be performed in the controlled setting of an OR or ICU to avoid the multiple confounding variables that would occur in an EMS/field setting of first response to opioid overdose. In the setting of first response to opioid overdose, information is limited. The exact dose and drug characteristics are rarely known and serologic testing and drug analysis are currently not feasible or available prior to emergency treatment. Therefore, the most specific methodology for directly and safely demonstrating the efficacy of drugs that prevent acute VCC and accelerated respiratory failure from F/FAs in human subjects, would involve an OR based study where these drugs (lead compounds could be administered by anesthesiologists (the clinical experts in airway management and F/FA administration), to healthy subjects in a clinically controlled environment, where the efficacy of these lead compounds can be accurately evaluated. This invention teaches a novel and safe methodology for evaluating drug treatment for WCS in humans. Similar clinical studies in healthy adult subjects have consistently demonstrated the viability and safety in performance of such trials in an OR based setting and the study proposed in Methods below is an adaptation of these previous studies with the added novel safety parameters and advantages of administering pharmacologic interventions to a test subject with a secured airway.

Trial A: Methods: Human “Trial A” Operating Room Trial

After full review and approval by the IRB committee of the institution(s) sponsoring the trial, HUMAN “Trial A” will test the efficacy of the prophylaxis agents and reversal agents as identified by the animal trial as previously described and approved for clinical trial after FDA IND review and completion of all required safety testing. The trial will consist of 5 protocol arms to evaluate the proposed lead compounds as either reversal or prophylaxis drugs compared against the mu antagonist—naloxone as a single agent. Consented ASA class I and II volunteer study subjects scheduled for non-emergent, elective surgery that requires general anesthesia and intubation. Approximately 50 subjects will be randomized into 1 of the 6 protocol arms (1. naloxone 2. naloxone+alpha 1 agent/s 3. naloxone+cholinergic agent/s 4. cholinergic agent/s+alpha 1 agent/s 5. naloxone+cholinergic agent/s+alpha 1 agent/s 6. F/FA cholinergic agent/s+alpha 1 agent/s+respiratory accelerant). Each of the 60 subjects will receive a high dose of F/FA after the airway has been secured and will receive one of these compounds at a time interval that is either prior to the F/FA dose or at a designated time point after administration. In the case of the simultaneous administration of an F/FA with cholinergic agent/s+alpha 1 agent/s+respiratory accelerant, this will be designated as the “analgesia arm” that will be testing this combination as an F/FA analgesic with a modified side effect profile (e.g. decreased FIRMR, laryngospasm and/or respiratory depression) that allows for high F/FA analgesic doses with minimized side effects.

Reversal Arm: The reversal treatment arm of the study will employ an established opioid reversal dose protocol administered after the F/FA dose and consisting of the current standard of care mu antagonist naloxone given as a single agent or as a compound designated as “naloxone+” consisting of naloxone and an agent specifically designed to reverse WCS that includes: 1) a Mu receptor antagonist and 2) an alpha-1 adrenoceptor antagonist (A1ARA) or a combination of subtype selective and non-selective A1ARAs, or 3) a cholinergic agent (anticholinergic e.g. atropine or muscarinic agonist e.g. pilocarpine or nicotinic agonist e.g. nicotine) or 4) cholinergic agent/s+alpha 1 agent/s or the single agent droperidol or 5). naloxone+cholinergic agent/s+alpha 1 agent/s for the reversal of WCS in patients administered IV fentanyl by infusion in the setting of an operating room with a board certified Anesthesiologist and surgical staff present to manage and care for the study participants.

Prophylaxis Arm: A further randomization will occur in that some of the participants will be given a single dose of an oral “prophylaxis agent” 1 hour prior to the procedure consisting of 1 or more of 4 agents: 1) a long acting mu antagonist such as naltrexone or nalmefene as a single agent or; 2) Mu antagonist in combination with an A1ARA or; 3) a combination of selective and non-selective A1ARAs; 4) a cholinergic agent (anticholinergic e.g. atropine or muscarinic agonist e.g. pilocarpine or nicotinic agonist e.g. nicotine) 5) droperidol. If the patient develops rigidity and evidence of laryngeal nerve activity by EMG or evidence of increased tracheal pressure, the infusion will be stopped and the immediate reversal agent naloxone or naloxone+ will also be given.

Analgesia Arm: Simultaneous administration of F/FAs in various combinations with the agents listed herein (e.g. simultaneous administration of F/FA with alpha, anticholinergic, and resp accelerant, nicotine etc.) will be used to assess their potential for the development of opioid analgesic agents (e.g. F/FAs) with modified side effect profiles (e.g. respiratory depression, laryngospasm, FIRMR, WCS, addiction etc.) and thereby enhance or increase the safety margin and potential for extended ranges of analgesia.

Trial A Methods: This human trial will consist of approximately 40 ASA 1-3, Class I-II airway, adult volunteer subjects (male and female subjects age range matched for community overdose) in an operating room with all available anesthesia, non-invasive monitoring, and IV access. All subjects will be NPO for 8 hrs and will have a chart review, interview and history and physical prior to the procedure and study. Prior to this day of interview, each study subject will have also had a pre-screening interview as part of the initial screening and eligibility review to answer questions and document legal consent to participate in the study.

The study subjects will be attended by board certified/qualified anesthesiologists and surgical staff/nursing staff. Each group (e.g. control groups and experimental groups) will be pre-medicated with IV midazolam (1 mg), brought into the OR, transferred to the OR bed with all monitors placed (EKG, blood pressure, pulse oximetry), pre-oxygenated with a face mask attached to anesthesia machine and circuit. 100% oxygen will be administered by the circuit at a rate of 15 LPM for 5 minutes prior to any medication infusion.

The study will be performed using recurrent laryngeal nerve monitoring (Medtronic NIM Tri-vantage EMG ETT) where the subject is intubated after receiving fentanyl (1 mcg/kg), propofol (2-3 mg/kg) and a short acting muscle paralytic (e.g. succinylcholine at 1 mg/kg) to secure the airway and GA maintained with sevoflurane and O₂. N₂0 will be avoided in these cases to eliminate the risk of exaggerated rigidity responses due to its interaction with fentanyl. An EMG ETT will be placed with optimal positioning, and signal tested and baseline recording made prior to the administration of F/FAs. The pilot balloon cuff will be inflated with water instead of air to prevent any pressure change from gas diffusion or absorption into the balloon cuff. An electronic pressure monitor with continuous pressure measurement capabilities will be used to measure the presence/absence of pressure change (e.g. cm H₂0 pressure) in the trachea with administration of F/FA during the trial period. GA will be maintained until the peripheral neurostimulator registers “train of four” (TOF 4/4) with minimal micro voltage setting (e.g. confirmation that the neuromuscular blockade has worn off) and the tracheal pressure and EMG are stable for 10″. After all baseline vitals have been taken over this 10 minute period, the patient will receive an infusion of 3 mcg/kg/min of fentanyl with total dose to 30 mcg/kg, or a comparable (equipotent) doses of either sufentanil, alfentanil or remifentanil.

Tracheal pressures and EMG activity will be monitored with readings noted at (“Time-0”) or just prior to F/FA infusion and then every 60 seconds/in one minute intervals after the infusion begins. Ideally, each parameter will be measured separately by an individual/monitor and the anesthesiologist is actively monitoring the patient and maintaining a GA to within 10-25% of baseline BP and pulse rate. We will take consecutive measurements of the following parameters at 1 minute intervals until the drug (F/FA) infusion is complete or until the subject develops/meets criteria for study intervention or either treatment outside of the study parameters: 1) Tracheal pressure (and Peak AW pressures) 2) EMG activity of laryngeal nerves (recurrent laryngeal/superior laryngeal nerve branches) 3) Qualitative rigidity rating scale (assess presence/absence of rigidity in 4 defined muscle groups) 4) Vital signs (BP, HR, EKG, O₂sat, ETCO₂)

The parameters are defined as: 1) Tracheal pressure (and Peak AW pressures); 2) EMG activity of laryngeal nerves (recurrent laryngeal/superior laryngeal nerve branches) 3) Qualitative rigidity rating scale (assess presence/absence of rigidity in 4 defined muscle groups) 4) Vital signs (BP, HR, EKG, O2sat, ETCO₂) The time and dose will be registered for significant EMG activity defined from previous studies and adapted from Weinger et al. 1989) as (>50% of baseline), significant Tracheal pressure (defined as (>20%) and/or qualitative observation of rigidity of skeletal muscle rigidity involving at least 3 muscle groups (1) head/neck, 2) upper extremities, 3) chest wall/abdominal wall and 4) hip flexors/lower extremities) or a sudden onset of peak airway pressures indicating bronchospasm and inadequate ability to maintain ventilation without administration of a muscle paralytic.

Subjects will receive 1-3 treatments of the randomly assigned reversal compound (naloxone or naloxone plus).

Study Protocol intervention prior to completion of total infusion dose: If at any time the patient develops significant muscle rigidity in (1 face/neck or 2 upper extremities, 3 chest wall/abdomen and 4 lower extremities) or demonstrates a significant change in peak pressures (>25% cm H₂O), the reversal drug will be administered as per the subject's prior randomization and/or a second dose of muscle paralytic if there appears to be no improvement or worsening of condition.

Trial A: Data collection/Data Sources: EMR from anesthesia machine of all vital sign and physiologic parameters including, but not limited to BP, HR, EKG, O2sat, ETCO₂, EEG, Bispectral index, pulmonary mechanics, EMG and EMR record of case/anesthetic/EMG from NIM and Tracheal pressure readings.

Trial A: Expected Results: Since both the timing of onset and duration of FIMR have been well documented in previous human trials (Streisand et al., Anesthesiology 78(4):629-634, 1993), any measured difference in the dose response curve or the duration curve of FIMR between control groups and the experimental groups would be attributed to the presence or absence of prophylaxis and the use of an immediate reversal agent as described above. The expected outcome in the experimental group is that FIMR/FIRMR/laryngospasm will either be inhibited completely or the dose response curve will be shifted to the right indicating that a greater level of fentanyl is required in order to induce FIMR/FIRMR/laryngospasm, thus improving the safety margin to the potential exposure of F/FAs. We also anticipate that subjects receiving prophylaxis agents will show a significant inhibition of laryngeal and FIRMR effects from high dose F/FAs tested. We anticipate that if these subjects develop WCS symptoms that they will require lower doses of reversal agents to reverse WCS than non-prophylaxis subjects. We also anticipate that naloxone as a single agent will be ineffective at reversing or significantly inhibiting WCS symptoms in dose ranges that are normally used for treatment of opioid induced respiratory depression. In the event that FIMR/FIRMR/laryngospasm occurs, experimental subjects will be given immediate reversal agents consisting of the naloxone plus one or more of the other agents being tested in the trial. The timing and duration of FIMR/FIRMR/laryngospasm will be measured and compared to the control group and we anticipate that prophylaxis subjects and naloxone plus reversal agent subjects will have a significantly decreased recovery time to normal breathing mechanics with a significant decrease in the duration and/or intensity of WCS symptoms (e.g. FIRMR and laryngospasm).

Human “Trial B” Emergency Room/EMS Paramedic Trial:

Trial B: Trial B (EMS model) Rational: Acute opioid overdose presents as profound respiratory depression (RD) with anoxia that can lead to death. Administration of the mu opioid receptor antagonist naloxone to reverse RD has become the standard of care as part of out-of-hospital management of opioid overdose (Wanger et al., Acad Emerg Med. 5(4):293-9, 1998). However, in addition to RD, high doses of synthetic opioids, specifically fentanyl and fentanyl analogues (F/FA), also cause Wooden Chest Syndrome (WCS), a clinical presentation consisting of rapid vocal cord closure (laryngospasm; VCC) and severe diaphragm and chest wall rigidity that is often fatal without invasive airway management (Grell et al., Anesth Analg 49(4):523-532, 1970; Bennett et al., Anesthesiology 87(5):1070-1074, 1997). Heroin, an opioid that is metabolized to morphine, is far less potent than F/FAs and is not known to cause WCS. The overall objective of the study design is to determine whether naloxone+ administered IV or IM and/or IN to out-of-hospital patients with suspected F/FA opioid overdose, is more effective at returning functional respiratory mechanics (resolution of respiratory depression and WCS) to increase survival rates in F/FA overdose patients over the control treatment-naloxone.

After full review and IRB approval of the study protocol and FDA IND approval of all test compounds, the institution(s) sponsoring the trial, HUMAN “Trial A” will begin recruitment of patients presenting with acute opioid overdose in an EMS field setting where participants will be randomized to receive an opioid reversal dose protocol that may include: 1) a Mu receptor antagonist and 2) an al Adrenergic Receptor Antagonist (A1ARA) and/or a combination of “selective” and “non-selective” A1ARAs and/or 3) a cholinergic agent (anticholinergic e.g. atropine or muscarinic agonist e.g. pilocarpine or nicotinic agonist e.g. nicotine) and/or 4) droperidol, for treatment of opioid overdose in patients that are suspected of or have a clinical presentation indicative of fentanyl or fentanyl analogues related overdose (e.g. rapid loss of consciousness after injection, rapid onset of cyanosis, chest and upper body rigidity, multiple doses of naloxone used and little or no response, needles and tourniquet still found in/on arm, sudden onset of rigidity or “seizure-like” activity after injection etc.). These individuals will be randomized to receive either naloxone (e.g. the current standard of care) or will receive Naloxone+ given as a multi-component reversal agent as described herein, including a Mu antagonist, an al Adrenergic Receptor Antagonist (A1ARA) or a combination of “selective” and “non-selective” A1ARAs and a cholinergic agent (anticholinergic agent e.g. atropine or a muscarinic agonist e.g. pilocarpine or nicotinic agonist e.g. nicotine).

The success of resuscitation will be measured by indicators of reversal such as the return of spontaneous respiration with adequate tidal volumes to sustain O₂ Saturations>94% with room air or 1-4 L supplemental O₂, ease of assisted ventilation, resolution of muscular rigidity, the return of consciousness and responsiveness as gauged by the Glasgow Coma Scale. Blood samples will be drawn and analyzed for the presence of fentanyl or fentanyl analogues and metabolites such as norfentanyl. This data will be blinded and analyzed and compared with medical records of resuscitation to evaluate for statistical evidence of more rapid resuscitation in individuals suspected of F/FAs related overdose arriving in ER for medical treatment and receiving either current standard of care or a “broad spectrum reversal agent”. One of the expected outcomes will be that individuals who are serologically confirmed to have significant serum levels of F/FAs will show a response to treatment with the “broad spectrum reversal agent” designated as Naloxone+ after no response or little response to multiple doses of the single agent naloxone.

Methods: As adapted from prior clinical studies of naloxone in the opioid overdose from Wanger et al., Acad Emerg Med. 5(4):293-9, 1998 and Sabzghabaee et al., Arch Med Sci. 10(2):309-14, 2014, a multi-center, double blind, randomized control/non-placebo, additive trial of approximately 200 out-of-hospital patients with suspected opioid (synthetic and/or non-synthetic) overdose will be conducted in several urban—out of hospital settings of high endemic areas for F/FA overdose. Time intervals will be compared, from arrival of EMS/paramedics at patient's side to time to response of adequate oxygenation and ventilation (defined as respiratory rate>/=10 breaths per minute with pulse oximetry values>/=94%), presence or absence of muscle rigidity, resolution or improvement of muscle rigidity, return of normal pulmonary compliance as measured by bag-valve-mask ventilation and duration of assistance, vital signs (blood pressure, heart rate and respiratory rate) and change in level of consciousness (Glasgow Coma Scale and descriptive scale “comatose, obtunded, lethargic or conscious”). EMS providers called to the scene of an opioid overdose, while en route, will randomize the patient to receive a color coded vial for IV injection or a color coded IM injector containing either Naloxone or Naloxone+. Once the color coded IV med vial or IM injector is assigned, the same color code will be administered for the duration of the rescue study protocol. EMS/paramedic staff will follow the most current standards of care regarding resuscitation of opioid reversal and BLS and ACLS protocols and will administer the assigned medications within these parameters. Importantly, although it is not expected that the study protocol will deviate from these care standards, adherence to BLS and ACLS standards will always take precedence over the study protocol. Overdose victims will receive up to 3 doses IV/IM of the assigned drug(s) at 3 minute intervals and will be assessed for adequacy of respiration and oxygenation and presence/absence of muscle rigidity, while airway and hemodynamic management is provided. If the patient remains unresponsive and/or hypoxemic or has persistent muscle rigidity after 3 doses, the study protocol indicates immediate rapid sequence induction and securing airway via endotracheal intubation on transport to the hospital ER. In the event of potential aspiration or other airway complications requiring immediate intubation, airway management will take precedence over the study protocol. After patient has been stabilized, serum samples will be drawn for F/FA and drug analysis. Data will be reviewed and analyzed for statistical significance and efficacy of Naloxone+ in reversal of WCS and RD during opioid overdose with F/FAs and/or MDAs, compared to naloxone.

Halfway through the study period at 12 months (˜24 months total duration and ˜200 participants), color codes for the trial drugs will be crossed over. A preliminary data analysis will be performed at that time and if necessary, the protocol will be modified to either lower or increase the dose of Naloxone+ as long as side effects are minimal and the therapeutic efficacy has the potential of improvement with a dose adjustment.

Trial B: Participant recruitment: After IRB approval of the study protocol and FDA IND approval of the test compound/s, patients will be selected/recruited to the study based on the need for life-threatening and emergent treatment for opioid overdose and all IRB criteria. All patients will be treated with the current standard of care for opioid reversal, the mu opioid receptor antagonist, naloxone. Patients may be randomized to receive the additive experimental treatment for WCS. The dose of the additive drug will be in a range and/or combination that has been demonstrated to have a minimal side effect profile in adult humans as per existing and IND human safety study data.

Trial B: Population and setting: The study trial will involve adult patients 18 or older requiring EMS services for a suspected or reported opioid drug overdose and will be based in large urban areas where F/FAs represent a significant proportion (>60%) of all opioid or drug overdoses (e.g. Boston, Miami, Cincinnati, Buffalo).

Trial B: AIM 3 Data collection/Data Sources: In addition to the standardized forms used by paramedic staff for documentation of emergency medical management, data collection for the study to track AIM3 primary and secondary will be performed by paramedic/EMS staff via a standardized series of data management forms designed for visual clarity and binary “yes” or “no” answer format to record data specific to F/FA overdose. Administration times for drugs will be preceded and followed by specific and systematic assessments of vital signs and quantitative and qualitative clinical measures defined below in Study Measures. The data chart will be organized in groups, color coded for each dose administered with an assessment section for each dose, in a flow chart that follows the physiologic course of opioid overdose reversal and/or emergency resuscitation. A side column will be present on the right side of the data sheet to note if ACLS or BLS is being performed at that time or for that assessment. Chart information on demographics and any known or preexisting health history will be noted by paramedic staff after the resuscitation is complete and/or patient care has been transferred to other medical providers or hospital/ER staff. We will obtain client name; date of birth; hospital record number, Medicaid number (if applicable); relevant medical history; primary, secondary and tertiary substance use problem (e.g. heroin, other opiates, fentanyl and other synthetic opioids, alcohol, cocaine, methamphetamine, cannabis etc.); age of first use, frequency of use, route of administration, and awareness of F/FA if present in serum drug screen. All records and data will be stored in a HIPAA compliant fashion. An extensive data encryption plan will be reviewed and approved by IRB and IT committees of participating hospitals or EMS service units prior to implementation of the study or the collection of patient data.

Trial B: Study Measures: The physical signs and symptoms associated with F/FA (WCS) and morphine derived alkaloids (respiratory depression—RD) will be measured. Specifically, time intervals will be compared from arrival of EMS/paramedics at patient's side to development of/time to response of adequate oxygenation and ventilation (respiratory rate>/=10 breaths per minute with pulse oximetry values>/=94%), presence or absence of muscle rigidity, resolution or improvement of muscle rigidity, if present, normal pulmonary compliance as measured by bag-valve-mask ventilation and duration of assistance, vital signs (blood pressure, heart rate and respiratory rate) and change in level of consciousness (Glasgow Coma Scale and descriptive scale). Time to return of spontaneous respiration and time to return of adequate respiration will be noted as described above. If muscle rigidity is present, the number of muscle groups involved (0—no rigidity, 1—jaw, neck, 2—shoulders, upper extremities, 3—chest wall, abdomen, 4—lower extremities) will be noted as per the grading system. If the EMS team is providing assisted ventilation, they will note and grade the ventilation effort required to maintain adequate oxygenation (0—easy or with 1—some effort or 2—difficult or 3—impossible to mask ventilate). Level of consciousness will be evaluated as per the Glasgow Coma Scale where the rating scale is defined as: Insert GCS scale rating system or can say refer to since it is well known.

The patient will receive up to 3 doses of naloxone/naloxone+. Resolution, inhibition or no change in symptoms will be noted 1 minute after each drug administration and prior to the next administration until patient either stabilizes with adequate respiration and oxygenation or requires intubation. The time to return for spontaneous respiration/ventilation and adequate oxygenation and the resolution of muscle rigidity will be analyzed for each group/drug. After 3 doses as per the study protocol, if persistent inadequate respiration and oxygenation is noted or if patient is unstable and requires immediate airway management, the patient will be intubated and the time of induction and intubation will be recorded. Serum samples will be drawn for drug analysis and time of draw noted by EMS/Paramedic team.

Primary Outcome: Time to return to spontaneous respiration/ventilation with adequate oxygenation and resolution of muscle rigidity/FIRMR/laryngospasm/WCS will be measured. Secondary Outcome: Level of consciousness. An overall goal of the study is identification of optimal lead compound efficacy in humans for treating WCS and RD in reversal of F/FA overdose compared to naloxone.

Trial B: Intervention Power Analysis: Data will be analyzed by making comparisons of mean time intervals using an unpaired t-test and verified with nonparametric testing. Power calculations using the results from the control arm of the study will be performed using an a=0.05, power=0.90, A=2.0 minutes (to return of adequate spontaneous ventilation/oxygenation as previously described above) and SD=4.18. Alternatively the “A” variable could be defined as the number of doses required for adequate spontaneous ventilation/oxygenation as a marker of superiority of treatment (e.g. 1-2 doses of N+ with no ETT placed vs. 2-3 doses of N and ETT placed). Based on these preliminary calculations and comparable studies assessing EMS use of naloxone in emergency treatment of opioid overdose, a sample size of 184 (92 per arm) will be required (Wanger et al., Acad Emerg Med. 5(4):293-9, 1998 and Sabzghabaee et al., Arch Med Sci. 10(2):309-14, 2014).

Trial B: Analytical Methods and Sample Size Determinations: Field data forms will be reviewed on a weekly basis to assure appropriate application of suspected overdose protocol, data collection and review of vital signs and clinical charting. Specifically we will review drug combinations used by the patient, assure that vital signs are initially recorded and then every 2-3 minutes after, time of medication doses recorded, dose and route of administration of intervention drug, duration of basic airway intervention and tools used (bag valve mask—BVM/oropharyngeal AW—OPA), total time from drug administration to return of adequate spontaneous ventilation/oxygenation (>10 BPM and O₂ sat>93%)

Primary Outcome: Time to return and number of doses required to return to spontaneous respiration and ventilation with adequate oxygenation and resolution of muscle rigidity/FIRMR/laryngospasm/WCS will be measured. Secondary Outcome: Level of consciousness with return of GCS score to (Decide on GCS score). Overall goal of the study is identification of optimal lead compound efficacy in humans for treating WCS and RD in reversal of F/FA overdose compared to naloxone. As adapted from Wanger et al., Acad Emerg Med. 5(4):293-9, 1998 and Sabzghabaee et al., Arch Med Sci. 10(2):309-14, 2014.

Trial B: Expected Results: “Naloxone (+)” is expected to perform as well if not better than naloxone in antagonizing morphine derived alkaloid induced respiratory depression and will be superior for antagonizing F/FA-induced WCS. Patients receiving naloxone+ will be less likely to require intubation/invasive airway management and multiple doses of medication before primary and secondary outcomes are achieved. It is not anticipated that hemodynamics will be significantly different based on the low dose and selective nature of the alpha 1 adrenoceptor antagonists used for the naloxone+ compound. Overall mortality and morbidity will decrease and the survival rate will be significantly improved for naloxone+ patients who overdosed or were exposed to F/FAs or a combination of F/FAs with morphine derived alkaloids.

As will be understood by one of ordinary skill in the art, each embodiment disclosed herein can comprise, consist essentially of or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As used herein, a material effect would cause a measurable reduction in one or more systems of opioid/opiate usage or overdose (for instance, reduction in chest wall rigidity, increased level of consciousness, return of spontaneous respiration and adequate tidal volumes to maintain O₂ Saturations>94% by pulse oximetry) within one minute to ten minutes following administration of a disclosed combination therapy to a subject (in the case of immediate care/reversal embodiments); or a material effect would prevent or reduce the development of one or more such symptoms upon exposure to an opioid/opiate, in the case of a prophylactic embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A pharmaceutical composition comprising:
a pharmaceutically acceptable carrier; and either
(A) a therapeutically effective amount of a Mu opioid receptor antagonist (MU), and a therapeutically effective amount of a α2-adrenergic receptor agonist (A2ARA); or
(B) a therapeutically effective amount of an α1-adrenergic receptor antagonist, and a therapeutically effective amount of one or more of:
a Mu opioid receptor antagonist (MU), opioid receptor subtype antagonist, opioid receptor subtype agonist, a centrally-acting or peripherally acting respiratory stimulant, a GABA/benzodiazepine receptor complex antagonist, an α1-adrenergic receptor agonist, a α2-adrenergic receptor agonist, a Mu opioid receptor agonist, a long-acting Mu opioid receptor antagonist, a centrally-acting α adrenergic receptor antagonist combined with a peripherally acting α adrenergic receptor antagonist, a vasoactive/vasopressor agent, a anticholinergic agent, a muscle paralytic, a anticonvulsant, or a membrane-stabilizing agent.

2. The pharmaceutical composition of claim 1(A), further comprising an α1-adrenergic receptor antagonist (A1ARA).

3. The pharmaceutical composition of claim 1, comprising:
(IRNM1) MU+S-A1ARA; or
(IRNM2) MU+A2ARA; or
(IRNM3) MU+NS-A1ARA; or
(IRNM4) MU+S-A1ARA+/−NS-A1ARA; or
(IRNM5) MU+S-A1ARA+/−NS-A1ARA+/−A2ARA; or
(IRNM6) MU+S-A1ARA+/−NS-A1ARA+/−AC or C; or
(IRNM7) MU+S-A1ARA+/−NS-A1ARA+/−AC or C+/−A2ARA; or
(IRMnAW1) MU+S-A1ARA+/−NS-A1ARA; or
(IRMnAW2) MU+S-A1ARA+/−NS-A1ARA+/−VP; or
(IRMnAW3) MU+S-A1ARA+/−NS-A1ARA+/−AC or C; or
(IRMnAW4) MU+S-A1ARA+/−NS-A1ARA+/−VP+AC or C; or
(IRMnAW5) MU+S-A1ARA+/−NS-A1ARA+/−RA; or
(IRMnAW6) MU+S-A1ARA+NS-A1ARA+/−VP+/−RA; or
(IRMnAW7) MU+S-A1ARA+NS-A1ARA+/−VP+RA+AC or C; or
(IRMnAW8) MU+S-A1ARA+NS-A1ARA+VP+RA+AC or C+A2ARA; or
(IRMAW1) MU+S-A1ARA+/−NS-A1ARA; or
(IRMAW2) MU+S-A1ARA+/−NS-A1ARA+/−PMR; or
(IRMAW3) MU+S-A1ARA+NS-A1 ARA+/−VP+/−PMR; or
(IRMAW4) MU+S-A1ARA+NS-A1 ARA+/−PMR+/−AC or C; or
(IRMAW5) MU+S-A1ARA+NS-A1 ARA+/−VP+/−PMR+/−AC or C; or
(Poly1) MU+S-A1 ARA+NS-A1ARA+GCA; or
(Poly2) MU+S-A1 ARA+NS-A1ARA+GCA+AC or C; or
(Poly3) MU+S-A1 ARA+NS-A1ARA+GCA+ASMS; or
(Poly4) MU+S-A1 ARA+NS-A1ARA+GCA+ASMS+AC or C; or
(Poly5) MU+S-A1 ARA+NS-A1ARA+GCA+ASMS+PMR; or
(Poly6) MU+S-A1 ARA+NS-A1ARA+GCA+ASMS+PMR+AC or C; or
(PAOU1) S-A1ARA+VP; or
(PAOU2) S-A1ARA+/−AC or C; or
(PAOU3) S-A1ARA+VP+/−AC or C; or
(PAOU4) NS-A1ARA+VP; or
(PAOU5) NS-A1ARA+AC or C; or
(PAOU6) NS-A1ARA+VP+/−AC or C; or
(PAOU7) S-A1ARA+NS-A1ARA+/−VP+AC or C; or
(PAOU8) S-A1ARA+NS-A1ARA+/−AC or C; or
(PAOU9) S-A1ARA+NS-A1ARA+VP+AC or C; or
(PFR1) MU or MUXR+S-A1ARA+/−NS-A1ARA; or
(PFR2) MU or MUXR+S-A1ARA+NS-A1ARA+/−AC or C; or
(PFR3) MU or MUXR+S-A1ARA+NS-A1 ARA+/−VP; or
(PFR4) MU or MUXR+S-A1ARA+NS-A1ARA+VP+AC or C;
wherein MU=Mu receptor antagonist, XR=timed release, A1ARA=Alpha-1 Adrenergic receptor antagonist, A2ARA=Alpha-2 Adrenergic receptor agonist, VP=Vasopressor, AC=Anticholinergic, C=Cholinergic, PMR=Paralytic/Muscle relaxant, RA=Respiratory Accelerant, GCA=GABA Complex Antagonist, ASMS=Anti-seizure/Membrane stabilizer, PILO=pilocarpine, and wherein each is provided in an amount sufficient to be therapeutically effective.

4. The pharmaceutical composition of claim 1, compromising an α1-adrenergic receptor antagonist that targets α1-adrenergic receptor subtype 1D.

5. The pharmaceutical composition of claim 4, wherein the antagonist that targets α1-adrenergic receptor subtype 1D preferentially targets α1-adrenergic receptor subtype 1D.

6. The pharmaceutical composition of claim 1(A), wherein:
the α2-adrenergic receptor agonist is clonidine; or
the Mu opioid receptor antagonist is naloxone, naltrexone, nalmefene, or a combination of two or more thereof; or
both.

7. The pharmaceutical composition of claim 1, formulated for delivery to a subject.

8. The delivery formulated pharmaceutical composition of claim 7, formulated for intravenous (IV), intramuscular (IM), intranasal (IN), transdermal (TD), intraosseous (10), intrathecal (IT), intraocular (IOC), oral, sublingual (SL), or transtracheal (TT) delivery to the subject.

9. The delivery formulated pharmaceutical composition of claim 7, formulated for injection.

10. The delivery formulated pharmaceutical composition of claim 7, formulated to be delivered to the subject as a premeasured single dose.

11. A kit, comprising:
(A) a container in which is contained the pharmaceutical composition claim 1; or
(B) a first container in which is contained a therapeutically effective amount of a Mu opioid receptor antagonist (MU) and a pharmaceutically acceptable carrier, and second container in which is contained a therapeutically effective amount of a α2-adrenergic receptor agonist (A2ARA) and a pharmaceutically acceptable carrier.

12. The kit of claim 11, comprising one or more containers that collectively contain at least one of the combination:
(IRNM1) MU+S-A1ARA; or
(IRNM2) MU+A2ARA; or
(IRNM3) MU+NS-A1ARA; or
(IRNM4) MU+S-A1ARA+/−NS-A1ARA; or
(IRNM5) MU+S-A1ARA+/−NS-A1ARA+/−A2ARA; or
(IRNM6) MU+S-A1ARA+/−NS-A1ARA+/−AC or C; or
(IRNM7) MU+S-A1ARA+/−NS-A1ARA+/−AC or C+/−A2ARA; or
(IRMnAW1) MU+S-A1ARA+/−NS-A1ARA; or
(IRMnAW2) MU+S-A1ARA+/−NS-A1ARA+/−VP; or
(IRMnAW3) MU+S-A1ARA+/−NS-A1ARA+/−AC or C; or
(IRMnAW4) MU+S-A1ARA+/−NS-A1ARA+/−VP+AC or C; or
(IRMnAW5) MU+S-A1ARA+/−NS-A1ARA+/−RA; or
(IRMnAW6) MU+S-A1ARA+NS-A1ARA+/−VP+/−RA; or
(IRMnAW7) MU+S-A1ARA+NS-A1ARA+/−VP+RA+AC or C; or
(IRMnAW8) MU+S-A1ARA+NS-A1ARA+VP+RA+AC or C+A2ARA; or
(IRMAW1) MU+S-A1ARA+/−NS-A1ARA; or
(IRMAW2) MU+S-A1ARA+/−NS-A1ARA+/−PMR; or
(IRMAW3) MU+S-A1ARA+NS-A1 ARA+/−VP+/−PMR; or
(IRMAW4) MU+S-A1ARA+NS-A1 ARA+/−PMR+/−AC or C; or
(IRMAW5) MU+S-A1ARA+NS-A1 ARA+/−VP+/−PMR+/−AC or C; or
(Poly1) MU+S-A1 ARA+NS-A1ARA+GCA; or
(Poly2) MU+S-A1 ARA+NS-A1ARA+GCA+AC or C; or
(Poly3) MU+S-A1 ARA+NS-A1ARA+GCA+ASMS; or
(Poly4) MU+S-A1 ARA+NS-A1ARA+GCA+ASMS+AC or C; or
(Poly5) MU+S-A1 ARA+NS-A1ARA+GCA+ASMS+PMR; or
(Poly6) MU+S-A1 ARA+NS-A1ARA+GCA+ASMS+PMR+AC or C; or
(PAOU1) S-A1ARA+VP; or
(PAOU2) S-A1ARA+/−AC or C; or
(PAOU3) S-A1ARA+VP+/−AC or C; or
(PAOU4) NS-A1ARA+VP; or
(PAOU5) NS-A1ARA+AC or C; or
(PAOU6) NS-A1ARA+VP+/−AC or C; or
(PAOU7) S-A1ARA+NS-A1ARA+/−VP+AC or C; or
(PAOU8) S-A1ARA+NS-A1ARA+/−AC or C; or
(PAOU9) S-A1ARA+NS-A1ARA+VP+AC or C; or
(PFR1) MU or MUXR+S-A1ARA+/−NS-A1ARA; or
(PFR2) MU or MUXR+S-A1ARA+NS-A1ARA+/−AC or C; or
(PFR3) MU or MUXR+S-A1ARA+NS-A1 ARA+/−VP; or
(PFR4) MU or MUXR+S-A1ARA+NS-A1ARA+VP+AC or C;
wherein MU=Mu receptor antagonist, XR=timed release, A1ARA=Alpha-1 Adrenergic receptor antagonist, A2ARA=Alpha-2 Adrenergic receptor agonist, VP=Vasopressor, AC=Anticholinergic, C=Cholinergic, PMR=Paralytic/Muscle relaxant, RA=Respiratory Accelerant, GCA=GABA Complex Antagonist, ASMS=Anti-seizure/Membrane stabilizer, PILO=pilocarpine, and wherein each is provided in an amount sufficient to be therapeutically effective.

13. A method of preventing or reversing one or more opioid or opiate effects in a subject, comprising: administering to the subject in need of such treatment:
a pharmaceutically acceptable carrier; and one of:
(A) a therapeutically effective amount of a Mu opioid receptor antagonist (MU), and a therapeutically effective amount of a α2-adrenergic receptor agonist (A2ARA); or
(B) a therapeutically effective amount of an α2-adrenergic receptor agonist (A2ARA), a therapeutically effective amount of an α1-adrenergic receptor antagonist (A1ARA), and a therapeutically effective amount of a Mu opioid receptor antagonist (MU); or
(C) a therapeutically effective amount of an α1-adrenergic receptor antagonist, and a therapeutically effective amount of one or more of:
a Mu opioid receptor antagonist (MU), opioid receptor subtype antagonist, opioid receptor subtype agonist, a centrally-acting or peripherally acting respiratory stimulant, a GABA/benzodiazepine receptor complex antagonist, an α1-adrenergic receptor agonist, a α2-adrenergic receptor agonist, a Mu opioid receptor agonist, a long-acting Mu opioid receptor antagonist, a centrally-acting α adrenergic receptor antagonist combined with a peripherally acting α adrenergic receptor antagonist, a vasoactive/vasopressor agent, a anticholinergic agent, a muscle paralytic, a anticonvulsant, or a membrane-stabilizing agent.

14. The method of claim 13, wherein at least one α1 adrenergic receptor antagonist targets α1-adrenergic receptor subtype 1D.

15. The method of claim 14, wherein at least one α1 adrenergic receptor antagonist preferentially targets α1-adrenergic receptor subtype 1D.

16. The method of claim 13(A), wherein:
(i) the α2-adrenergic receptor agonist is clonidine; or
(ii) the Mu opioid receptor antagonist is naloxone, naltrexone, nalmefene, or a combination of two or more thereof; or
(iii) both (i) and (ii).

17. The method of claim 13, wherein the one or more opioid or opiate effects comprise at least one of vocal cord closure (laryngospasm), fentanyl-induced muscle rigidity (FIMR), wooden chest syndrome (WCS), or unconsciousness.

18. The method of claim 13, further comprising identifying the subject as being in need of opiate/opioid or polysubstance overdose reversal before administering the treatment.

19. The method of claim 13, wherein the subject is a human.

20. A method of preventing or reversing one or more opioid or opiate effects in a subject, comprising administering to the subject in need of such treatment the formulated pharmaceutical composition of claim 7.
